(19) United States
(12) Patent Application Publication
(10) Pub. No.: US 2025/0092391 A1
(43) Pub. Date: Mar. 20, 2025

(54) PROMOTERS FOR VIRAL-BASED GENE THERAPY

(71) Applicants: The Regents of the University of California, Oakland, CA (US); The Johns Hopkins University, Baltimore, MD (US)

(72) Inventors: Derek Welsbie, La Jolla, CA (US); Pingwu Zhang, Baltimore, MD (US); Amit Patel, La Jolla, CA (US); Tianlun Lu, La Jolla, CA (US); Donald J. Zack, Baltimore, MD (US); Natalie Chau, La Jolla, CA (US)

(21) Appl. No.: 18/286,984

(22) PCT Filed: Apr. 14, 2022
(86) PCT No.: PCT/US2022/024878

§ 371 Date: Oct. 13, 2023

Related U.S. Application Data
(60) Provisional application No. 63/174,679, filed on Apr. 14, 2021.

(51) Int. Cl. C12N 15/113; A61K 38/00; A61K 48/00; C12N 5/0793; C12N 9/22; C12N 15/86

(52) U.S. Cl. CPC C12N 15/113; A61K 48/005; C12N 5/062; C12N 9/22; C12N 15/86; A61K 38/00; C12N 2310/20; C12N 2510/00; C12N 2750/14143; C12N 2830/205

(57) ABSTRACT

Provided herein, inter alia, are compositions including viruses and nucleic acids having promoters capable of expressing multiple heterologous nucleic acids. The compositions are particularly useful for delivering and expressing heterologous nucleic acids in neurons. With respect to the nervous system, neural tissue, and neurons, the compositions listed herein are contemplated to be effective for treatment of retinal neurodegenerative diseases.

### CROSS-REFERENCES TO RELATED APPLICATIONS

This application claims the benefit of U.S. Provisional Application No. 63/174,679, filed Apr. 14, 2021, which is incorporated herein by reference in entirety and for all purposes.

### STATEMENT AS TO RIGHTS TO INVENTIONS MADE UNDER FEDERALLY SPONSORED RESEARCH AND DEVELOPMENT

This invention was made with government support under 1R01EY029342 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048537-652001WO_SequenceListing_ST25.txt, created Apr. 14, 2022, 12,288 bytes, machine format IBM-PC, MS Windows operating system, is hereby incorporated by reference.

### BACKGROUND

The limited capacity of the amount of genetic information that can be packaged into a virus delivery vehicle is a hurdle in development of gene therapy treatments. Particularly, adeno-associated virus, which is commonly used in gene therapy, has a limited cargo size (packaging capacity) of about 4.7 kilobases, and can deliver a payload of about 4.4 kilobases. Although bidirectional promoters are an attractive solution for expression of multiple genes using a single promoter, bidirectional promoters known in the art do not function well in neurons. Further, although many promoters have some level of bidirectional activity, only a few actually allow for full gene expression in both directions. Reasons for this have not been completely elucidated. In situations in which there are two transgenic promoters, the promoters can often interfere with one another (e.g., Curtin et al., Gene Therapy 15:384-390, 2008; Core et al., Science 322:1845-1848, 2008). For the majority of these promoters, RNA Polymerase II (RNA Pol II) pauses/stops during elongation, thereby resulting in extremely low levels of gene expression.

Thus, there remains a need for bidirectional promoters that can deliver multiple genes. Particularly, bidirectional promoters that function in neurons would enable expression of therapeutic entities for treatment of diseases, for example, neurodegenerative retinal diseases.

Disclosed herein, inter alia, are solutions to these and other problems in the art.

### BRIEF SUMMARY OF THE INVENTION

The present disclosure provides expression constructs including promoters optimized for their compact size and robust expression of two transcripts. The promoters are demonstrated to effectively express genes in neuron cells.

In an aspect is provided a virus having a genome including: i) a promoter having at least 80% sequence identity to the sequence of SEQ ID NO:10 or SEQ ID NO:21; ii) a first heterologous nucleic acid sequence attached to the 3′ end of the promoter; and iii) a second heterologous nucleic acid sequence attached to the 5′ end of the promoter.

In another aspect is provided nucleic acid including: i) a promoter having at least 80% sequence identity to the sequence of SEQ ID NO: 10 or SEQ ID NO:21; ii) a first heterologous nucleic acid sequence attached to the 3′ end of the promoter; and iii) a second heterologous nucleic acid sequence attached to the 5′ end of the promoter.

In another aspect is provided an expression vector including the nucleic acid provided herein including embodiments thereof.

In an aspect is provided a method of expressing a first heterologous nucleic acid sequence and a second heterologous nucleic acid sequence, including contacting a cell with a virus provided herein including embodiments thereof, and allowing the cell to express the first heterologous nucleic acid sequence and the second heterologous nucleic acid sequence.

In an aspect is provided a method of expressing a first heterologous nucleic acid sequence and a second heterologous nucleic acid sequence, including contacting a cell with the nucleic acid provided herein including embodiments thereof or the expression vector provided herein including embodiments thereof, and allowing the cell to express the first heterologous nucleic acid sequence and the second heterologous nucleic acid sequence.

In another aspect is provided a cell including the virus provided herein including embodiments thereof, the nucleic acid provided herein including embodiments thereof, or the expression vector provided herein including embodiments thereof.

In another aspect is provided a method of treating a disease in a subject in need thereof, the method including administering to the subject an effective amount of the virus provided herein including embodiments thereof, the nucleic acid provided herein including embodiments thereof, or the expression vector provided herein including embodiments thereof.

### BRIEF DESCRIPTION OF THE DRAWINGS

FIG. 1. Representative images of HEK 293T cells transfected with construct expressing EGFP and mScarlet with Promoter 10 or control CAG/CMV promoters. Cells were imaged at 10× magnification 24 hrs after transfection.

FIGS. 2A-2B. Fluorescent signal from HEK 293T cells transfected with various promoter constructs. HEK 293T cells were imaged 24 hours after transfection. Cell count and fluorescence intensity are shown. Fluorescent intensities were quantified by an automated algorithm for number of cells and fluorescence intensity (n=4). FIG. 2A: Scarlet expression fluorescent intensities (upper panel) and the number of cells expressing Scarlet (bottom panel).

FIG. 2B: GFP expression fluorescent intensities (upper panel) and the number of cells expressing GFP (bottom panel).

FIG. 3. Representative images of mRGC transfected with Promoter 10 construct (left) and a human H1 promoter reference construct (right). Images were taken 24 hours after transfection at 20× magnification. “EGFP” shows emissions at green wavelengths and “mScarlet” shows emissions at red wavelengths. The “Merge” panel shows that all of the Promoter 10-transfected cells observed emitted light at both wavelengths.

FIGS. 4A-4B. Quantitative data of mRGCs transfected with various candidate bidirectional promoter constructs. Cells were imaged 48 hours after transfection and quantified by an automated algorithm for number of cells and fluorescence intensity (n=4). FIG. 4A: Scarlet expression; FIG. 4B: GFP expression.

FIGS. 5A-5B. Quantitative data showing activity of mouse Promoter 10 and truncated versions of human Promoter 10 compared to human Promoter 10 (SEQ ID NO:10). FIG. 5A: GFP expression; FIG. 5B: m-Scarlet expression.

FIG. 6. Alignment of the human (SEQ ID NO:10) and mouse promoter (SEQ ID NO: 25) nucleotide sequences. A consensus sequence (SEQ ID NO:27) is provided. “N” as used in SEQ ID NO:27 indicates that the nucleotide at that position is not present or is the nucleotide shown in the corresponding position in the mouse or human sequence. Nucleotide designations otherwise correspond to the WIPO standard, ST.26 (2021), Annex 1, Section 1.

FIGS. 7A-7B. Comparison of Cas12 proteins in RFLP gene editing assay. A plasmid expressing Cas 12 protein from one side of human Promoter 10 was combined with a plasmid expressing a single gRNA array containing guide RNAs that target Dlk and Lzk from one side of human Promoter 10. FIG. 7A: Dlk gene editing; FIG. 7B: Lzk gene editing.

FIGS. 8A-8B. Comparison of Dlk and Lzk-gene editing obtained with two-plasmid system (FIG. 7A-7B) to gene editing obtained with a single plasmid to drive expression of Cas12a from one side of human Promoter 10 and a gRNA array from the other side. FIG. 8A: Dlk gene editing; FIG. 8B: Lzk gene editing

FIGS. 9A-9B. Representative images of HEK 293T cells transfected with Promoter 10 (TMEM side)-INTRON-SpyCas9-P2A-EGFP-sNRP terminator. Expression of GFP was detected. Introns assessed include MATLAT1, BRSK2, MAT2A, CCDC115, MDN1, CUEDC2 (FIG. 9A) and SV40, ZDHHC2, SLC35F2, SNF286A, an intron from a gene with an unknown name, MVM No. CBA, and UQCRFS1 (FIG. 9B). Images were taken after 72 hours after transfection at 10× magnification.

FIG. 10. Quantitative data of HEK 293T cells transfected with Promoter 10 (TMEM side)-INTRON-SpyCas9-P2A-EGFP-sNRP terminator. Images were taken after 24 hours of transfection.

FIG. 11. Restriction enzyme disruption assay of Cas12a mediated gene editing of the Dlk locus driven by Promoter 10 (lanes 4-5 and 8-9) or the reverse of Promoter 10 (SEQ ID NO: 26) (lanes 6-7) in transiently transfect N2a cells. Enhancement with an insulator sequence was demonstrated (lanes 8-9).

FIG. 12. Restriction enzyme disruption assay of Cas12a mediated gene editing of the Lzk locus driven by Promoter 10 (lanes 4-5 and 8-9) or the reverse of Promoter 10 (SEQ ID NO: 26) (lanes 6-7) in transiently transfect N2a cells. Enhancement with an insulator sequence was demonstrated (lanes 8-9).

FIG. 13. Representative images of hemagglutinin (HA)-tagged enAsCas12a expression in transiently transfected 293 cells measured by anti-HA immunofluorescent staining. Cpf1 (enAsCas12a) was expressed from either side of promoter 10. LT Cpf1 construct expresses Cpf1 with the TMEM208 side of the promoter. TL Cpf1 construct expresses Cpf1 with the LRRC29 side of the promoter.

FIG. 14. Representative images of mouse retinal ganglion cells (mRGC) transduced with AAV vectors encoding the bidirectional promoter and EGFP and mScarlet. The figure shows a retinal flatmount 14 days after AAV-EGFP-Pro10-mScarlet was intravitreally injected. The image shows robust bidirectional expression of both transgenes. The plane of the image (evidenced by the axons) is in the ganglion cell layer/retinal nerve fiber layer (GCL/RNFL) demonstrating retinal ganglion cell expression of both transgenes.

### DETAILED DESCRIPTION

Provided herein, inter alia, are bidirectional promoters to drive expression of two RNA transcripts in viral vectors used in gene therapy. The bidirectional promoters are contemplated to be particularly useful for delivery of CRISPR components, proteins such as therapeutic proteins, RNAs such as gRNA and inhibitory RNAs. Without limitation, useful bidirectional promoters include promoters comprising SEQ ID NO: 10, truncations thereof, fragments thereof, and variants thereof. In one example, useful bidirectional promoters include promoters comprising SEQ ID NO:21 truncations thereof, fragments thereof, and variants thereof.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non-limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of an adeno-associated viral vector, which may be transduced into a cell. Polynucleotides useful in the methods of the disclosure may comprise natural nucleic acid sequences and variants thereof, artificial nucleic acid sequences, or a combination of such sequences.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford

University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., Cas9) in other proteins with different numbering systems. For example, by performing a simple sequence alignment with a protein (e.g., Cas9) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three-dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
   (see, e.g., Creighton, Proteins (1984)).

Nucleotide sequences are represented according to the WIPO Standard, ST.26 (2021), Annex 1, Section 1 unless otherwise indicated.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. Tis referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The terms “guide RNA”, “gRNA”, “single guide RNA”, and “sgRNA” are used interchangeably and refer to the polynucleotide sequence including the crRNA sequence and optionally the tracrRNA sequence. In embodiments, the gRNA includes the crRNA sequence and the tracrRNA sequence. In embodiments, the gRNA does not include the tracrRNA sequence. The crRNA sequence includes a guide sequence (i.e., “guide” or “spacer”) and a tracr mate sequence (i.e., direct repeat(s)). The term “guide sequence” refers to the sequence that specifies the target site. In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) comprises the tracr mate sequence hybridized to the tracr sequence.

In embodiments, the gRNA is a single-stranded ribonucleic acid. In aspects, the gRNA is from about 10 to about 200 nucleic acid residues in length. In aspects, the gRNA is from about 50 to about 150 nucleic acid residues in length. In aspects, the gRNA is from about 80 to about 140 nucleic acid residues in length. In aspects, the gRNA is from about 90 to about 130 nucleic acid residues in length. In aspects, the gRNA is from about 100 to about 120 nucleic acid residues in length.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system. The C2c1 (“Class 2 candidate 1”) enzyme is a Class II type V-B enzyme. The C2c2 (“Class 2 candidate 2”) enzyme is a Class II type VI-A enzyme. The C2c3 (“Class 2 candidate 3”) enzyme is a Class II type V-C enzyme. Non-limiting exemplary CRISPR associated proteins include Cas1, Cas1B, Cas2, Cas3, Cas4, Cas5, Cas6, Cas7, Cas8, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13

Thus, a “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g., within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase”. In embodiments, Cas9 is an RNA-guided DNA endonuclease enzyme that binds a CRISPR (clustered regularly interspaced short palindromic repeats) nucleic acid sequence.

A “Cfp1” or “Cfp1 protein” as referred to herein includes any of the recombinant or naturally-occurring forms of the Cfp1 (CxxC finger protein 1) endonuclease or variants or homologs thereof that maintain Cfp1 endonuclease enzyme activity (e.g. within at least 50%, 80%, 90%, 95%, 96%, 97%, 98%, 99% or 100% activity compared to Cfp1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cfp1 protein. In embodiments, the Cfp1 protein is substantially identical to the protein identified by the UniProt reference number Q9P0U4 or a variant or homolog having substantial identity thereto.

The term “nuclease-deficient RNA-guided DNA endonuclease enzyme” and the like refer, in the usual and customary sense, to an RNA-guided DNA endonuclease (e.g. a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., single guide RNA (sgRNA)), but is incapable of cleaving the target phosphodiester bond to a significant degree (e.g. there is no measurable cleavage of the phosphodiester bond under physiological conditions). A nuclease-deficient RNA-guided DNA endonuclease thus retains DNA-binding ability (e.g. specific binding to a target sequence) when complexed with a polynucleotide (e.g., sgRNA), but lacks significant endonuclease activity (e.g. any amount of detectable endonuclease activity). In aspects, the nuclease-deficient RNA-guided DNA endonuclease enzyme is a CRISPR-associated protein. In aspects, the nuclease-deficient RNA-guided DNA endonuclease enzyme is dCas9, dCpf1, ddCpf1, Cas-phi, a nuclease-deficient Cas9 variant, a nuclease-deficient Class II CRISPR endonuclease, a zinc finger domain, a transcription activator-like effector (TALE), a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a zinc finger domain, a leucine zipper domain, a winged helix domain, a helix-turn-helix motif, a helix-loop-helix domain, an HMB-box domain, a Wor3 domain, an OB-fold domain, an immunoglobulin domain, or a B3 domain.

In embodiments, the nuclease-deficient RNA-guided DNA endonuclease enzyme is dCas9. The terms “dCas9” or “dCas9 protein” as referred to herein is a Cas9 protein in which both catalytic sites for endonuclease activity are defective or lack activity. In aspects, the dCas9 protein has mutations at positions corresponding to D10A and H840A of S. pyogenes Cas9. In aspects, the dCas9 protein lacks endonuclease activity due to point mutations at both endonuclease catalytic sites (RuvC and HNH) of wild type Cas9. The point mutations can be D10A and H840A. In aspects, the dCas9 has substantially no detectable endonuclease (e.g., endodeoxyribonuclease) activity.

In embodiments, the nuclease-deficient RNA-guided DNA endonuclease enzyme is “ddCpf1” or “ddCas12a”. The terms “DNAse-dead Cpf1” or “ddCpf1” refer to mutated Acidaminococcus sp. Cpf1 (AsCpf1) resulting in the inactivation of Cpf1 DNAse activity. In aspects, ddCpf1 includes an E993A mutation in the RuvC domain of AsCpf1. In aspects, the ddCpf1 has substantially no detectable endonuclease (e.g., endodeoxyribonuclease) activity.

The term “RNA-guided RNA nuclease” or “RNA-guided RNase” and the like refer, in the usual and customary sense, to an RNA-guided nuclease that targets a specific phosphodiester bond within an RNA polynucleotide, wherein the recognition of the phosphodiester bond is facilitated by a separate polynucleotide sequence (for example, a RNA sequence (e.g., single guide RNA (sgRNA), a guide RNA (gRNA)). Typically, an RNA guided RNase targets single-stranded RNA. In aspects, the RNA-guided RNase is Cas13 (e.g. Cas13a, Cas13b).

A “Cas13a” or “Cas13a protein” as referred to herein includes any of the recombinant or naturally-occurring forms of the Cas13a (CRISPR-associated endoribonuclease Cas13a) endonuclease, also known as CRISPR-associated endoribonuclease C2c2, C2c2, or variants or homologs thereof that maintain Cas13a endonuclease enzyme activity (e.g. within at least 50%, 80%, 90%, 95%, 96%, 97%, 98%, 99% or 100% activity compared to Cas13a). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13a protein. In embodiments, the Cas13a protein is substantially identical to the protein identified by the UniProt reference number C7NBY4 or a variant or homolog having substantial identity thereto.

A “Cas13b” or “Cas13b protein” as referred to herein includes any of the recombinant or naturally-occurring forms of the Cas13b (CRISPR-associated RNA-guided ribonuclease Cas13b) endonuclease, or variants or homologs thereof that maintain Cas13b nuclease enzyme activity (e.g. within at least 50%, 80%, 90%, 95%, 96%, 97%, 98%, 99% or 100% activity compared to Cas13b). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13b protein. In embodiments, the Cas13b protein is substantially identical to the protein identified by the UniProt reference number A0A8G0P913 or a variant or homolog having substantial identity thereto.

The term “Krüppel associated box domain” or “KRAB domain” as provided herein refers to a category of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, 2017; Lambert et al. The human transcription factors, Cell 172, 2018; Gilbert et al., Cell (2013); and Gilbert et al., Cell (2014). In aspects, the KRAB domain is a KRAB domain of Kox 1.

The term “VP64” or “VP64 protein” as provided herein includes any of the recombinant or naturally-occurring forms of Tegument protein VP16 (VP64), also known as Alpha trans-inducing protein, Alpha-TIF, or variants or homologs thereof that maintain VP64 protein activity (e.g. within at least 50%, 80%, 90%, 95%, 96%, 97%, 98%, 99% or 100% activity compared to VP64 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VP64 protein polypeptide. In embodiments, VP64 protein is the protein as identified by the UniProt reference number P06492, or a variant, homolog or functional fragment thereof.

The term “Rta” or “Rta protein” as provided herein includes any of the recombinant or naturally-occurring forms of Replication and transcription activator (Rta), also known as R transactivator, Immediate-early protein Rta, or variants or homologs thereof that maintain Rta protein activity (e.g. within at least 50%, 80%, 90%, 95%, 96%, 97%, 98%, 99% or 100% activity compared to Rta protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Rta protein polypeptide. In embodiments, Rta protein is the protein as identified by the UniProt reference number P03209, or a variant, homolog or functional fragment thereof.

The term “p65” or “p65 protein” as provided herein includes any of the recombinant or naturally-occurring forms of Transcription factor p65 (p65), also known as Nuclear factor NF-kappa-B p65 subunit, or variants or homologs thereof that maintain p65 protein activity (e.g. within at least 50%, 80%, 90%, 95%, 96%, 97%, 98%, 99% or 100% activity compared to p65 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring p65 protein polypeptide. In embodiments, p65 protein is the protein as identified by the UniProt reference number Q04206, or a variant, homolog or functional fragment thereof.

The term “DNA methyltransferase” as provided herein refers to an enzyme that catalyzes the transfer of a methyl group to DNA. Non-limiting examples of DNA methyltransferases include Dnmt1, Dnmt3A, and Dnmt3B. In aspects, the DNA methyltransferase is mammalian DNA methyltransferase. In aspects, the DNA methyltransferase is human DNA methyltransferase. In aspects, the DNA methyltransferase is mouse DNA methyltransferase. In aspects, the DNA methyltransferase is a bacterial cytosine methyltransferase and/or a bacterial non-cytosine methyltransferase. Depending on the specific DNA methyltransferase, different regions of DNA are methylated. For example, Dnmt3A typically targets CpG dinucleotides for methylation. Through DNA methylation, DNA methyltransferases can modify the activity of a DNA segment (e.g., gene expression) without altering the DNA sequence. In aspects, DNA methylation results in repression of gene transcription and/or modulation of methylation sensitive transcription factors or CTCF. As described herein, fusion proteins may include one or more (e.g., two) DNA metyltransferases. When a DNA methyltransferase is included as part of a fusion protein, the DNA methyltransferase may be referred to as a “DNA methyltransferase domain.”

A “Dnmt3A”, “Dnmt3a,” “DNA (cytosine-5)-methyltransferase 3A” or “DNA methyltransferase 3a” protein as referred to herein includes any of the recombinant or naturally-occurring forms of the Dnmt3A enzyme or variants or homologs thereof that maintain Dnmt3A enzyme activity (e.g. within at least 50%, 55%, 60%, 65%, 70%, 75%, 80%, 85%, 90%, 91%, 92%, 93%, 94%, 95%, 96%, 97%, 98%, 99% or 100% activity compared to Dnmt3A). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Dnmt3A protein. In aspects, the Dnmt3A protein is substantially identical to the protein identified by the UniProt reference number Q9Y6K1 or a variant or homolog having substantial identity thereto.

A “dual leucine zipper kinase”, “DLK” or “DLK protein” is a mitogen-activated protein kinase kinase kinase (MAP3K) mixed lineage kinase family member and member of the ser/thr kinase superfamily. DLK plays a role in neural kinase signaling pathways, including neuronal cell death signaling. DLK comprises an N-terminal domain, a catalytic domain, a leucine zipper domain comprising two leucine zippers, and a C-terminal domain. Human DLK is encoded by the mitogen-activated protein kinase kinase kinase 12 gene (MAP3K12), which is cytogenetically localized to chromosome region 12q13.13. A “human DLK” refers to any allelic form encoded by a human MAP3K12 gene, including splice variants. In aspects, the DLK protein is substantially identical to the proteins identified in UniProt reference number Q12852, e.g., isoform 1 and isoform 2 as designated in UniProtKB Q12852. “DLK” may refer to human and non-human DLK polypeptides and polynucleotides, e.g., mammalian DLK sequences, such as mouse or rat DLK sequences.

“Leucine zipper kinase”, “LZK”, or “LZK protein” is an MAP3K family member structurally related to DLK that also plays a role in neural signaling pathways, including neuronal cell death. LZK comprises an N-terminal domain, a catalytic domain (“kinase domain”), a leucine zipper domain comprising two leucine zippers, and a C-terminal domain. Human LZK is encoded by the mitogen-activated protein kinase kinase kinase 13 gene (MAP3K13), which is cytogenetically localized to chromosome region 3q27.2. A “human LZK” refers to any allelic form encoded by a human MAP3K13 gene, including splice variants. Illustrative human LZK polypeptide sequences are available under UniProtKB entry 043283. Isoform 1 (O42283-1) is designated in the UniProt entry as the canonical isoform. “LZK” may refer to human and non-human LZK polypeptides and polynucleotides, e.g., mammalian LZK sequences, such as mouse and rat DLK sequences. In aspects, the LZK protein is substantially identical to the proteins identified in the UniProt reference number 043283.

The term “dominant negative” with respect to DLK or LZK refers to a dominant negative DLK protein variant (dnDLK) or dominant negative form of LZK (dnLZK) e.g., a leucine zipper domain of LZK or variant thereof that is neuroprotective when expressed in neurons in which endogenous wildtype DLK is expressed. Without intending to be bound by a particular mechanism, dnDLK may inhibit DLK homodimerization, and dominant negative LZK may inhibit DLK-LZK heterodimerization.

“SARM1” or “SARM1 protein” as referred to herein includes any of the recombinant or naturally-occurring forms of the SARM1, also known as NAD(+) hydrolase SARM1, or NADase SARM1, or variants or homologs thereof that maintain SARM1 activity (e.g. within at least 50%, 55%, 60%, 65%, 70%, 75%, 80%, 85%, 90%, 91%, 92%, 93%, 94%, 95%, 96%, 97%, 98%, 99% or 100% activity compared to SARM1). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring SARM1 protein. In aspects, the SARM1 protein is substantially identical to the protein identified by the UniProt reference number Q6SZW1 or a variant or homolog having substantial identity thereto. The term “dominant negative” in relation to SARM1 refers to a dominant negative SARM1 (dnSARM1) or variant thereof that is neuroprotective. Without wishing to be bound by scientific theory, dominant negative SARM1 may form non-functional complexes with wildtype SARM1, thereby inhibiting or downregulating axon degenerating activity of wildtype SARM1.

“NMNAT1” or “NMNAT1 protein” as referred to herein includes any of the recombinant or naturally-occurring forms of the NMNAT1, also known as Nicotinamide/nicotinic acid mononucleotide adenylyltransferase 1, Nicotinamide-nucleotide adenylyltransferase 1, or variants or homologs thereof that maintain NMNAT1 activity (e.g. within at least 50%, 55%, 60%, 65%, 70%, 75%, 80%, 85%, 90%, 91%, 92%, 93%, 94%, 95%, 96%, 97%, 98%, 99% or 100% activity compared to NMNAT1). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring NMNAT1 protein. In aspects, the NMNAT1 protein is substantially identical to the protein identified by the UniProt reference number Q9HAN9 or a variant or homolog having substantial identity thereto. Cytoplasmic NMNAT is one of the three human NMNAT paralogs. Cytoplasmic NMNAT may be enriched in the brain and is capable of inhibiting neuronal cell death (Tang, B. L. Why is NMNAT Protective against Neuronal Cell Death and Axon Degeneration, but Inhibitory of Axon Regeneration? Cells 2019, 8, 267. https://doi.org/10.3390/cells8030267).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

Single chain antibodies such as nanobodies may be expressed using the bispecific promoter. Nanobodies are well known and described in, for example, Morrison C., Nat Rev Drug Dis 18:485-487, 2019; Pedersen D. V., et al., Mol Immunol 124:200-210, 2020.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A nucleic acid is “operably linked” when it is placed into a functional relationship with another nucleic acid sequence (e.g. a promoter). Broadly speaking, a promoter (e.g. promoter10, etc.) or enhancer is operably linked to a coding sequence (e.g. a first heterologous nucleic acid sequence, a second heterologous nucleic acid sequence) if it affects the transcription of the sequence. In general, a ribosome binding site is operably linked to a coding sequence if it is positioned so as to facilitate translation. Operably linked means that the nucleotide sequences being linked are typically contiguous. However, as enhancers generally function when separated from the promoter by several kilobases and intronic sequences may be of variable lengths, some polynucleotide elements may be operably linked but not directly flanked and may even function in trans from a different allele or chromosome. Linking may be accomplished by ligation at convenient sites. If such sites do not exist, the synthetic oligonucleotide adaptors or linkers are used in accordance with conventional practice. In relation to promoters described herein, the promoter is operably linked (or, equivalently “operably positioned”) to coding sequences (e.g. the first heterologous nucleic acid sequence and the second heterologous nucleic acid sequence) 5′ to the promoter and/or 3′ to the promoter when the promoter is in a location and/or orientation in relation to the coding sequence(s) to control (increase) transcription of the sequence(s). The coding sequence(s) (e.g. the first heterologous nucleic acid sequence and the second heterologous nucleic acid sequence) can be, for illustration and not limitation, independently selected from DNA, mRNA, guide RNA, and inhibitory RNA (e.g., snRNA, miRNA, siRNA, shRNA).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “variant” or “derivative” in the context of polynucleotide (e.g. nucleic acid sequence or oligonucleotide) or peptide (e.g. an amino acid sequence or protein) may refer to a polynucleotide sequence or peptide sequence that has some sequence similarity to their reference sequence. In some examples, a variant or derivative can have at least 30%, 35%, 40%, 45%, 50%, 55%, 60%, 65%, 70%, 75%, 80%, 85%, 90%, 95%, 96%, 97%, 98% or 99% sequence identity (or equivalently used with similarity or homology) to its reference sequence. The terms “functional derivative” or “functional variant” in the context of polynucleotide or peptide sequence may refer to any variant or derivative that maintains the activity to a substantial level, e.g. at least 30% or more of the activity of the reference sequence.

The term “recombinant” when used with reference, e.g., to a virus, cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, a recombinant virus is generated by combining portions of nucleic acids using recombinant nucleic acid technology. For example, a recombinant virus may be generated by replacing one or more viral genes with a heterologous gene. For example, a recombinant virus may be generated by replacing a viral promoter with a heterologous promoter (e.g. a heterologous promoter (e.g. promoter 10, etc.)). Thus, in embodiments, the virus provided herein including embodiments thereof is a recombinant virus. In instances, recombinant cells express genes that are not found within the native (non-recombinant) form of the cell or express native genes that are otherwise abnormally expressed, under expressed or not expressed at all. Thus, in embodiments, the cell (e.g. neuron, RGC, photoreceptor cell, RPE cell, etc.) provided herein including embodiments thereof is a recombinant cell. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein). When used in reference to a nucleic acid sequence, “heterologous” indicates that the nucleic acid sequence is not found in the same relationship in nature to one or more separate nucleic acid sequences (e.g. a promoter (e.g. promoter10)), wherein the nucleic acid sequence and the one or more separate nucleic acid sequences are subsequences within a longer nucleic acid sequence. For instance, the longer sequence is typically recombinantly produced, having two or more subsequences from unrelated genes arranged to make a new functional nucleic acid, e.g., a promoter from one source and a coding region from another source. Similarly, a heterologous peptide sequence indicates that the peptide sequence is not found in the same relationship in nature to a separate peptide sequence, wherein the peptide sequence and the separate peptide sequence are subsequences within a longer peptide (e.g. a fusion protein). A nucleic acid or polypeptide is also considered to be “heterologous” to a cell if it a modified version of a nucleic acid or polypeptide that naturally occurs in the cell, or if a genetic manipulation to the cell results in expression that is altered, e.g., overexpressed, relative to the level expression in the cell without the genetic modification.

The term “exogenous” refers to a molecule or substance (e.g., a compound, nucleic acid or protein) that originates from outside a given cell, organism, or virus. Conversely, the term “endogenous” refers to a molecule or substance that is native to, or originates within, a given cell, organism, or virus. In embodiments, the promoter (e.g. Promoter 10) provided herein including embodiments thereof is exogenous to the virus (e.g. AAV, etc.) provided herein including embodiments thereof. In embodiments, the first heterologous nucleic acid sequence and the second heterologous nucleic acid sequence is exogenous to the virus (e.g. AAV, etc.) provided herein, including embodiments thereof.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A nucleic acid that is the predominant species present in a preparation is substantially purified.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector (e.g. adeno-associated viral vector) may be used in the methods described herein. Examples for viral vectors include, but are not limited to retroviral, adenoviral, lentiviral and adeno-associated viral (AAV) vectors. In embodiments, the viral vector is an adenoviral vector. In embodiments, the viral vector is an AAV vector. In some embodiments, the nucleic acid molecules are introduced into a cell using an AAV vector following standard procedures well known in the art. The terms “transfection” or “transduction” also refer to introducing proteins into a cell from the external environment. In embodiments, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Transduce” or “transduction” are used according to their plain ordinary meanings and refer to the process by which one or more foreign nucleic acids (i.e. DNA not naturally found in the cell) are introduced into a cell. Typically, transduction occurs by introduction of a virus or viral vector (e.g. AAV vector) into the cell. For example, an AAV vector including a bidirectional promoter may be transduced into a cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, an virus as described herein and a neuron. In embodiments contacting includes, for example, allowing a virus as described herein to physically touch a cell (e.g. a neuron). In embodiments contacting includes, for example, allowing a nucleic acid as described herein to physically touch a cell (e.g. a neuron).

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., Spodoptera) and human cells. In embodiments, the cell is a human cell. In embodiments, the cell is a ganglion cell. In embodiments, the cell is a neuronal cell. Cells may be useful when they are naturally nonadherent or have been treated not to adhere to surfaces, for example by trypsinization.

The term “retinal ganglion cell” or “RGC” refers to a type of neuron located near an inner surface (the ganglion cell layer) of the retina of the eye. RGCs receive information from photoreceptors via two intermediate neuron types: bipolar cells and amacrine cells and transmit information to regions of the brain. RGCs vary greatly in terms of size and responses to visual stimulation but all have long axons that extend to the brain. RGC loss is a hallmark of numerous optic neuropathies, including glaucoma.

The term “photoreceptor cell” refers to a type of neuroepithelial cell found in the retinal. Photoreceptor cells absorb photons, which trigger a change in the cell membrane potential to stimulate a sensory response. Rod photoreceptor cells are sensitive to light and operate under dim lighting conditions. Cone photoreceptor cells function under ambient and bright lighting conditions, exhibit rapid responses to variations in light intensity, and are responsible for color vision and high visual acuity. Poor photoreceptor development, function or survival are characteristic of retinal diseases including retinitis pigmentosa and macular degeneration.

The term “retinal pigment epithelium cell” or “RPE cell”, refers to the cells that form the pigmented layer outside the retina. The pigmented layer is located between the Bruch's membrane (choroid inner border) and the photoreceptors. The RPE is an intermediate for supplying nutrients to the retina, and assists in numerous functions, including retina development, absorption of light, secretion of growth factors, and mediating the immune response of the eye. Dysfunction of the RPE may lead to vision loss or blindness in conditions including retinitis pigmentosa, diabetic retinopathy, West Nile virus, and macular degeneration.

The terms “virus” or “virus particle” refers to a virion having a viral genome (e.g. DNA, RNA, single-stranded, double-stranded). In embodiments, the virus includes a viral capsid and associated proteins, and in some cases (e.g. herpesvirus, poxvirus), an envelope including lipids and optionally components of host cell membranes, and/or viral proteins.

“Poxvirus” refers to a member of Poxviridae family capable of infecting vertebrates and invertebrates which replicate in the cytoplasm of their host. In embodiments, poxvirus virions have a size of about 200 nm in diameter and about 300 nm in length and possess a genome in a single, linear, double-stranded segment of DNA, typically 130-375 kilobase. In embodiments, the poxvirus is a vaccinia virus.

The term “herpes simplex virus” or “HSV” refers to members of the Herpesviridae family. Herpes simplex virus 1 and 2 (HSV-1 and HSV-2) are two members of the human Herpesviridae family, a set of viruses that produce viral infections in the majority of humans. The Herpesviridae are a large family of DNA viruses that all share a common structure and are composed of relatively large double-stranded, linear DNA genomes encoding 100-200 genes encapsided within an icosahedral capsid which is enveloped in a lipid bilayer membrane. The oncolytic herpes virus can be derived from different types of HSV. In aspects, the oncolytic herpes virus is HSV-1. In aspects, the oncolytic herpes virus is HSV-2.

Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera.

As used herein, the term “lentivirus” refers to a group (or genus) of complex retroviruses. Lentiviruses have RNA genomes and include the reverse transcriptase enzyme that converts RNA into DNA before integration into the host cells genome. Lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

The term “replicate” is used in accordance with its plain ordinary meaning and refers to the ability of a cell or virus to produce progeny. A person of ordinary skill in the art will immediately understand that the term replicate, when used in connection with DNA, refers to the biological process of producing two identical replicas of DNA from one original DNA molecule. Thus, the term “replicate” includes passaging and re-infecting progeny cells. In the context of a virus, the term “replicate” includes the ability of a virus to replicate (duplicate the viral genome and packaging said genome into viral particles) in a host cell and subsequently release progeny viruses from the host cell, which results in the lysis of the host cell.

The term “plaque forming units” is used according to its plain ordinary meaning in Virology and refers to the amount of plaques in a cell monolayer that can be formed per volume of viral particles. In some embodiments the units are based on the number of plaques that could form when infecting a monolayer of susceptible cells. For example, in embodiments 1,000 PFU/μl indicates that 1 μl of a solution including viral particles contains enough virus particles to produce 1000 infectious plaques in a cell monolayer. In embodiments, plaque forming units are abbreviated “PFU”.

The terms “multiplicity of infection” or “MOI” are used according to its plain ordinary meaning in Virology and refers to the ratio of infectious agent (e.g., adeno-associated virus) to the target (e.g., cell) in a given area or volume. In embodiments, the area or volume is assumed to be homogenous.

The terms “disease” or “condition” refer to a state of being or health status of a patient or subject capable of being treated with the compounds or methods provided herein. The disease may be a neurodegenerative disease. In embodiments, the neurodegenerative disease is glaucoma (including open-angle and narrow/closed-angle glaucoma, primary and secondary glaucoma, normal tension and high-IOP glaucoma), age-related macular degeneration (AMD) including dry (non-exudative) and wet (exudative, neovascular) AMD, choroidal neovascularization (CNV), choroidal neovascular membranes (CNVM), cystoid macular oedema (CME), epiretinal membranes (ERM) and macular perforations, myopia-associated choroidal neovascularization, angioid and vascular streaks, retinal detachment, diabetic retinopathy, diabetic macular oedema (DME), atrophic and hypertrophic lesions in the retinal pigment epithelium, retinal vein occlusion, choroidal retinal vein occlusion, macular oedema, macular oedema associated with renal vein occlusion, retinitis pigmentosa and other inherited retinal degenerations (e.g. Stargardt disease), retinopathy of prematurity, other optic neuropathies including toxic optic neuropathy (e.g. methanol, ethambutol), nonarteritic ischemic optic neuropathy, arteritic ischemic optic neuropathy/giant cell arteritis, traumatic optic neuropathy (including traumatic brain injury), idiopathic intracranial hypertension/pseudotumor cerebri, inflammatory optic neuropathies (e.g. optic neuritis), compressive optic neuropathies (e.g. pituitary adenoma), infiltrative optic neuropathies (e.g. sarcoidosis, lymphoma), autoimmune optic neuropathies, lipid storage diseases (e.g. Tay-Sachs), nutritional optic neuropathies, Leber's hereditary optic neuropathy, dominant optic atrophy, Friedrich's ataxia, radiation-induced optic neuropathy, iatrogenic optic neuropathies, space flight-associated neuro-ocular syndrome (SANS), inflammation disorders of the eye, for example uveitis, scleritis, cataract, refraction anomalies, for example myopia, hyperopia, astigmatism or keratoconus, neurotrophic keratopathy, corneal denneratvation and promoting corneal reinnervation and diabetic keratopathy.

In embodiments, the disease is a neurodegenerative non-ophthalmological disorder. Neurodegenerative non-ophthalmological disorders which may be treated using the compositions (e.g. viruses and nucleic acids) provided herein include, but not limited to: Amyotrophic lateral sclerosis (ALS), Alzheimer's disease, Parkinson's disease, traumatic brain injury, Parkinson's-plus disease, Huntington's disease, peripheral neuropathies, ischemia, stroke, intracranial haemorrhage, cerebral haemorrhage, nerve damage caused by exposure to toxic compounds selected from the group consisting of heavy metals, industrial solvents, drugs and chemotherapeutic agents, injury to the nervous system caused by physical, mechanical or chemical trauma trigeminal neuralgia, glossopharyngeal neuralgia, Bell's Palsy, myasthenia gravis, muscular dystrophy, progressive muscular atrophy, primary lateral sclerosis (PLS), spinal muscular atrophy, inherited muscular atrophy, invertebrate disk syndromes, cervical spondylosis, plexus disorders, thoracic outlet destruction syndromes, porphyria, pseudobulbar palsy, progressive bulbar palsy, multiple system atrophy, progressive supranuclear palsy, corticobasal degeneration, dementia with Lewy bodies, frontotemporal dementia, demyelinating diseases, Guillain-Barre syndrome, multiple sclerosis, Charcot-Marie-Tooth disease, prion disease, Creutzfeldt-Jakob disease, Gerstmann-Sträussler-Scheinker syndrome (GSS), fatal familial insomnia (FFI), bovine spongiform encephalopathy, Pick's disease, epilepsy, AIDS demential complex. In some embodiments, a dominant negative polypeptide as described herein is employed to prevent herpesvirus reactivation. In some embodiments, a dominant negative polypeptide as described herein is employed to prevent aberrant neural cell regeneration. In some embodiments, the disorder is chemotherapy-induced peripheral neuropathy (CIPN), e.g. nerve damage caused by exposure to chemotherapeutic agents. In some embodiments, the disease is a hearing loss disorder, for example, age-related hearing loss, chemotherapy-induced hearing loss, hereditary hearing loss, aminoglycoside-induced hearing loss, trauma-induced hearing loss, and noise-induced hearing loss

The term “associated” or “associated with” in the context of a substance or substance activity or function associated with a disease (e.g. glaucoma) is caused by (in whole or in part), or a symptom of the disease is caused by (in whole or in part) the substance or substance activity or function.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

“Patient” or “subject in need thereof” refers to a living organism suffering from or prone to a disease (e.g. glaucoma, etc.) or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

For prophylactic use, a therapeutically effective amount of the composition described herein are administered to a subject prior to or during early onset (e.g., upon initial signs and symptoms of cancer). Therapeutic treatment involves administering to a subject a therapeutically effective amount of the agents described herein after diagnosis or development of disease.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. In some embodiments, promoter 10 expression constructs and compositions as described herein are administered to neuronal cells, such as sensory neurons. In some embodiments, promoter 10 expression compositions are administered to retinal cells, e.g., RGCs, photoreceptor cells, RPE cells, or ophthalmic neurons, such as bipolar cells, amacrine cells or horizontal cells. In some embodiments, administration is by intraocular delivery, e.g., intravitreal injection.

By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The pharmaceutical preparation is optionally in unit dosage form. In such form the preparation is subdivided into unit doses containing appropriate quantities of the active component.

### Viral Compositions

Provided herein, inter alia, are viral compositions useful for expressing nucleic acid sequences (e.g. a first heterologous nucleic acid sequence, a second heterologous nucleic acid sequence) in a cell. Viral expression systems can be limited by the capacity of nucleotides which may be packaged into a viral genome (e.g. a viral vector). For example, adenoviruses (Ad) have a capacity of about 8.5 kilobases, adeno-associated viruses (AAV) have a capacity of about 4.7 kilobases, and lentiviruses have a capacity of about 8.5 kilobases. Applicant has identified promoters that allow expression of two or more heterologous nucleic acid sequences in a virus despite limitations in the capacity of the viral genome. Therefore, the need for a second promoter is avoided. For example, the promoter provided herein allows production of two separate transcripts in opposite directions (e.g. in a sense orientation from one strand of a double-stranded DNA, and in an antisense orientation from the second strand of the double-stranded DNA). The promoter therefore allows simultaneous expression of two operably linked nucleic acid sequences.

A “promoter” as used herein is a transcription regulatory sequence which is capable of directing transcription of a nucleic acid segment (e.g., a transgene having, for example, an open reading frame) when operably connected thereto. In embodiments, the promoter includes a sequence having at least 80% sequence identity to SEQ ID NO: 10, 21, 25 or 26. In embodiments, the promoter has at least 80%, 85%, 90%, 95%, 96%, 97%, 98% or 99% sequence identity to the sequence of SEQ ID NO: 10. In embodiments, the promoter has at least 80%, 85%, 90%, 95%, 96%, 97%, 98% or 99% sequence identity to the sequence of SEQ ID NO:21. In embodiments, the promoter has at least 80%, 85%, 90%, 95%, 96%, 97%, 98% or 99% sequence identity to the sequence of SEQ ID NO:25. In embodiments, the promoter has at least 80%, 85%, 90%, 95%, 96%, 97%, 98% or 99% sequence identity to the sequence of SEQ ID NO:26.

The promoter provided herein is capable of functioning as a bidirectional promoter. A “bidirectional promoter” as used herein is a transcriptional regulatory sequence located in an intergenic region between two genes that are located on complementary strands of DNA. The bidirectional promoter directs transcription of the two genes in opposite directions. For example, a bidirectional promoter is capable of conferring transcription of the two genes in sense and antisense orientations. Thus, in embodiments, the promoter is a bidirectional promoter.

As used herein, “heterologous nucleic acid sequence” refers to a nucleic acid sequence that is not operably linked in nature to the promoter provided herein including embodiments thereof. Thus, the heterolous nucleic acid sequence provided herein is not natively found in the same relationship to the promoter (e.g. promoter10, etc.) provided herein. Heterologous nucleic acid sequences provided herein include sequences encoding therapeutic or detectable agents. The viruses provided herein may be useful when it is not desirable or feasible to use viral 2A elements or weak IRES elements in a viral genome (e.g. viral vector). Thus, in embodiments, the virus provided herein does not include a viral 2A element or an IRES element. In embodiments, the virus provided herein does not include a viral 2A element. In embodiments, the virus does not include an IRES element.

In an aspect is provided a virus having a genome including: i) a promoter having at least 80% sequence identity to the sequence of SEQ ID NO: 10 or SEQ ID NO:21; ii) a first heterologous nucleic acid sequence attached to the 3′ end of the promoter; and iii) a second heterologous nucleic acid sequence attached to the 5′ end of the promoter. In embodiments, the promoter is exogenous to the virus. For example, the promoter provided herein does not originate from the virus provided herein including embodiments thereof.

“Attach” or “attachment” refers to direct or indirect covalent attachment of a nucleic acid sequence to a separate nucleic acid sequence. For example, a nucleic acid sequence (e.g. promoter) may be directly attached to a separate nucleic acid sequence (e.g. a first or a second heterologous nucleic acid sequence) with no intervening sequence in between. Alternatively, a nucleic acid sequence (e.g. promoter) may be indirectly attached to a separate nucleic acid sequence (e.g. a first or a second heterologous nucleic acid sequence) though one or more intervening nucleic acid sequences (e.g. a linker sequence, an intron, an insulator sequence, etc.). Thus, attachment of a first heterologous nucleic acid sequence to the 3′ end of the promoter can be direct attachment with no intervening sequence in between. Alternatively, attachment of a first heterologous nucleic acid sequence to the 3′ end of the promoter can be indirect attachment with one or more intervening sequences (e.g. an intron) linking the first heterologous nucleic acid sequence to the promoter.

The promoter provided herein allows for expression of a first therapeutic or detectable agent encoded by a first heterologous nucleic acid sequence, and expression of a second therapeutic or detectable agent encoded by the reverse complement of the second heterologous nucleic acid. For example, replication of a single-stranded (ss) genome allows for expression of the first therapeutic agent or detectable agent from one strand, and expression of the second therapeutic or detectable agent on the complementary strand. In another example, a double-stranded (ds) genome allows for expression of the first therapeutic agent or detectable agent from one strand, and expression of the second therapeutic or detectable agent on the complementary strand.

A “therapeutic agent” as used herein refers to an agent (e.g., compound or composition described herein) that when administered to a subject will have the intended prophylactic effect or the intended therapeutic effect. The intended prophylactic effect can include preventing or delaying the onset (or reoccurrence) of an injury, disease, pathology or condition, or reducing the likelihood of the onset (or reoccurrence) of an injury, disease, pathology, or condition, or their symptoms). The intended therapeutic effect can included treatment or amelioration of an injury, disease, pathology or condition, or their symptoms, including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being. The intended prophylactic or therapeutic effect can be achieved by modulating the activity or function of a molecule (e.g. a gene, a protein) associated with a disease or symptoms of the disease. For instance, a therapeutic agent may modify a variant of a gene associated with the disease. A therapeutic agent may decrease the activity of a protein associated with the disease. In another example, the therapeutic agent may modify the levels of a molecule associated with a disease or symptoms of the disease. For instance, a therapeutic agent may inhibit expression of a gene associated with the disease. A product (e.g., the first or second therapeutic agent) “encoded by a nucleic acid” may be a polypeptide (e.g. Cas9, etc.) or nucleic acid (e.g. gRNA, etc.).

A “detectable agent” as used herein refers to a composition detectable by spectroscopic, photochemical, biochemical, immunochemical, chemical, or other physical means. The detectable agent may be a protein. For example, a detectable agent may be a fluorescent or luminescent protein. Fluorescent proteins include mCherry, Emerald, green fluorescent protein (GFP), and enhanced GFP (EGFP). Fluorescent proteins include red, blue, yellow, cyan, and sapphire fluorescent proteins, and reef coral fluorescent protein. Luminescent proteins include luciferase, aequorin and derivatives thereof.

In embodiments, the first heterologous nucleic acid sequence encodes a first therapeutic agent or a first detectable agent and the second heterologous nucleic acid sequence is the reverse complement of a sequence encoding a second therapeutic agent or a second detectable agent, or the first heterologous nucleic acid sequence encodes the second therapeutic agent or the second detectable agent and the second heterologous nucleic acid sequence is the reverse complement of a sequence encoding the first therapeutic agent or the first detectable agent. In embodiments, the first heterologous nucleic acid sequence encodes a first therapeutic agent or a first detectable agent and the second heterologous nucleic acid sequence is the reverse complement of a sequence encoding a second therapeutic agent or a second detectable agent. In embodiments, the first heterologous nucleic acid sequence encodes the second therapeutic agent or the second detectable agent and the second heterologous nucleic acid sequence is the reverse complement of a sequence encoding the first therapeutic agent or the first detectable agent.

In embodiments, the first heterologous nucleic acid sequence encodes a first therapeutic agent and the second heterologous nucleic acid sequence is the reverse complement of a sequence encoding a second therapeutic agent, or the first heterologous nucleic acid sequence encodes the second therapeutic agent and the second heterologous nucleic acid sequence is the reverse complement of a sequence encoding the first therapeutic agent. In embodiments, the first heterologous nucleic acid sequence encodes a first therapeutic agent and the second heterologous nucleic acid sequence is the reverse complement of a sequence encoding a second therapeutic agent. In embodiments, the first heterologous nucleic acid sequence encodes the second therapeutic agent and the second heterologous nucleic acid sequence is the reverse complement of a sequence encoding the first therapeutic agent.

In embodiments, the first heterologous nucleic acid sequence encodes a first detectable agent and the second heterologous nucleic acid sequence is the reverse complement of a sequence encoding a second detectable agent, or the first heterologous nucleic acid sequence encodes the second detectable agent and the second heterologous nucleic acid sequence is the reverse complement of a sequence encoding the first detectable agent. In embodiments, the first heterologous nucleic acid sequence encodes a first detectable agent and the second heterologous nucleic acid sequence is the reverse complement of a sequence encoding a second detectable agent. In embodiments, the first heterologous nucleic acid sequence encodes the second detectable agent and the second heterologous nucleic acid sequence is the reverse complement of a sequence encoding the first detectable agent.

In embodiments, the first heterologous nucleic acid sequence encodes a therapeutic agent or a detectable agent. In embodiments, the first heterologous nucleic acid sequence encodes a therapeutic agent. In embodiments, the first heterologous nucleic acid sequence encodes a detectable agent. In embodiments, the second heterologous nucleic acid sequence is the reverse complement of a sequence encoding a therapeutic agent or detectable agent. In embodiments, the second heterologous nucleic acid sequence is the reverse complement of a sequence encoding a therapeutic agent. In embodiments, the second heterologous nucleic acid sequence is the reverse complement of a sequence encoding a detectable agent.

In embodiments, the first therapeutic agent and the second therapeutic agent are independently an RNA-guided DNA endonuclease, an RNA-guided RNA nuclease, a nuclease-deficient RNA-guided DNA endonuclease, a guide RNA (gRNA), a therapeutic protein, a detectable protein, or an antibody. In embodiments, the first therapeutic agent is an RNA-guided DNA endonuclease, an RNA-guided RNA nuclease, nuclease-deficient RNA-guided DNA endonuclease, a guide RNA (gRNA), a therapeutic protein, a detectable protein, or an antibody. In embodiments, the first therapeutic agent is an RNA-guided DNA endonuclease. In embodiments, the first therapeutic agent is an RNA-guided RNA nuclease. In embodiments, the first therapeutic agent is a nuclease-deficient RNA-guided DNA endonuclease. In embodiments, the first therapeutic agent is a guide RNA (gRNA). In embodiments, the first therapeutic agent is a therapeutic protein. In embodiments, the first therapeutic agent is a detectable protein. In embodiments, the first therapeutic agent is an antibody. In embodiments, the second therapeutic agent is an RNA-guided DNA endonuclease, an RNA-guided RNA nuclease, nuclease-deficient RNA-guided DNA endonuclease, a guide RNA (gRNA), a therapeutic protein, a detectable protein, or an antibody. In embodiments, the second therapeutic agent is an RNA-guided DNA endonuclease. In embodiments, the second therapeutic agent is an RNA-guided RNA nuclease. In embodiments, the second therapeutic agent is an a nuclease-deficient RNA-guided DNA endonuclease. In embodiments, the second therapeutic agent is a guide RNA (gRNA). In embodiments, the second therapeutic agent is a therapeutic protein. In embodiments, the second therapeutic agent is a detectable protein. In embodiments, the second therapeutic agent is an antibody.

Applicant has demonstrated that the promoter (e.g. bidirectional promoter) provided herein including embodiments thereof can be used to deliver CRISPR systems (e.g. RNA-guided DNA endonuclease, gRNA, etc.). For example, the promoter (e.g. bidirectional promoter) provided herein is capable of expressing Cas12a or Cas9 in one direction (e.g. the DNA antisense strand), while one or more guide RNAs are expressed in the other direction (e.g. the DNA sense strand). Compared to unidirectional promoters known in the art, the promoters provided herein including embodiments thereof demonstrate increased expression levels of CRISPR system components (e.g. RNA-guided DNA endonuclease, gRNA, etc.). For example, when components of a CRISPR system are expressed from a unidirectional promoter generate a single transcript, certain Cas endonucleases (e.g. Cas9, Cas12a, etc.) are capable of cleaving their own mRNA transcripts, thereby lowering expression levels. In contrast, the promoter system provided herein is capable of generating two separate mRNA, thereby avoiding the potential problem of transcript cleavage.

Moreover, the promoters provided herein improve upon bidirectional promoters known in the art. For example, bidirectional promoters, such as the human HI promoter (described in, e.g., US Pat. Pub. US20160074535A1) that utilize Pol II expression from one end (e.g. in the sense direction) and Pol III expression from the other end (e.g. in the antisense direction) limit expression capability of the expression system (e.g. virus) to one protein and one relatively short gRNA. Without wishing to be bound by scientific theory, Pol III promoters are typically suitable for expression of short transcripts, thereby preventing the Pol III promoter from effectively expressing multiple gRNAs (for example, for multiplexed gene editing). In contrast, the promoter provided herein including embodiments thereof utilize Pol II expression from both ends, thereby allowing expression of two proteins or one protein and a plurality of gRNAs. Furthermore, the promoters provided herein including embodiments are capable of high expression levels of heterologous nucleic acid sequences in neurons (e.g. retinal neurons). Other bidirectional promoters (e.g. the CAG promoter) known in the art are capable of gene expression in neurons; however, the promoters provided herein are ⅛ the length of these promoters and display comparable expression levels.

In embodiments, the first therapeutic agent an RNA-guided DNA endonuclease and the second therapeutic agent is a gRNA, or the first therapeutic agent is the gRNA and the second therapeutic agent is the RNA-guided DNA endonuclease. In embodiments, the first therapeutic agent an RNA-guided DNA endonuclease and the second therapeutic agent is a gRNA. In embodiments, the first therapeutic agent is the gRNA and the second therapeutic agent is the RNA-guided DNA endonuclease. In embodiments, the RNA-guided DNA endonuclease is Cas9, Cas12a (Cpf1), a Class II CRISPR endonuclease, or variants thereof. In embodiments, the RNA-guided DNA endonuclease is Cas9 or a variant thereof. In embodiments, the RNA-guided DNA endonuclease is Cas12a (Cpf1) or a variant thereof, e.g., enAsCas12a (Kleinstiver, et al, 2019, Nat Biotechnol. 37 (3): 276-282). In embodiments, the RNA-guided DNA endonuclease is a Class II CRISPR endonuclease or a variant thereof.

In embodiments, the first therapeutic agent is a nuclease-deficient RNA-guided DNA endonuclease and the second therapeutic agent is a gRNA, or the first therapeutic agent is the gRNA and the second therapeutic agent is the a nuclease-deficient RNA-guided DNA endonuclease. In embodiments, the first therapeutic agent is a nuclease-deficient RNA-guided DNA endonuclease and the second therapeutic agent is a gRNA. In embodiments, the first therapeutic agent is the gRNA and the second therapeutic agent is the nuclease-deficient RNA-guided DNA endonuclease. In embodiments, the nuclease-deficient RNA-guided DNA endonuclease is dCas9, dCpf1 or ddCpf1. In embodiments, the nuclease-deficient RNA-guided DNA endonuclease is dCas9. In embodiments, the nuclease-deficient RNA-guided DNA endonuclease is dCpf1. In embodiments, the nuclease-deficient RNA-guided DNA endonuclease is ddCpf1.

The nuclease-deficient RNA-guided DNA endonuclease may be fused to one or more transcriptional activators or repressors, thereby allowing modulation of the expression level of one or more target genes. Thus, in embodiments, the nuclease-deficient RNA-guided DNA endonuclease is fused to a transcriptional activator or a transcriptional repressor. The term “transcriptional repressor” refers to a protein that decreases gene transcription of a gene or set of genes. The term “transcriptional activator” refers to a protein that increases gene transcription of a gene or set of genes. In embodiments, the nuclease-deficient RNA-guided DNA endonuclease is fused to a transcriptional repressor. In embodiments, the transcriptional repressor is a KRAB domain or a DNMT domain. In embodiments, the transcriptional repressor is a KRAB domain. In embodiments, the transcriptional repressor is a DNMT domain. In embodiments, the nuclease-deficient RNA-guided DNA endonuclease is fused to a transcriptional activator. In embodiments, the transcriptional activator is VP64, p65, Rta, or a combination of two or more thereof. In embodiments, the transcriptional activator is VP64. In embodiments, the transcriptional activator is p65. In embodiments, the transcriptional activator is Rta.

In embodiments, the first therapeutic agent an RNA-guided RNA nuclease and the second therapeutic agent is a gRNA, or the first therapeutic agent is the gRNA and the second therapeutic agent is the RNA-guided RNA nuclease. In embodiments, the first therapeutic agent an RNA-guided RNA nuclease and the second therapeutic agent is a gRNA. In embodiments, the first therapeutic agent is the gRNA and the second therapeutic agent is the RNA-guided RNA nuclease. In embodiments, RNA-guided RNA nuclease is Cas13.

In embodiments, the gRNA includes a plurality of gRNAs. For example, the gRNA may include 1, 2, 3, 4, 5 or more gRNAs, wherein each gRNA has a different target sequence.

For the virus provided herein, in embodiments, the first detectable agent and the second detectable agent are independently a detectable protein. In embodiments, the first detectable agent is a detectable protein. In embodiments, the second detectable agent is a detectable protein. In embodiments, the detectable protein is GFP, eGFP, Scarlet, or luciferase. In embodiments, the detectable protein is eGFP. In embodiments, the detectable protein is Scarlet. In embodiments, the detectable protein is luciferase

Applicant has further discovered that the virus composition provided herein may be used to express therapeutic agents that inhibit neuronal cell death. For example, therapeutic agents may include peptides that improve survival of neurons (e.g. RGCs). Therapeutic agents may include dominant negative SARM1, dominant negative DLK, and dominant negative LZK peptides. Dominant negative DLK and dominant negative LZK and methods for producing the same are in described in detail in WO2020/168111; and Xu, Z et al. “The MLK family mediates c-Jun N-terminal kinase activation in neuronal apoptosis.” Molecular and cellular biology vol. 21, 14 (2001): 4713-24. doi:10.1128/MCB.21.14.4713-4724.2001, which are incorporated herein in their entirety and for all purposes.

In embodiments, the first therapeutic agent is dominant negative SARM1 and the second therapeutic agent is dominant negative DLK. In embodiments, the first therapeutic agent is dominant negative DLK and the second therapeutic agent is dominant negative SARM1. Dominant negative SARM1 and methods for producing the same are described in: WO2019079572; and Geisler, Stefanie et al., Gene therapy targeting SARM1 blocks pathological axon degeneration in mice. The Journal of Experimental Medicine vol. 216, 2 (2019): 294-303. doi:10.1084/jem.20181040, which are incorporated herein in their entirety and for all purposes.

In embodiments, the first therapeutic agent is cytoplasmic NMNAT1 or a fragment thereof and the second therapeutic agent is dominant negative SARM1 In embodiments, the first therapeutic agent is dominant negative SARM1 and the second therapeutic agent is cytoplasmic NMNAT1 or a fragment thereof. In embodiments, the cytoplasmic NMNAT1 includes one or more mutations in its nuclear localization signal. NMNAT1 and methods for producing the same are known in the art and are described in detail in Babetto et al., 2010, “PTargeting NMNAT1 to axons and synapses transforms its neuroprotective potency in vivo” J Neurosci 30 (40): 13291-304); and Berger, et al. Subcellular Compartmentation and Differential Catalytic properties of Three Human Nicotinamide Mononuclease Adenyltransferase Isoforms. J Biol Chem 280 (43), 36334-36341, 2005., which are incorporated herein in their entirety and for all purposes. NMNAT1 including mutations in the nuclear localization signal and methods for producing the same are described in detail in Sasaki et al, Stimulation of Nicotinamide Adenine Dinucleotide Biosynthetic Pathways Delays Axonal Degeneration after Axotomy. J. Neurosci 26:8484-8491, 2006., which is incorporated herein in its entirety and for all purposes.

In embodiments, the first therapeutic agent is cytoplasmic NMNAT1 or a fragment thereof and the second therapeutic agent is dominant negative LZK. In embodiments, the first therapeutic agent is dominant negative LZK and the second therapeutic agent is cytoplasmic NMNAT1 or a fragment thereof.

In embodiments, the first therapeutic agent is NMNAT2 and the second therapeutic agent is dominant negative DLK. In embodiments, the first therapeutic agent is dominant negative DLK and the second therapeutic agent is NMNAT2. In embodiments, the first therapeutic agent is dominant negative DLK and the second therapeutic agent is NMNAT2 or an NMNAT2 peptide including a deletion in the subcellular targeting domain. In embodiments, the first therapeutic agent is NMNAT2 or an NMNAT2 peptide including a deletion in the subcellular targeting domain and the second therapeutic agent is dominant negative DLK.

In embodiments, the first therapeutic agent is NMNAT2 and the second therapeutic agent is dominant negative LZK. In embodiments, the first therapeutic agent is dominant negative LZK and the second therapeutic agent is NMNAT2. In embodiments, the first therapeutic agent is dominant negative LZK and the second therapeutic agent is NMNAT2 or an NMNAT2 peptide including a deletion in the subcellular targeting domain. In embodiments, the first therapeutic agent is NMNAT2 or an NMNAT2 peptide including a deletion in the subcellular targeting domain and the second therapeutic agent is dominant negative LZK.

In embodiments, the first therapeutic agent is dominant negative DLK or dominant negative LZK and the second therapeutic agent is osteopontin. In embodiments, the first therapeutic agent is osteopontin and the second therapeutic agent is dominant negative DLK or dominant negative LZK. See, for example, Chen et al., 2011, Osteopontin reduced hypoxia-ischemia neonatal brain injury by suppression of apoptosis in a rat pup model. Stroke 42 (3): 764-769)., which is incorporated herein in its entirety and for all purposes.

In embodiments, the first therapeutic agent is dominant negative DLK or dominant negative LZK and the second therapeutic agent is glucagon-like peptide-1 or brain derived neurotrophic factor. In embodiments, the first therapeutic agent is dominant negative DLK and the second therapeutic agent is glucagon-like peptide-1. In embodiments, the first therapeutic agent is glucagon-like peptide-1 and the second therapeutic agent is dominant negative DLK. In embodiments, the first therapeutic agent is dominant negative LZK and the second therapeutic agent is glucagon-like peptide-1. In embodiments, the first therapeutic agent is glucagon-like peptide-1 and the second therapeutic agent is dominant negative LZK. In embodiments, the first therapeutic agent is dominant negative DLK and the second therapeutic agent is brain derived neurotrophic factor. In embodiments, the first therapeutic agent is brain derived neurotrophic factor and the second therapeutic agent is dominant negative DLK. In embodiments, the first therapeutic agent is dominant negative LZK and the second therapeutic agent is brain derived neurotrophic factor. In embodiments, the first therapeutic agent is brain derived neurotrophic factor and the second therapeutic agent is dominant negative LZK. Glucagon-like peptide-1 and methods for producing the same are described in Holscher, C. Potential Role of Glucagon-like Peptide-1 (GLP-1) in Neuroprotection. CNS Drugs 26, 871-882 (2012); Velmurugan et al., Neuroprotective actions of Glucagon-like peptide-1 in differentiated human neuroprogenitor cells. J. Neurochem. 123 (6): 919-931 (2012); and WO2009039964A2, which are incorporated herein in their entirety and for all purposes. Brain-derived neurotrophic factor and methods for producing the same are described in Osborne, A et al. Neuroprotection of retinal ganglion cells by a novel gene therapy construct that achieves sustained enhancement of brain-derived neurotrophic factor/tropomyosin-related kinase receptor-B signaling. Cell Death Dis 9, 1007 (2018). https://doi.org/10.1038/s41419-018-1041-8., which is incorporated herein in its entirety and for all purposes.

Further examples of therapeutic agents that can be expressed with therapeutic dnDLK or dnDLK/LZK include the slow Wallerian degeneration polypeptide (Wld^{s}), dominant negative Rho-kinase, or a matrix metalloprotease (MMP) such as MMP-3 or MMP-1. Wld^{s}, Rho-kinase, and MMP are described in Conforti P., et al. Faulty neuronal determination and cell polarization are reverted by modulating HD early phenotypes. Proc Natl Acad Sci USA. 2018 Jan. 23; 115 (4); Amano M, Chihara K, Nakamura N, Kaneko T, Matsuura Y, Kaibuchi K. The COOH terminus of Rho-kinase negatively regulates Rho-kinase activity. J Biol Chem 1999; 274:32418-24; O'Callaghan J, Crosbie D E, Cassidy P S, Sherwood J M, Flügel-Koch C, Lütjen-Drecoll E, Humphries M M, Reina-Torres E, Wallace D, Kiang A S, Campbell M, Stamer W D, Overby D R, O'Brien C, Tam L C S, Humphries P. Therapeutic potential of AAV-mediated MMP-3 secretion from corneal endothelium in treating glaucoma. Hum Mol Genet. 2017 Apr. 1; 26 (7): 1230-1246. doi:10.1093/hmg/ddx028. PMID: 28158775; PMCID: PMC5390678; and Borrás T, Buie L K, Spiga M G Inducible scAAV2.GRE.MMP1 lowers IOP long-term in a large animal model for steroid-induced glaucoma gene therapy. Gene Ther. 2016, 23:438-49. https://doi.org/10.1038/gt.2016.14., which are incorporated herein in their entirety and for all purposes.

Additional examples of therapeutic agents capable of neuroprotection include SCG10/STMN2, BCL-XL, or TRKB; an aquaporin, CNTF, BDNF, GDNF, NGF, a complement inhibitor, GLP-1R or GLP-1, CDKN2B-AS1, GLDN, CHL1, QPCT, TBX20, DGKG, TIMP2, EGR1, EOMES, JUNB, IGFBP2, OSTF1, FGF1, SEMA5A, ESRRG, KBTBD11, RAMP1, ETL4, PRKCQ, CTXN3, NDNF, MAN1A, SDK2, PRPH, SDK1, or IFI27.

In embodiments, a first heterologous nucleic acid sequence (e.g. encoding dnDLK or dnDLK/LZK) is expressed with a second heterologous nucleic acid sequence encoding a therapeutic agent that inhibits expression of a gene, or the activity of a product encoded by the gene to enhance neural protection. In embodiments, the therapeutic agent is a nucleic acid, for example, an antisense RNA or shRNA that targets the gene to be inhibited. In embodiments, the therapeutic agent targets MEKK4, MLK2/MAP3K10, PUMA/BBC3, SARM1, ROCK1, ROCK2, TAOK1, TAOK2, TAOK3, TNIK, MAP4K4, MINK1, GSK-3β, GSK-3α, MAP2K7/MKK7, MAP2K4/MKK4, PERK, CHOP, HSP90, SNRK, JNK1 (MAPK8), JNK2 (MAPK9), JNK3 (MAPK10), JUN, ATF2, MEF2A, SOX11, MST1/STK4, MST2/STK3, END1, or END2.

In embodiments, the promoter is from about 150 to about 300 nucleotides in length. In embodiments, the promoter is from about 160 to about 300 nucleotides in length. In embodiments, the promoter is from about 170 to about 300 nucleotides in length. In embodiments, the promoter is from about 180 to about 300 nucleotides in length. In embodiments, the promoter is from about 190 to about 300 nucleotides in length. In embodiments, the promoter is from about 200 to about 300 nucleotides in length. In embodiments, the promoter is from about 210 to about 300 nucleotides in length. In embodiments, the promoter is from about 220 to about 300 nucleotides in length. In embodiments, the promoter is from about 230 to about 300 nucleotides in length. In embodiments, the promoter is from about 240 to about 300 nucleotides in length. In embodiments, the promoter is from about 250 to about 300 nucleotides in length. In embodiments, the promoter is from about 260 to about 300 nucleotides in length. In embodiments, the promoter is from about 270 to about 300 nucleotides in length. In embodiments, the promoter is from about 280 to about 300 nucleotides in length. In embodiments, the promoter is from about 290 to about 300 nucleotides in length.

In embodiments, the promoter is from about 150 to about 290 nucleotides in length. In embodiments, the promoter is from about 150 to about 280 nucleotides in length. In embodiments, the promoter is from about 150 to about 270 nucleotides in length. In embodiments, the promoter is from about 150 to about 260 nucleotides in length. In embodiments, the promoter is from about 150 to about 250 nucleotides in length. In embodiments, the promoter is from about 150 to about 240 nucleotides in length. In embodiments, the promoter is from about 150 to about 230 nucleotides in length. In embodiments, the promoter is from about 150 to about 220 nucleotides in length. In embodiments, the promoter is from about 150 to about 210 nucleotides in length. In embodiments, the promoter is from about 150 to about 200 nucleotides in length. In embodiments, the promoter is from about 150 to about 190 nucleotides in length. In embodiments, the promoter is from about 150 to about 180 nucleotides in length. In embodiments, the promoter is from about 150 to about 170 nucleotides in length. In embodiments, the promoter is from about 150 to about 160 nucleotides in length. In embodiments, the promoter is about 150, 160, 170, 180, 190, 200, 210, 220, 230, 240, 250, 260, 270, 280, 290, or 300 nucleotides in length.

In embodiments, the promoter is from about 200 to about 250 nucleotides in length. In embodiments, the promoter is from about 205 to about 250 nucleotides in length. In embodiments, the promoter is from about 210 to about 250 nucleotides in length. In embodiments, the promoter is from about 215 to about 250 nucleotides in length. In embodiments, the promoter is from about 220 to about 250 nucleotides in length. In embodiments, the promoter is from about 225 to about 250 nucleotides in length. In embodiments, the promoter is from about 230 to about 250 nucleotides in length. In embodiments, the promoter is from about 235 to about 250 nucleotides in length. In embodiments, the promoter is from about 240 to about 250 nucleotides in length. In embodiments, the promoter is from about 245 to about 250 nucleotides in length.

In embodiments, the promoter is from about 200 to about 245 nucleotides in length. In embodiments, the promoter is from about 200 to about 240 nucleotides in length. In embodiments, the promoter is from about 205 to about 235 nucleotides in length. In embodiments, the promoter is from about 205 to about 230 nucleotides in length. In embodiments, the promoter is from about 205 to about 225 nucleotides in length. In embodiments, the promoter is from about 205 to about 220 nucleotides in length. In embodiments, the promoter is from about 205 to about 215 nucleotides in length. In embodiments, the promoter is from about 205 to about 210 nucleotides in length. In embodiments, the promoter is about 205, 210, 215, 220, 225, 230, 235, 240, 245, or 250 nucleotides in length. In embodiments, the promoter is about 205 nucleotides in length. In embodiments, the promoter is 205 nucleotides in length. In embodiments, the promoter is about 235 nucleotides in length. In embodiments, the promoter is 235 nucleotides in length.

Promoters used in the methods described herein have bidirectional promoter activity, as described below and in the examples. In embodiments, a promoter (e.g. bidirectional promoter) used as disclosed herein has a sequence with at least 80% identity to SEQ ID NO: 10 (human Promoter 10), SEQ ID NO:25 (mouse Promoter 10) or SEQ ID NO:27 (consensus Promoter 10), each of which may be referred to as a “Promoter 10 reference sequence”. In embodiments, a promoter (e.g. bidirectional promoter) used as disclosed herein has a sequence with at least 80% identity to SEQ ID NO:21. As shown in Example 4, Promoter 10 can be truncated at the TMEM208 side and retain bidirectional promoter activity. Short truncations at the LRRC29 side, generally less than about 67 bases may also retain activity. In embodiments the truncations at either end of the promoter (e.g. 3′ end or 5′ end) may be, independently and without limitation, 1-10 base pairs, 10-20 base pairs, 15-25 base pairs, 20 to 35 base pairs, 25 to 40 base pairs, 30 to 40 base pairs, 35 to 45 base pairs, 40 to 50 base pairs, 45 to 50 base pairs, or longer). Accordingly, fragments of SEQ ID NO: NO: 10, SEQ ID NO:25 or SEQ ID NO:27 may be used as bidirectional promoters. In addition, promoter activity can tolerate a certain degree of sequence variation relative to a reference sequence and variants of a reference sequence or of a fragment of a reference sequence may be used as bidirectional promoters, including but not limited to short deletions or insertions (e.g., less than 10 bases, or less than 20 bases).

Furthermore, human Promoter 10 sequence (SEQ ID NO:10) was evaluated using the JASPER database to identify consensus sequences for transcription factor binding sites. Referring to the sequence numbering of SEQ ID NO: 10, the following sequence were identified. According to the Broad single cell portal E2f4, TFAP2C, PRDM9, EGR1, STAT3, THAP11, ZKSCAN5, FL2, ELK4, and STAT5b are expressed in mouse adult RGCs (See Tran et al, Neuron 104:1039-1055 E12, 2019). Without intending to be bound by any specific theory or mechanism, one or more of domains may function as TF binding sites in mouse and human cells in which a construct of the present invention is expressed. Accordingly, in some embodiments one or more of these sequences, independently selected, may be retained in Promoter 10 variants and fragments described herein.

TABLE 1
Transcription factors and respective binding sites

| Transcription | Binding | Transcription | Binding |
|---|---|---|---|
| Factor | site | Factor | site |
| E2f4 | 1 . . . 138 | STAT3 | 103 . . . 113 |
| TFAP2C | 18 . . . 32 | THAP11 | 106-119 |
| TFAP2C | 38 . . . 52 | ZKSCAN5 | 145 . . . 158 |
| PRDM9 | 70 . . . 93 | FLI1 | 195-205 |
| EGR1 | 80 . . . 93 | ELK4 | 195-205 |
| ZNF76 | 94 . . . 114 | STAT5b | 101 . . . 115 |

A promoter fragment, truncation or variant has bidirectional promoter activity in a specified cell type when the promoter drives expression of two transgenes (e.g., EGFP and Scarlet) and produces at least 50% of the fluorescent intensity of each transgene compared to the florescent intensity produced using SEQ ID NO:10. In embodiments, a fragment, truncation or variant has bidirectional promoter activity when it produces at least 75%, or at least 100%, of the fluorescent intensity of each transgene as is produced using SEQ ID NO:10. In embodiments, the assay is carried out positioning mScarlet (or the corresponding fragment or variant) at the TMEM208 end of the promoter and EGFP (or the corresponding fragment or variant) at the LRRC29 end of the promoter.

In embodiments, the 5′ terminus (e.g. 5′ end) of a first transgene on one DNA strand and the complement of the 5′ terminus (e.g. 5′ end) of the other transgene (encoded on the other DNA strand (e.g. the complementary strand)) are separated by a distance of 100 to 300 nucleotides, often 125 to 300 nucleotides often 150 to 300 nucleotides, often 175 to 300 nucleotides, often 175 to 240 nucleotides, and sometimes 180 to 235 nucleotides.

In embodiments, the virus has a genome including a promoter having at least 80%, 85%, 90%, 95%, 96%, 97%, 98%, 99% or 100% sequence identity across the whole sequence or a portion of the sequence (e.g. a portion comprising at least 20, 30, or 40 continuous bases) compared to SEQ ID NO: 10. In embodiments, the promoter has at least 50%, 80%, 90%, 95%, 96%, 97%, 98%, 99% or 100% activity compared to SEQ ID NO:10. Activity can be assessed, for example, by measuring expression levels of the heterologous nucleic acid sequence operably linked to the promoter, for example by qPCR, Western Blot, etc. or any other method known in the biological arts. Expression levels may be measured by detecting the function or activity of a therapeutic or detectable agent encoded by the heterologous nucleic acid sequence operably linked to the promoter. In embodiments, the virus has a genome including a promoter having at least 80% sequence identity to the sequence of SEQ ID NO: 10. In embodiments, the virus has a genome including a promoter having at least 81% sequence identity to the sequence of SEQ ID NO: 10. In embodiments, the virus has a genome including a promoter having at least 82% sequence identity to the sequence of SEQ ID NO: 10. In embodiments, the virus has a genome including a promoter having at least 83% sequence identity to the sequence of SEQ ID NO: 10. In embodiments, the virus has a genome including a promoter having at least 84% sequence identity to the sequence of SEQ ID NO:10. In embodiments, the virus has a genome including a promoter having at least 85% sequence identity to the sequence of SEQ ID NO: 10. In embodiments, the virus has a genome including a promoter having at least 86% sequence identity to the sequence of SEQ ID NO: 10. In embodiments, the virus has a genome including a promoter having at least 87% sequence identity to the sequence of SEQ ID NO: 10. In embodiments, the virus has a genome including a promoter having at least 88% sequence identity to the sequence of SEQ ID NO: 10. In embodiments, the virus has a genome including a promoter having at least 89% sequence identity to the sequence of SEQ ID NO:10. In embodiments, the virus has a genome including a promoter having at least 90% sequence identity to the sequence of SEQ ID NO: 10. In embodiments, the virus has a genome including a promoter having at least 91% sequence identity to the sequence of SEQ ID NO:10. In embodiments, the virus has a genome including a promoter having at least 92% sequence identity to the sequence of SEQ ID NO: 10. In embodiments, the virus has a genome including a promoter having at least 93% sequence identity to the sequence of SEQ ID NO: 10. In embodiments, the virus has a genome including a promoter having at least 94% sequence identity to the sequence of SEQ ID NO:10. In embodiments, the virus has a genome including a promoter having at least 95% sequence identity to the sequence of SEQ ID NO:10. In embodiments, the virus has a genome including a promoter having at least 96% sequence identity to the sequence of SEQ ID NO:10. In embodiments, the virus has a genome including a promoter having at least 97% sequence identity to the sequence of SEQ ID NO: 10. In embodiments, the virus has a genome including a promoter having at least 98% sequence identity to the sequence of SEQ ID NO: 10. In embodiments, the virus has a genome including a promoter having at least 99% sequence identity to the sequence of SEQ ID NO:10.

In embodiments, the virus has a genome including a promoter having about 80% sequence identity to the sequence of SEQ ID NO: 10. In embodiments, the virus has a genome including a promoter having about 81% sequence identity to the sequence of SEQ ID NO: 10. In embodiments, the virus has a genome including a promoter having about 82% sequence identity to the sequence of SEQ ID NO: 10. In embodiments, the virus has a genome including a promoter having about 83% sequence identity to the sequence of SEQ ID NO: 10. In embodiments, the virus has a genome including a promoter having about 84% sequence identity to the sequence of SEQ ID NO:10. In embodiments, the virus has a genome including a promoter having about 85% sequence identity to the sequence of SEQ ID NO: 10. In embodiments, the virus has a genome including a promoter having about 86% sequence identity to the sequence of SEQ ID NO:10. In embodiments, the virus has a genome including a promoter having about 87% sequence identity to the sequence of SEQ ID NO: 10. In embodiments, the virus has a genome including a promoter having about 88% sequence identity to the sequence of SEQ ID NO: 10. In embodiments, the virus has a genome including a promoter having about 89% sequence identity to the sequence of SEQ ID NO:10. In embodiments, the virus has a genome including a promoter having about 90% sequence identity to the sequence of SEQ ID NO: 10. In embodiments, the virus has a genome including a promoter having about 91% sequence identity to the sequence of SEQ ID NO: 10. In embodiments, the virus has a genome including a promoter having about 92% sequence identity to the sequence of SEQ ID NO: 10. In embodiments, the virus has a genome including a promoter having about 93% sequence identity to the sequence of SEQ ID NO: 10. In embodiments, the virus has a genome including a promoter having about 94% sequence identity to the sequence of SEQ ID NO:10. In embodiments, the virus has a genome including a promoter having about 95% sequence identity to the sequence of SEQ ID NO: 10. In embodiments, the virus has a genome including a promoter having about 96% sequence identity to the sequence of SEQ ID NO: 10. In embodiments, the virus has a genome including a promoter having about 97% sequence identity to the sequence of SEQ ID NO: 10. In embodiments, the virus has a genome including a promoter having about 98% sequence identity to the sequence of SEQ ID NO: 10. In embodiments, the virus has a genome including a promoter having about 99% sequence identity to the sequence of SEQ ID NO:10. In embodiments, the promoter includes the sequence of SEQ ID NO: 10. In embodiments, the promoter is the sequence of SEQ ID NO:10.

In embodiments, the promoter has at least 80% sequence identity, at least 85%, at least 90%, at least 95% sequence identity, or at least 98% sequence identity to SEQ ID NO:10, wherein the sequence is not SEQ ID NO: 10. In embodiments, the promoter does not include the sequence of SEQ ID NO: 10.

In embodiments, the virus has a genome including a promoter having at least 80%, 85%, 90%, 95%, 96%, 97%, 98%, 99% or 100% sequence identity across the whole sequence or a portion of the sequence (e.g. a portion comprising at least 20, 30, or 40 continuous bases) compared to SEQ ID NO:21. In embodiments, the promoter has at least 50%, 80%, 90%, 95%, 96%, 97%, 98%, 99% or 100% activity compared to SEQ ID NO:21. In embodiments, the virus has a genome including a promoter having at least 80% sequence identity to the sequence of SEQ ID NO:21. In embodiments, the virus has a genome including a promoter having at least 81% sequence identity to the sequence of SEQ ID NO:21. In embodiments, the virus has a genome including a promoter having at least 82% sequence identity to the sequence of SEQ ID NO: 21. In embodiments, the virus has a genome including a promoter having at least 83% sequence identity to the sequence of SEQ ID NO:21. In embodiments, the virus has a genome including a promoter having at least 84% sequence identity to the sequence of SEQ ID NO:21. In embodiments, the virus has a genome including a promoter having at least 85% sequence identity to the sequence of SEQ ID NO:21. In embodiments, the virus has a genome including a promoter having at least 86% sequence identity to the sequence of SEQ ID NO:21. In embodiments, the virus has a genome including a promoter having at least 87% sequence identity to the sequence of SEQ ID NO:21. In embodiments, the virus has a genome including a promoter having at least 88% sequence identity to the sequence of SEQ ID NO:21. In embodiments, the virus has a genome including a promoter having at least 89% sequence identity to the sequence of SEQ ID NO:21. In embodiments, the virus has a genome including a promoter having at least 90% sequence identity to the sequence of SEQ ID NO:21. In embodiments, the virus has a genome including a promoter having at least 91% sequence identity to the sequence of SEQ ID NO:21. In embodiments, the virus has a genome including a promoter having at least 92% sequence identity to the sequence of SEQ ID NO:21. In embodiments, the virus has a genome including a promoter having at least 93% sequence identity to the sequence of SEQ ID NO:21. In embodiments, the virus has a genome including a promoter having at least 94% sequence identity to the sequence of SEQ ID NO:21. In embodiments, the virus has a genome including a promoter having at least 95% sequence identity to the sequence of SEQ ID NO:21. In embodiments, the virus has a genome including a promoter having at least 96% sequence identity to the sequence of SEQ ID NO:21. In embodiments, the virus has a genome including a promoter having at least 97% sequence identity to the sequence of SEQ ID NO:21. In embodiments, the virus has a genome including a promoter having at least 98% sequence identity to the sequence of SEQ ID NO:21. In embodiments, the virus has a genome including a promoter having at least 99% sequence identity to the sequence of SEQ ID NO:21.

In embodiments, the virus has a genome including a promoter having about 80% sequence identity to the sequence of SEQ ID NO:21. In embodiments, the virus has a genome including a promoter having about 81% sequence identity to the sequence of SEQ ID NO:21. In embodiments, the virus has a genome including a promoter having about 82% sequence identity to the sequence of SEQ ID NO:21. In embodiments, the virus has a genome including a promoter having about 83% sequence identity to the sequence of SEQ ID NO:21. In embodiments, the virus has a genome including a promoter having about 84% sequence identity to the sequence of SEQ ID NO:21. In embodiments, the virus has a genome including a promoter having about 85% sequence identity to the sequence of SEQ ID NO:21. In embodiments, the virus has a genome including a promoter having about 86% sequence identity to the sequence of SEQ ID NO:21. In embodiments, the virus has a genome including a promoter having about 87% sequence identity to the sequence of SEQ ID NO:21. In embodiments, the virus has a genome including a promoter having about 88% sequence identity to the sequence of SEQ ID NO:21. In embodiments, the virus has a genome including a promoter having about 89% sequence identity to the sequence of SEQ ID NO:21. In embodiments, the virus has a genome including a promoter having about 90% sequence identity to the sequence of SEQ ID NO:21. In embodiments, the virus has a genome including a promoter having about 91% sequence identity to the sequence of SEQ ID NO:21. In embodiments, the virus has a genome including a promoter having about 92% sequence identity to the sequence of SEQ ID NO:21. In embodiments, the virus has a genome including a promoter having about 93% sequence identity to the sequence of SEQ ID NO:21. In embodiments, the virus has a genome including a promoter having about 94% sequence identity to the sequence of SEQ ID NO:21. In embodiments, the virus has a genome including a promoter having about 95% sequence identity to the sequence of SEQ ID NO:21. In embodiments, the virus has a genome including a promoter having about 96% sequence identity to the sequence of SEQ ID NO:21. In embodiments, the virus has a genome including a promoter having about 97% sequence identity to the sequence of SEQ ID NO:21. In embodiments, the virus has a genome including a promoter having about 98% sequence identity to the sequence of SEQ ID NO:21. In embodiments, the virus has a genome including a promoter having about 99% sequence identity to the sequence of SEQ ID NO:21. In embodiments, the promoter includes the sequence of SEQ ID NO:21. In embodiments, the promoter is the sequence of SEQ ID NO:21.

In embodiments, the promoter includes a fragment or portion of SEQ ID NO:10. In embodiments, the promoter includes the nucleotide sequence of the LLRC29 side of the Promoter 10 sequence (SEQ ID NO: 10) or the nucleotide sequence of the LLRC29 side of the mouse Promoter 10 ortholog sequence (SEQ ID NO:25).

Applicant has discovered that including one or more intron sequences in the viral genome may increase expression of the therapeutic and/or detectable agent encoded by the first and/or sequence heterologous nucleic acid sequence. The term “intron” is used in accordance to its plain ordinary meaning and refers to a non-coding nucleic acid sequence. Typically, when found in nature, an intron is located in a region within a gene. An intron can be a DNA sequence or the corresponding RNA transcript. In nature, introns are typically removed during RNA processing pathways through RNA splicing, which takes place following transcription and prior to translation. In the present invention, the intron (e.g. intronic sequence) is fused to a location within the virus genome. The intron may be attached to the first heterologous nucleic acid sequence or the second nucleic acid sequence. The intron may be attached to the 5′ end or 3′ end of the promoter.

In embodiments, the intron is heterologous to the virus provided herein. In embodiments, the viral genome includes one or more introns. For example, the genome provided herein may include 1, 2, 3, 4 or more introns. In embodiments, the intron links the promoter to the first heterologous nucleic acid sequence, or links the promoter to the second heterologous nucleic acid sequence. In embodiments, the intron links the promoter to the first heterologous nucleic acid sequence. Thus, in this instance, the intron is attached to the 3′ end of the promoter. In embodiments, the intron links the promoter to the second heterologous nucleic acid sequence. In this instance, the intron is attached to the 5′ end of the promoter.

In embodiments, the intron has at least 80%, 85%, 90%, 95%, 96%, 97%, 98%, 99% or 100% sequence identity across the whole sequence or a portion of the sequence (e.g. a portion comprising at least 20, 30, or 40 continuous bases) compared to SEQ ID NO:22. In embodiments, the intron comprises a sequence having at least 80% sequence identity to the SEQ ID NO: 22. In embodiments, the intron comprises a sequence having at least 81% sequence identity to the SEQ ID NO:22. In embodiments, the intron comprises a sequence having at least 82% sequence identity to the SEQ ID NO:22. In embodiments, the intron comprises a sequence having at least 83% sequence identity to the SEQ ID NO:22. In embodiments, the intron comprises a sequence having at least 84% sequence identity to the SEQ ID NO:22. In embodiments, the intron comprises a sequence having at least 85% sequence identity to the SEQ ID NO: 22. In embodiments, the intron comprises a sequence having at least 86% sequence identity to the SEQ ID NO:22. In embodiments, the intron comprises a sequence having at least 87% sequence identity to the SEQ ID NO:22. In embodiments, the intron comprises a sequence having at least 88% sequence identity to the SEQ ID NO:22. In embodiments, the intron comprises a sequence having at least 89% sequence identity to the SEQ ID NO:22. In embodiments, the intron comprises a sequence having at least 90% sequence identity to the SEQ ID NO: 22. In embodiments, the intron comprises a sequence having at least 91% sequence identity to the SEQ ID NO:22. In embodiments, the intron comprises a sequence having at least 92% sequence identity to the SEQ ID NO:22. In embodiments, the intron comprises a sequence having at least 93% sequence identity to the SEQ ID NO:22. In embodiments, the intron comprises a sequence having at least 94% sequence identity to the SEQ ID NO:22. In embodiments, the intron comprises a sequence having at least 95% sequence identity to the SEQ ID NO: 22. In embodiments, the intron comprises a sequence having at least 96% sequence identity to the SEQ ID NO:22. In embodiments, the intron comprises a sequence having at least 97% sequence identity to the SEQ ID NO:22. In embodiments, the intron comprises a sequence having at least 98% sequence identity to the SEQ ID NO:22. In embodiments, the intron comprises a sequence having at least 99% sequence identity to the SEQ ID NO:22.

In embodiments, the intron comprises a sequence having about 80% sequence identity to the SEQ ID NO:22. In embodiments, the intron comprises a sequence having about 81% sequence identity to the SEQ ID NO:22. In embodiments, the intron comprises a sequence having about 82% sequence identity to the SEQ ID NO:22. In embodiments, the intron comprises a sequence having about 83% sequence identity to the SEQ ID NO:22. In embodiments, the intron comprises a sequence having about 84% sequence identity to the SEQ ID NO: 22. In embodiments, the intron comprises a sequence having about 85% sequence identity to the SEQ ID NO:22. In embodiments, the intron comprises a sequence having about 86% sequence identity to the SEQ ID NO:22. In embodiments, the intron comprises a sequence having about 87% sequence identity to the SEQ ID NO:22. In embodiments, the intron comprises a sequence having about 88% sequence identity to the SEQ ID NO:22. In embodiments, the intron comprises a sequence having about 89% sequence identity to the SEQ ID NO: 22. In embodiments, the intron comprises a sequence having about 90% sequence identity to the SEQ ID NO:22. In embodiments, the intron comprises a sequence having about 91% sequence identity to the SEQ ID NO:22. In embodiments, the intron comprises a sequence having about 92% sequence identity to the SEQ ID NO:22. In embodiments, the intron comprises a sequence having about 93% sequence identity to the SEQ ID NO:22. In embodiments, the intron comprises a sequence having about 94% sequence identity to the SEQ ID NO: 22. In embodiments, the intron comprises a sequence having about 95% sequence identity to the SEQ ID NO:22. In embodiments, the intron comprises a sequence having about 96% sequence identity to the SEQ ID NO:22. In embodiments, the intron comprises a sequence having about 97% sequence identity to the SEQ ID NO:22. In embodiments, the intron comprises a sequence having about 98% sequence identity to the SEQ ID NO:22. In embodiments, the intron comprises a sequence having about 99% sequence identity to the SEQ ID NO: 22. In embodiments, the intron includes the sequence of SEQ ID NO:22. In embodiments, the intron is the sequence of SEQ ID NO:22.

In embodiments, the intron has at least 80%, 85%, 90%, 95%, 96%, 97%, 98%, 99% or 100% sequence identity across the whole sequence or a portion of the sequence (e.g. a portion comprising at least 20, 30, or 40 continuous bases) compared to SEQ ID NO:23. In embodiments, the intron comprises a sequence having at least 80% sequence identity to the SEQ ID NO: 23. In embodiments, the intron comprises a sequence having at least 81% sequence identity to the SEQ ID NO:23. In embodiments, the intron comprises a sequence having at least 82% sequence identity to the SEQ ID NO:23. In embodiments, the intron comprises a sequence having at least 83% sequence identity to the SEQ ID NO:23. In embodiments, the intron comprises a sequence having at least 84% sequence identity to the SEQ ID NO:23. In embodiments, the intron comprises a sequence having at least 85% sequence identity to the SEQ ID NO: 23. In embodiments, the intron comprises a sequence having at least 86% sequence identity to the SEQ ID NO:23. In embodiments, the intron comprises a sequence having at least 87% sequence identity to the SEQ ID NO:23. In embodiments, the intron comprises a sequence having at least 88% sequence identity to the SEQ ID NO:23. In embodiments, the intron comprises a sequence having at least 89% sequence identity to the SEQ ID NO:23. In embodiments, the intron comprises a sequence having at least 90% sequence identity to the SEQ ID NO: 23. In embodiments, the intron comprises a sequence having at least 91% sequence identity to the SEQ ID NO:23. In embodiments, the intron comprises a sequence having at least 92% sequence identity to the SEQ ID NO:23. In embodiments, the intron comprises a sequence having at least 93% sequence identity to the SEQ ID NO:23. In embodiments, the intron comprises a sequence having at least 94% sequence identity to the SEQ ID NO:23. In embodiments, the intron comprises a sequence having at least 95% sequence identity to the SEQ ID NO: 23. In embodiments, the intron comprises a sequence having at least 96% sequence identity to the SEQ ID NO:23. In embodiments, the intron comprises a sequence having at least 97% sequence identity to the SEQ ID NO:23. In embodiments, the intron comprises a sequence having at least 98% sequence identity to the SEQ ID NO:23. In embodiments, the intron comprises a sequence having at least 99% sequence identity to the SEQ ID NO:23.

In embodiments, the intron comprises a sequence having about 80% sequence identity to the SEQ ID NO:23. In embodiments, the intron comprises a sequence having about 81% sequence identity to the SEQ ID NO:23. In embodiments, the intron comprises a sequence having about 82% sequence identity to the SEQ ID NO:23. In embodiments, the intron comprises a sequence having about 83% sequence identity to the SEQ ID NO:23. In embodiments, the intron comprises a sequence having about 84% sequence identity to the SEQ ID NO: 23. In embodiments, the intron comprises a sequence having about 85% sequence identity to the SEQ ID NO:23. In embodiments, the intron comprises a sequence having about 86% sequence identity to the SEQ ID NO:23. In embodiments, the intron comprises a sequence having about 87% sequence identity to the SEQ ID NO:23. In embodiments, the intron comprises a sequence having about 88% sequence identity to the SEQ ID NO:23. In embodiments, the intron comprises a sequence having about 89% sequence identity to the SEQ ID NO: 23. In embodiments, the intron comprises a sequence having about 90% sequence identity to the SEQ ID NO:23. In embodiments, the intron comprises a sequence having about 91% sequence identity to the SEQ ID NO:23. In embodiments, the intron comprises a sequence having about 92% sequence identity to the SEQ ID NO:23. In embodiments, the intron comprises a sequence having about 93% sequence identity to the SEQ ID NO:23. In embodiments, the intron comprises a sequence having about 94% sequence identity to the SEQ ID NO: 23. In embodiments, the intron comprises a sequence having about 95% sequence identity to the SEQ ID NO:23. In embodiments, the intron comprises a sequence having about 96% sequence identity to the SEQ ID NO:23. In embodiments, the intron comprises a sequence having about 97% sequence identity to the SEQ ID NO:23. In embodiments, the intron comprises a sequence having about 98% sequence identity to the SEQ ID NO:23. In embodiments, the intron comprises a sequence having about 99% sequence identity to the SEQ ID NO: 23. In embodiments, the intron includes the sequence of SEQ ID NO:23. In embodiments, the intron is the sequence of SEQ ID NO:23.

Applicant has further found that inclusion of one or more insulator sequences in the viral genome can increase expression of one or more of the therapeutic and or detectable agents provided herein. “Insulator” or “insulator sequence” refer to a short (e.g. 2 nucleotide to 20 nucleotide long) nucleic acid sequence that flank 5′ end or 3′ end of the first and/or second heterologous nucleic acid sequence. An insulator therefore provides a short linking element between the first and/or sequence heterologous nucleic acid sequence and an adjoining sequence. In embodiments, the genome includes one or more insulator sequences.

In embodiments, the insulator sequence is attached to the 5′ end or 3′ end of the first heterologous nucleic acid sequence or the second heterologous nucleic acid sequence. In embodiments, the insulator sequence is attached to the 5′ end of the first heterologous nucleic acid sequence. In embodiments, the insulator sequence is attached to the 5′ end of the second heterologous nucleic acid sequence. In embodiments, the insulator sequence is attached to 3′ end of the first heterologous nucleic acid sequence. In embodiments, the insulator sequence is attached to the 3′ end of the second heterologous nucleic acid sequence.

Applicant has discovered that inclusion of one or more insulator sequences between sequences encoding gRNAs increases expression of the gRNAs. Thus, the insulator sequence may link a plurality of heterologous nucleic acid sequences encoding gRNAs. In embodiments, the insulator sequence links a first nucleic acid sequence encoding a first gRNA and a second nucleic acid sequence encoding a second gRNA. In embodiments, the insulator sequence links a first, second, third, fourth, or more nucleic acid sequence encoding a plurality of gRNA. The insulator sequence may link a plurality of nucleic acid sequences wherein the sequences are the reverse complements of sequences encoding gRNAs. In embodiments, the insulator links the reverse complement of a sequence encoding a first gRNA and the reverse complement of a sequence encoding a second gRNA. In embodiments, the insulator sequence links a first, second, third, fourth, or more sequences, wherein the sequences are reverse complements of sequences encoding a plurality of gRNA.

In embodiments, the insulator sequence includes the sequence of SEQ ID NO:24. In embodiments, the insulator sequence is the sequence of SEQ ID NO:24.

In embodiments, the virus provided herein is an is a herpes simplex virus, an adeno-associated virus, a lentivirus, an adenovirus, a vaccinia virus, a poxvirus, an alphavirus, an enterovirus, a papillomavirus, or a poliovirus. In embodiments, the virus is an is a herpes simplex virus. In embodiments, the virus is an is adeno-associated virus (AAV). In embodiments, the virus is a lentivirus. In embodiments, the virus is an is an adenovirus. In embodiments, the virus is a vaccinia virus. In embodiments, the virus is an is a poxvirus. In embodiments, the virus is an is a alphavirus poliovirus. In embodiments, the virus is an is an enterovirus. In embodiments, the virus is an is an papillomavirus. In embodiments, the virus is an is a poliovirus.

Adeno-associated virus (AAV) is a small virus of approximately 20 nm, including a single-stranded DNA genome. AAV has a cargo capacity of about 4.7 kilobases, including the inverted terminal repeats (ITR). “Inverted terminal repeat” or “ITR” is used in accordance to its ordinary meaning in the art, and refers to a single-stranded nucleic acid sequence followed by the reverse complement of the nucleic acid sequence with an intervening sequence in between. The intervening sequence of nucleotides between the nucleic acid sequence and the reverse complement of the nucleic acid sequence of the IRT can be any length. For example, the intervening sequence can include the promoter provided herein and the first and second heterologous nucleic acid sequences provided herein. The AAV genome is typically flanked by ITRs, which can serve as self-priming structure for DNA replication. AAV includes a single-stranded DNA genome. Upon entry into the host cell, the second strand of the AAV genome may be synthesized, thereby allowing for gene transcription.

In embodiments, the AAV virus has a genome including an adeno-associated viral (AAV) vector. In embodiments, the AAV vector may selected from AAV1, AAV2, AAV3b, AAV4, AAV5, AAV6, AAV7, AAV8, AAV9, AAV10, AAV-11, AAV-12, AAV113, AAVrh.74, AAV2.7m8, AAV.ANC80, Anc80L65 and derivatives thereof, and AAVDJ, and combinations thereof. An extensive listing of illustrative AAV serotypes, including variants of naturally occurring AAV serotypes, is provided in U.S. Pat. No. 10,662,425, which is incorporated herein in its entirety and for all purposes. In embodiments, the AAV vector includes sequences including those of AAV that are required in cis for replication and packaging (e.g., functional ITRs) of the DNA into a virion. In embodiments, AAV vectors have one or more of the AAV WT genes deleted in whole or in part, but retain functional flanking ITR sequences.

In embodiments, an AAV (e.g. AAV virion or AAV particle) comprises an AAV capsid protein and an AAV vector. In some instances, the AAV is a hybrid or chimeric AAV, e.g., an AAV particle can comprise ITRs that are of a heterologous serotype in comparison with the capsid serotype (e.g., AAV2 ITRs with AAV5, AAV6, or AAV8 capsids). The AAV ITRs may be of any serotype suitable for a particular application.

For the virus provided herein, in embodiments, the genome includes single or double-stranded DNA or single or double-stranded RNA. In embodiments, the genome includes single-stranded DNA. In embodiments, the genome includes double-stranded DNA. In embodiments, the genome includes single-stranded RNA. In embodiments, the genome includes double-stranded RNA. In embodiments, the virus is a recombinant virus.

### Nucleic Acids

Provided herein, inter alia, are nucleic acids including the promoter provided herein and two or more heterologous nucleic acid sequences. In an aspect is provided a nucleic acid including: i) a promoter having at least 80% sequence identity to the sequence of SEQ ID NO:10 or SEQ ID NO:21; ii) a first heterologous nucleic acid sequence attached to the 3′ end of the promoter; and iii) a second heterologous nucleic acid sequence attached to the 5′ end of the promoter.

In embodiments, the first heterologous nucleic acid sequence encodes a first therapeutic agent or a first detectable agent and the second heterologous nucleic acid sequence is the reverse complement of a sequence encoding a second therapeutic agent or a second detectable agent, or the first heterologous nucleic acid sequence encodes the second therapeutic agent or the second detectable agent and the second heterologous nucleic acid sequence is the reverse complement of a sequence encoding the first therapeutic agent or the first detectable agent. In embodiments, the first heterologous nucleic acid sequence encodes the first therapeutic agent or the first detectable agent and the second heterologous nucleic acid sequence is the reverse complement of a sequence encoding the second therapeutic agent or the second detectable agent. In embodiments, the first heterologous nucleic acid sequence encodes the second therapeutic agent or the second detectable agent and the second heterologous nucleic acid sequence is the reverse complement of a sequence encoding the first therapeutic agent or the first detectable agent.

In embodiments, the first heterologous nucleic acid sequence encodes a first therapeutic agent and the second heterologous nucleic acid sequence is the reverse complement of a sequence encoding a second therapeutic agent, or the first heterologous nucleic acid sequence encodes the second therapeutic agent and the second heterologous nucleic acid sequence is the reverse complement of a sequence encoding the first therapeutic agent. In embodiments, the first heterologous nucleic acid sequence encodes a first therapeutic agent and the second heterologous nucleic acid sequence is the reverse complement of a sequence encoding a second therapeutic agent. In embodiments, the first heterologous nucleic acid sequence encodes the second therapeutic agent and the second heterologous nucleic acid sequence is the reverse complement of a sequence encoding the first therapeutic agent.

In embodiments, the first heterologous nucleic acid sequence encodes a first detectable agent and the second heterologous nucleic acid sequence is the reverse complement of a sequence encoding a second detectable agent, or the first heterologous nucleic acid sequence encodes the second detectable agent and the second heterologous nucleic acid sequence is the reverse complement of a sequence encoding the first detectable agent. In embodiments, the first heterologous nucleic acid sequence encodes a first detectable agent and the second heterologous nucleic acid sequence is the reverse complement of a sequence encoding a second detectable agent. In embodiments, the first heterologous nucleic acid sequence encodes the second detectable agent and the second heterologous nucleic acid sequence is the reverse complement of a sequence encoding the first detectable agent.

In embodiments, the first heterologous nucleic acid sequence encodes a therapeutic agent or a detectable agent. In embodiments, the first heterologous nucleic acid sequence encodes a therapeutic agent. In embodiments, the first heterologous nucleic acid sequence encodes a detectable agent. In embodiments, the second heterologous nucleic acid sequence is the reverse complement of a sequence encoding a therapeutic agent or detectable agent. In embodiments, the second heterologous nucleic acid sequence is the reverse complement of a sequence encoding a therapeutic agent. In embodiments, the second heterologous nucleic acid sequence is the reverse complement of a sequence encoding a detectable agent.

In embodiments, the first heterologous nucleic acid sequence encodes a first therapeutic agent and the second heterologous nucleic acid sequence is the reverse complement of a sequence encoding a second therapeutic agent, or the first heterologous nucleic acid sequence encodes the second therapeutic agent and the second heterologous nucleic acid sequence is the reverse complement of a sequence encoding the first therapeutic agent. In embodiments, the first heterologous nucleic acid sequence encodes a first therapeutic agent and the second heterologous nucleic acid sequence is the reverse complement of a sequence encoding a second therapeutic agent. In embodiments, first heterologous nucleic acid sequence encodes the second therapeutic agent and the second heterologous nucleic acid sequence is the reverse complement of a sequence encoding the first therapeutic agent.

In embodiments, the first therapeutic agent and the second therapeutic agent are independently an RNA-guided DNA endonuclease, an RNA-guided RNA nuclease, a nuclease-deficient RNA-guided DNA endonuclease, a guide RNA (gRNA), a therapeutic protein, a detectable protein, or an antibody. In embodiments, the first therapeutic agent is an RNA-guided DNA endonuclease, an RNA-guided RNA nuclease, a nuclease-deficient RNA-guided DNA endonuclease, a guide RNA (gRNA), a therapeutic protein, a detectable protein, or an antibody. In embodiments, the first therapeutic agent is an RNA-guided DNA endonuclease. In embodiments, the first therapeutic agent is an RNA-guided RNA nuclease. In embodiments, the first therapeutic agent is a nuclease-deficient RNA-guided DNA endonuclease. In embodiments, the first therapeutic agent is a guide RNA (gRNA). In embodiments, the first therapeutic agent is a therapeutic protein. In embodiments, the first therapeutic agent is a detectable protein. In embodiments, the first therapeutic agent is an antibody. In embodiments, the second therapeutic agent is an RNA-guided DNA endonuclease, an RNA-guided RNA nuclease, a nuclease-deficient RNA-guided DNA endonuclease, a guide RNA (gRNA), a therapeutic protein, a detectable protein, or an antibody. In embodiments, the second therapeutic agent is an RNA-guided DNA endonuclease, an RNA-guided RNA nuclease, a nuclease-deficient RNA-guided DNA endonuclease, a guide RNA (gRNA), a therapeutic protein, a detectable protein, or an antibody. In embodiments, the second therapeutic agent is an RNA-guided DNA endonuclease. In embodiments, the second therapeutic agent is an RNA-guided RNA nuclease. In embodiments, the second therapeutic agent is a nuclease-deficient RNA-guided DNA endonuclease. In embodiments, the second therapeutic agent is a guide RNA (gRNA). In embodiments, the second therapeutic agent is a therapeutic protein. In embodiments, the second therapeutic agent is a detectable protein. In embodiments, the second therapeutic agent is an antibody.

In embodiments, the first therapeutic agent an RNA-guided DNA endonuclease and the second therapeutic agent is a gRNA, or the first therapeutic agent is a gRNA and the second therapeutic agent is an RNA-guided DNA endonuclease. In embodiments, the first therapeutic agent an RNA-guided DNA endonuclease and the second therapeutic agent is a gRNA. In embodiments, the first therapeutic agent is a gRNA and the second therapeutic agent is an RNA-guided DNA endonuclease.

In embodiments, the RNA-guided DNA endonuclease is Cas9, Cas12a, a Class II CRISPR endonuclease, or variants thereof. In embodiments, the RNA-guided DNA endonuclease is Cas9 or a variant thereof. In embodiments, the RNA-guided DNA endonuclease is Cas12a or a variant thereof. In embodiments, the RNA-guided DNA endonuclease is a Class II CRISPR endonuclease or a variant thereof.

In embodiments, the first therapeutic agent is a nuclease-deficient RNA-guided DNA endonuclease and the second therapeutic agent is a gRNA, or the first therapeutic agent is the gRNA and the second therapeutic agent is the a nuclease-deficient RNA-guided DNA endonuclease. In embodiments, the first therapeutic agent is a nuclease-deficient RNA-guided DNA endonuclease and the second therapeutic agent is a gRNA. In embodiments, the first therapeutic agent is the gRNA and the second therapeutic agent is the nuclease-deficient RNA-guided DNA endonuclease.

In embodiments, the first therapeutic agent an RNA-guided RNA nuclease and the second therapeutic agent is a gRNA, or the first therapeutic agent is the gRNA and the second therapeutic agent is the RNA-guided RNA nuclease. In embodiments, the first therapeutic agent an RNA-guided RNA nuclease and the second therapeutic agent is a gRNA. In embodiments, the first therapeutic agent is the gRNA and the second therapeutic agent is the RNA-guided RNA nuclease.

In embodiments, the gRNA includes a plurality of gRNAs. For example, the gRNA may include 1, 2, 3, 4, 5 or more gRNAs wherein each gRNA has a different target sequence.

In embodiments, the detectable protein is EGFR, Scarlet, or luciferase. In embodiments, the detectable protein is EGFR. In embodiments, the detectable protein is Scarlet. In embodiments, the detectable protein is luciferase.

For the nucleic acid provided herein, in embodiments, the promoter is from about 150 to about 300 nucleotides in length. In embodiments, the promoter is from about 160 to about 300 nucleotides in length. In embodiments, the promoter is from about 170 to about 300 nucleotides in length. In embodiments, the promoter is from about 180 to about 300 nucleotides in length. In embodiments, the promoter is from about 190 to about 300 nucleotides in length. In embodiments, the promoter is from about 200 to about 300 nucleotides in length. In embodiments, the promoter is from about 210 to about 300 nucleotides in length. In embodiments, the promoter is from about 220 to about 300 nucleotides in length. In embodiments, the promoter is from about 230 to about 300 nucleotides in length. In embodiments, the promoter is from about 240 to about 300 nucleotides in length. In embodiments, the promoter is from about 250 to about 300 nucleotides in length. In embodiments, the promoter is from about 260 to about 300 nucleotides in length. In embodiments, the promoter is from about 270 to about 300 nucleotides in length. In embodiments, the promoter is from about 280 to about 300 nucleotides in length. In embodiments, the promoter is from about 290 to about 300 nucleotides in length.

In embodiments, the promoter is from about 150 to about 290 nucleotides in length. In embodiments, the promoter is from about 150 to about 280 nucleotides in length. In embodiments, the promoter is from about 150 to about 270 nucleotides in length. In embodiments, the promoter is from about 150 to about 260 nucleotides in length. In embodiments, the promoter is from about 150 to about 250 nucleotides in length. In embodiments, the promoter is from about 150 to about 240 nucleotides in length. In embodiments, the promoter is from about 150 to about 230 nucleotides in length. In embodiments, the promoter is from about 150 to about 220 nucleotides in length. In embodiments, the promoter is from about 150 to about 210 nucleotides in length. In embodiments, the promoter is from about 150 to about 200 nucleotides in length. In embodiments, the promoter is from about 150 to about 190 nucleotides in length. In embodiments, the promoter is from about 150 to about 180 nucleotides in length. In embodiments, the promoter is from about 150 to about 170 nucleotides in length. In embodiments, the promoter is from about 150 to about 160 nucleotides in length. In embodiments, the promoter is about 150, 160, 170, 180, 190, 200, 210, 220, 230, 240, 250, 260, 270, 280, 290, or 300 nucleotides in length.

For the nucleic acid provided herein, in embodiments, the promoter is from about 200 to about 250 nucleotides in length. In embodiments, the promoter is from about 205 to about 250 nucleotides in length. In embodiments, the promoter is from about 210 to about 250 nucleotides in length. In embodiments, the promoter is from about 215 to about 250 nucleotides in length. In embodiments, the promoter is from about 220 to about 250 nucleotides in length. In embodiments, the promoter is from about 225 to about 250 nucleotides in length. In embodiments, the promoter is from about 230 to about 250 nucleotides in length. In embodiments, the promoter is from about 235 to about 250 nucleotides in length. In embodiments, the promoter is from about 240 to about 250 nucleotides in length. In embodiments, the promoter is from about 245 to about 250 nucleotides in length.

In embodiments, the promoter is from about 200 to about 245 nucleotides in length. In embodiments, the promoter is from about 200 to about 240 nucleotides in length. In embodiments, the promoter is from about 205 to about 235 nucleotides in length. In embodiments, the promoter is from about 205 to about 230 nucleotides in length. In embodiments, the promoter is from about 205 to about 225 nucleotides in length. In embodiments, the promoter is from about 205 to about 220 nucleotides in length. In embodiments, the promoter is from about 205 to about 215 nucleotides in length. In embodiments, the promoter is from about 205 to about 210 nucleotides in length. In embodiments, the promoter is about 205, 210, 215, 220, 225, 230, 235, 240, 245, or 250 nucleotides in length. In embodiments, the promoter is about 205 nucleotides in length. In embodiments, the promoter is 205 nucleotides in length. In embodiments, the promoter is about 235 nucleotides in length. In embodiments, the promoter is 235 nucleotides in length.

In embodiments, the promoter has at least 80%, 85%, 90%, 95%, 96%, 97%, 98%, 99% or 100% sequence identity across the whole sequence or a portion of the sequence (e.g., a portion comprising at least 20, 30, or 40 continuous nucleotides) compared to SEQ ID NO: 10. In embodiments, the promoter has at least 50%, 80%, 90%, 95%, 96%, 97%, 98%, 99% or 100% activity compared to SEQ ID NO: 10. In embodiments, the promoter has at least 80% sequence identity to the sequence of SEQ ID NO: 10. In embodiments, the promoter has at least 81% sequence identity to the sequence of SEQ ID NO: 10. In embodiments, the promoter has at least 82% sequence identity to the sequence of SEQ ID NO: 10. In embodiments, the promoter has at least 83% sequence identity to the sequence of SEQ ID NO:10. In embodiments, the promoter has at least 84% sequence identity to the sequence of SEQ ID NO: 10. In embodiments, the promoter has at least 85% sequence identity to the sequence of SEQ ID NO: 10. In embodiments, the promoter has at least 86% sequence identity to the sequence of SEQ ID NO:10. In embodiments, the promoter has at least 87% sequence identity to the sequence of SEQ ID NO: 10. In embodiments, the promoter has at least 88% sequence identity to the sequence of SEQ ID NO: 10. In embodiments, the promoter has at least 89% sequence identity to the sequence of SEQ ID NO:10. In embodiments, the promoter has at least 90% sequence identity to the sequence of SEQ ID NO:10. In embodiments, the promoter has at least 91% sequence identity to the sequence of SEQ ID NO: 10. In embodiments, the promoter has at least 92% sequence identity to the sequence of SEQ ID NO: 10. In embodiments, the promoter has at least 93% sequence identity to the sequence of SEQ ID NO: 10. In embodiments, the promoter has at least 94% sequence identity to the sequence of SEQ ID NO: 10. In embodiments, the promoter has at least 95% sequence identity to the sequence of SEQ ID NO: 10. In embodiments, the promoter has at least 96% sequence identity to the sequence of SEQ ID NO: 10. In embodiments, the promoter has at least 97% sequence identity to the sequence of SEQ ID NO:10. In embodiments, the promoter has at least 98% sequence identity to the sequence of SEQ ID NO: 10. In embodiments, the promoter has at least 99% sequence identity to the sequence of SEQ ID NO: 10.

In embodiments, the promoter has about 80% sequence identity to the sequence of SEQ ID NO: 10. In embodiments, the promoter has about 81% sequence identity to the sequence of SEQ ID NO:10. In embodiments, the promoter has about 82% sequence identity to the sequence of SEQ ID NO:10. In embodiments, the promoter has about 83% sequence identity to the sequence of SEQ ID NO:10. In embodiments, the promoter has about 84% sequence identity to the sequence of SEQ ID NO: 10. In embodiments, the promoter has about 85% sequence identity to the sequence of SEQ ID NO: 10. In embodiments, the promoter has about 86% sequence identity to the sequence of SEQ ID NO: 10. In embodiments, the promoter has about 87% sequence identity to the sequence of SEQ ID NO:10. In embodiments, the promoter has about 88% sequence identity to the sequence of SEQ ID NO:10. In embodiments, the promoter has about 89% sequence identity to the sequence of SEQ ID NO:10. In embodiments, the promoter has about 90% sequence identity to the sequence of SEQ ID NO: 10. In embodiments, the promoter has about 91% sequence identity to the sequence of SEQ ID NO: 10. In embodiments, the promoter has about 92% sequence identity to the sequence of SEQ ID NO: 10. In embodiments, the promoter has about 93% sequence identity to the sequence of SEQ ID NO:10. In embodiments, the promoter has about 94% sequence identity to the sequence of SEQ ID NO: 10. In embodiments, the promoter has about 95% sequence identity to the sequence of SEQ ID NO: 10. In embodiments, the promoter has about 96% sequence identity to the sequence of SEQ ID NO:10. In embodiments, the promoter has about 97% sequence identity to the sequence of SEQ ID NO:10. In embodiments, the promoter has about 98% sequence identity to the sequence of SEQ ID NO:10. In embodiments, the promoter has about 99% sequence identity to the sequence of SEQ ID NO: 10. In embodiments, the promoter includes the sequence of SEQ ID NO: 10. In embodiments, the promoter is the sequence of SEQ ID NO:10.

In embodiments, the promoter has at least 80% sequence identity, at least 85%, at least 90%, at least 95% sequence identity, or at least 98% sequence identity to SEQ ID NO:10, wherein the sequence is not SEQ ID NO: 10. In embodiments, the promoter does not include the sequence of SEQ ID NO: 10.

In embodiments, the promoter has at least 80%, 85%, 90%, 95%, 96%, 97%, 98%, 99% or 100% sequence identity across the whole sequence or a portion of the sequence (e.g., a portion comprising at least 20, 30, or 40 continuous nucleotides) compared to SEQ ID NO:21. In embodiments, the promoter has at least 50%, 80%, 90%, 95%, 96%, 97%, 98%, 99% or 100% activity compared to SEQ ID NO:21. Activity can be assessed, for example, by measuring expression levels of the heterologous nucleic acid sequence operably linked to the promoter, for example by qPCR, Western Blot, etc. or any other method known in the biological arts. Expression levels may be measured by detecting the function or activity of a therapeutic or detectable agent encoded by the heterologous nucleic acid sequence operably linked to the promoter. In embodiments, the promoter has at least 80% sequence identity to the sequence of SEQ ID NO:21. In embodiments, the promoter has at least 81% sequence identity to the sequence of SEQ ID NO:21. In embodiments, the promoter has at least 82% sequence identity to the sequence of SEQ ID NO:21. In embodiments, the promoter has at least 83% sequence identity to the sequence of SEQ ID NO:21. In embodiments, the promoter has at least 84% sequence identity to the sequence of SEQ ID NO:21. In embodiments, the promoter has at least 85% sequence identity to the sequence of SEQ ID NO:21. In embodiments, the promoter has at least 86% sequence identity to the sequence of SEQ ID NO:21. In embodiments, the promoter has at least 87% sequence identity to the sequence of SEQ ID NO:21. In embodiments, the promoter has at least 88% sequence identity to the sequence of SEQ ID NO:21. In embodiments, the promoter has at least 89% sequence identity to the sequence of SEQ ID NO:21. In embodiments, the promoter has at least 90% sequence identity to the sequence of SEQ ID NO: 21. In embodiments, the promoter has at least 91% sequence identity to the sequence of SEQ ID NO: 21. In embodiments, the promoter has at least 92% sequence identity to the sequence of SEQ ID NO:21. In embodiments, the promoter has at least 93% sequence identity to the sequence of SEQ ID NO:21. In embodiments, the promoter has at least 94% sequence identity to the sequence of SEQ ID NO:21. In embodiments, the promoter has at least 95% sequence identity to the sequence of SEQ ID NO:21. In embodiments, the promoter has at least 96% sequence identity to the sequence of SEQ ID NO:21. In embodiments, the promoter has at least 97% sequence identity to the sequence of SEQ ID NO:21. In embodiments, the promoter has at least 98% sequence identity to the sequence of SEQ ID NO:21. In embodiments, the promoter has at least 99% sequence identity to the sequence of SEQ ID NO:21.

In embodiments, the promoter has about 80% sequence identity to the sequence of SEQ ID NO: 21. In embodiments, the promoter has about 81% sequence identity to the sequence of SEQ ID NO:21. In embodiments, the promoter has about 82% sequence identity to the sequence of SEQ ID NO:21. In embodiments, the promoter has about 83% sequence identity to the sequence of SEQ ID NO:21. In embodiments, the promoter has about 84% sequence identity to the sequence of SEQ ID NO:21. In embodiments, the promoter has about 85% sequence identity to the sequence of SEQ ID NO:21. In embodiments, the promoter has about 86% sequence identity to the sequence of SEQ ID NO:21. In embodiments, the promoter has about 87% sequence identity to the sequence of SEQ ID NO:21. In embodiments, the promoter has about 88% sequence identity to the sequence of SEQ ID NO:21. In embodiments, the promoter has about 89% sequence identity to the sequence of SEQ ID NO:21. In embodiments, the promoter has about 90% sequence identity to the sequence of SEQ ID NO:21. In embodiments, the promoter has about 91% sequence identity to the sequence of SEQ ID NO:21. In embodiments, the promoter has about 92% sequence identity to the sequence of SEQ ID NO:21. In embodiments, the promoter has about 93% sequence identity to the sequence of SEQ ID NO:21. In embodiments, the promoter has about 94% sequence identity to the sequence of SEQ ID NO: 21. In embodiments, the promoter has about 95% sequence identity to the sequence of SEQ ID NO: 21. In embodiments, the promoter has about 96% sequence identity to the sequence of SEQ ID NO:21. In embodiments, the promoter has about 97% sequence identity to the sequence of SEQ ID NO:21. In embodiments, the promoter has about 98% sequence identity to the sequence of SEQ ID NO:21. In embodiments, the promoter has about 99% sequence identity to the sequence of SEQ ID NO:21. In embodiments, the promoter includes the sequence of SEQ ID NO: 21. In embodiments, the promoter is the sequence of SEQ ID NO:21.

In embodiments, the nucleic acid further comprises one or more introns. In embodiments, the intron links the promoter to the first heterologous nucleic acid sequence, or links the promoter to the second heterologous nucleic acid sequence. In embodiments, the intron links the promoter to the first heterologous nucleic acid sequence. In embodiments, the intron links the promoter to the second heterologous nucleic acid sequence.

In embodiments, the intron has at least 80%, 85%, 90%, 95%, 96%, 97%, 98%, 99% or 100% sequence identity across the whole sequence or a portion of the sequence (e.g., a portion comprising at least 20, 30, or 40 continuous nucleotides) compared to SEQ ID NO:22. In embodiments, the intron includes a sequence having at least 80% sequence identity to the SEQ ID NO: 22. In embodiments, the intron includes a sequence having at least 81% sequence identity to the SEQ ID NO:22. In embodiments, the intron includes a sequence having at least 82% sequence identity to the SEQ ID NO:22. In embodiments, the intron includes a sequence having at least 83% sequence identity to the SEQ ID NO:22. In embodiments, the intron includes a sequence having at least 84% sequence identity to the SEQ ID NO:22. In embodiments, the intron includes a sequence having at least 85% sequence identity to the SEQ ID NO:22. In embodiments, the intron includes a sequence having at least 86% sequence identity to the SEQ ID NO: 22. In embodiments, the intron includes a sequence having at least 87% sequence identity to the SEQ ID NO:22. In embodiments, the intron includes a sequence having at least 88% sequence identity to the SEQ ID NO:22. In embodiments, the intron includes a sequence having at least 89% sequence identity to the SEQ ID NO:22. In embodiments, the intron includes a sequence having at least 90% sequence identity to the SEQ ID NO:22. In embodiments, the intron includes a sequence having at least 91% sequence identity to the SEQ ID NO:22. In embodiments, the intron includes a sequence having at least 92% sequence identity to the SEQ ID NO: 22. In embodiments, the intron includes a sequence having at least 93% sequence identity to the SEQ ID NO:22. In embodiments, the intron includes a sequence having at least 94% sequence identity to the SEQ ID NO:22. In embodiments, the intron includes a sequence having at least 95% sequence identity to the SEQ ID NO:22. In embodiments, the intron includes a sequence having at least 96% sequence identity to the SEQ ID NO:22. In embodiments, the intron includes a sequence having at least 97% sequence identity to the SEQ ID NO:22. In embodiments, the intron includes a sequence having at least 98% sequence identity to the SEQ ID NO: 22. In embodiments, the intron includes a sequence having at least 99% sequence identity to the SEQ ID NO:22.

In embodiments, the intron includes a sequence having about 80% sequence identity to the SEQ ID NO:22. In embodiments, the intron includes a sequence having about 81% sequence identity to the SEQ ID NO:22. In embodiments, the intron includes a sequence having about 82% sequence identity to the SEQ ID NO:22. In embodiments, the intron includes a sequence having about 83% sequence identity to the SEQ ID NO:22. In embodiments, the intron includes a sequence having about 84% sequence identity to the SEQ ID NO:22. In embodiments, the intron includes a sequence having about 85% sequence identity to the SEQ ID NO:22. In embodiments, the intron includes a sequence having about 86% sequence identity to the SEQ ID NO: 22. In embodiments, the intron includes a sequence having about 87% sequence identity to the SEQ ID NO:22. In embodiments, the intron includes a sequence having about 88% sequence identity to the SEQ ID NO:22. In embodiments, the intron includes a sequence having about 89% sequence identity to the SEQ ID NO:22. In embodiments, the intron includes a sequence having about 90% sequence identity to the SEQ ID NO:22. In embodiments, the intron includes a sequence having about 91% sequence identity to the SEQ ID NO:22. In embodiments, the intron includes a sequence having about 92% sequence identity to the SEQ ID NO:22. In embodiments, the intron includes a sequence having about 93% sequence identity to the SEQ ID NO: 22. In embodiments, the intron includes a sequence having about 94% sequence identity to the SEQ ID NO:22. In embodiments, the intron includes a sequence having about 95% sequence identity to the SEQ ID NO:22. In embodiments, the intron includes a sequence having about 96% sequence identity to the SEQ ID NO:22. In embodiments, the intron includes a sequence having about 97% sequence identity to the SEQ ID NO:22. In embodiments, the intron includes a sequence having about 98% sequence identity to the SEQ ID NO:22. In embodiments, the intron includes a sequence having about 99% sequence identity to the SEQ ID NO:22. In embodiments, the intron includes the sequence of SEQ ID NO:22. In embodiments, the intron is the sequence of SEQ ID NO:22.

In embodiments, the intron has at least 80%, 85%, 90%, 95%, 96%, 97%, 98%, 99% or 100% sequence identity across the whole sequence or a portion of the sequence (e.g., a portion comprising at least 20, 30, or 40 continuous nucleotides) compared to SEQ ID NO:23. In embodiments, the intron includes a sequence having at least 80% sequence identity to the SEQ ID NO: 23. In embodiments, the intron includes a sequence having at least 81% sequence identity to the SEQ ID NO:23. In embodiments, the intron includes a sequence having at least 82% sequence identity to the SEQ ID NO:23. In embodiments, the intron includes a sequence having at least 83% sequence identity to the SEQ ID NO:23. In embodiments, the intron includes a sequence having at least 84% sequence identity to the SEQ ID NO:23. In embodiments, the intron includes a sequence having at least 85% sequence identity to the SEQ ID NO:23. In embodiments, the intron includes a sequence having at least 86% sequence identity to the SEQ ID NO: 23. In embodiments, the intron includes a sequence having at least 87% sequence identity to the SEQ ID NO:23. In embodiments, the intron includes a sequence having at least 88% sequence identity to the SEQ ID NO:23. In embodiments, the intron includes a sequence having at least 89% sequence identity to the SEQ ID NO:23. In embodiments, the intron includes a sequence having at least 90% sequence identity to the SEQ ID NO:23. In embodiments, the intron includes a sequence having at least 91% sequence identity to the SEQ ID NO:23. In embodiments, the intron includes a sequence having at least 92% sequence identity to the SEQ ID NO: 23. In embodiments, the intron includes a sequence having at least 93% sequence identity to the SEQ ID NO:23. In embodiments, the intron includes a sequence having at least 94% sequence identity to the SEQ ID NO:23. In embodiments, the intron includes a sequence having at least 95% sequence identity to the SEQ ID NO:23. In embodiments, the intron includes a sequence having at least 96% sequence identity to the SEQ ID NO:23. In embodiments, the intron includes a sequence having at least 97% sequence identity to the SEQ ID NO:23. In embodiments, the intron includes a sequence having at least 98% sequence identity to the SEQ ID NO: 23. In embodiments, the intron includes a sequence having at least 99% sequence identity to the SEQ ID NO:23.

In embodiments, the intron includes a sequence having about 80% sequence identity to the SEQ ID NO:23. In embodiments, the intron includes a sequence having about 81% sequence identity to the SEQ ID NO:23. In embodiments, the intron includes a sequence having about 82% sequence identity to the SEQ ID NO:23. In embodiments, the intron includes a sequence having about 83% sequence identity to the SEQ ID NO:23. In embodiments, the intron includes a sequence having about 84% sequence identity to the SEQ ID NO:23. In embodiments, the intron includes a sequence having about 85% sequence identity to the SEQ ID NO:23. In embodiments, the intron includes a sequence having about 86% sequence identity to the SEQ ID NO: 23. In embodiments, the intron includes a sequence having about 87% sequence identity to the SEQ ID NO:23. In embodiments, the intron includes a sequence having about 88% sequence identity to the SEQ ID NO:23. In embodiments, the intron includes a sequence having about 89% sequence identity to the SEQ ID NO:23. In embodiments, the intron includes a sequence having about 90% sequence identity to the SEQ ID NO:23. In embodiments, the intron includes a sequence having about 91% sequence identity to the SEQ ID NO:23. In embodiments, the intron includes a sequence having about 92% sequence identity to the SEQ ID NO:23. In embodiments, the intron includes a sequence having about 93% sequence identity to the SEQ ID NO: 23. In embodiments, the intron includes a sequence having about 94% sequence identity to the SEQ ID NO:23. In embodiments, the intron includes a sequence having about 95% sequence identity to the SEQ ID NO:23. In embodiments, the intron includes a sequence having about 96% sequence identity to the SEQ ID NO:23. In embodiments, the intron includes a sequence having about 97% sequence identity to the SEQ ID NO:23. In embodiments, the intron includes a sequence having about 98% sequence identity to the SEQ ID NO:23. In embodiments, the intron includes a sequence having about 99% sequence identity to the SEQ ID NO:23. In embodiments, the intron includes the sequence of SEQ ID NO:23. In embodiments, the intron is the sequence of SEQ ID NO:23.

In embodiments, the insulator sequence is attached to the 5′ end or 3′ end of the first heterologous nucleic acid sequence or the second heterologous nucleic acid sequence. In embodiments, the insulator sequence is attached to the 5′ end of the first heterologous nucleic acid sequence. In embodiments, the insulator sequence is attached to the 5′ end of the second heterologous nucleic acid sequence. In embodiments, the insulator sequence is attached to 3′ end of the first heterologous nucleic acid sequence. In embodiments, the insulator sequence is attached to the 3′ end of the second heterologous nucleic acid sequence.

In embodiments, the insulator sequence includes the sequence of SEQ ID NO:24. In embodiments, the insulator sequence is the sequence of SEQ ID NO:24.

For the nucleic acid provided herein, in embodiments, the nucleic acid is single or double-stranded DNA or RNA. In embodiments, the nucleic acid is single-stranded DNA. In embodiments, the nucleic acid is double-stranded DNA. In embodiments, the nucleic acid is RNA.

In an aspect is provided a vector, such as an expression vector, that has any of the nucleotide sequences of the present disclosure. Therefore, in embodiments, the expression vector can be used to produce the therapeutic and/or detectable agent encoded by the first and/or second heterologous nucleic acid sequence provided herein. The expression vector can be packaged into a virus or transfected into cells (e.g. eukaryotic cells such as mammalian cells or human cell lines or prokaryotic cells such as Escherichia coli). Expression vectors contemplated include, but are not limited to, viral vectors (e.g. AAV vector) based on various viral sequences as well as those contemplated for eukaryotic target cells or prokaryotic target cells. The “target cells” (e.g. retinal ganglion cells) may refer to the cells where the expression vector is transfected and the heterologous nucleic acid sequence encoding the therapeutic or detectable agent is expressed. Thus, in an aspect is provided an expression vector including the nucleic acid provided herein including embodiments thereof.

In embodiments, the expression vector is an adeno-associated viral (AAV) vector. In embodiments, the AAV vector may selected from AAV1, AAV2, AAV3b, AAV4, AAV5, AAV6, AAV7, AAV8, AAV9, AAV10, AAV-11, AAV-12, AAV113, AAVrh.74, AAV2.7m8, AAV.ANC80, Anc80L65 and derivatives thereof, and AAVDJ, and combinations thereof. An extensive listing of illustrative AAV serotypes, including variants of naturally occurring AAV serotypes, is provided in U.S. Pat. No. 10,662,425, which is incorporated herein in its entirety and for all purposes.

In embodiments, the expression vector includes sequences including those of AAV that are required in cis for replication and packaging (e.g., functional ITRs) of the DNA into a virion. In embodiments, AAV vectors have one or more of the AAV WT genes deleted in whole or in part, but retain functional flanking ITR sequences.

### Methods of Use

The compositions provided herein (e.g. viruses and nucleic acids), are contemplated to be useful for expressing one or more therapeutic and/or detectable agents in a cell. The compositions include promoters capable of functioning as bidirectional promoters for gene expression in a cell (e.g. a neuron). Thus, in an aspect is provided a method of expressing a first heterologous nucleic acid sequence and a second heterologous nucleic acid sequence, including contacting a cell with a virus provided herein including embodiments thereof, and allowing the cell to express the first heterologous nucleic acid sequence and the second heterologous nucleic acid sequence.

In embodiments, the cell is a mammalian cell. In embodiments, the cell is a mammalian neuronal cell. In some embodiments, the neuronal cell is a sensory neuron. In embodiments, the cell is a retinal ganglion cell (RGC), a photoreceptor cell, or a retinal pigmented epithelial (RPE) cell. In some embodiments, the cell is an ophthalmic neuron such as a bipolar cell, an amacrine cell, or a horizontal cell. In embodiments, the cell is a retinal ganglion cell (RGC). In embodiments, the cell is a photoreceptor cell. In embodiments, the cell is an RPE cell.

In another aspect is provided a method of expressing a first heterologous nucleic acid sequence and a second heterologous nucleic acid sequence, comprising contacting a cell with a nucleic acid provided herein including embodiments thereof, or the expression vector provided herein including embodiments thereof, and allowing the cell to express the first heterologous nucleic acid sequence and the second heterologous nucleic acid sequence.

In embodiments, the cell is a mammalian cell. In embodiments, the cell is a mammalian neuronal cell. In embodiments, the cell is a retinal ganglion cell (RGC), a photoreceptor cell, or a retinal pigmented epithelial (RPE) cell. In embodiments, the cell is a retinal ganglion cell (RGC). In embodiments, the cell is a photoreceptor cell. In embodiments, the cell is an RPE cell.

The compositions provided herein are contemplated to be effective for treatment of diseases (e.g. glaucoma) typically characterized by suboptimal neuron survival and/or function. The compositions are surprisingly effective in delivering heterologous nucleic acids encoding therapeutic agents to neuron cells, thereby allowing treatment of the diseases. Thus, in another aspect is provided a method of treating a disease in a subject in need thereof, the method including administering to said subject an effective amount of a virus provided herein including embodiments thereof, a nucleic acid provided herein including embodiments thereof, or an expression vector provided herein including embodiments thereof.

In embodiments, the method includes administering an effective amount of a virus provided herein including embodiments thereof. In embodiments, the method includes administering an effective amount of a nucleic acid provided herein including embodiments thereof. In embodiments, the method includes administering an effective amount of an expression vector provided herein including embodiments thereof.

In embodiments, the disease is a neurodegenerative disease. In embodiments, the disease is a neurodegenerative disease of the eye. In embodiments, the disease is glaucoma, age-related macular degeneration (AMD), or choroidal neovascularization (CNV), myopia-associated CNV, diabetic retinopathy, macular oedema, and retinal vein occlusion, or retinal pigmentosa. In embodiments, the disease is glaucoma. In embodiments, the disease is AMD. In embodiments, the disease is CNV. In embodiments, the disease is myopia-associated CNV. In embodiments, the disease is diabetic retinopathy. In embodiments, the disease is macular oedema. In embodiments, the disease is retinal vein occlusion. In embodiments, the disease is retinal pigmentosa.

For the methods provided herein, in embodiments, a vector (e.g. an expression vector (e.g. an AAV vector)) comprising a promoter (e.g. promoter 10 or a variant thereof, etc.) that is capable of expressing a first and a second heterologous nucleic acid sequence from either side of the promoter is administered in an “effective amount” or a “therapeutically effective amount,” i.e., an amount that is effective, at dosages and for periods of time necessary, to achieve a desired result. For example, the desired effect upon expression of the first and the second therapeutic agent (e.g. neuroprotective agent) in cells (e.g. ophthalmic neurons, RGC cells, etc.) may include a detectable improvement in a symptom associated with axon or neuron protection; or associated with a symptom of a disease (e.g. glaucoma). Alternatively, if the vector provided herein is used prophylactically, a desired result would include a demonstrable prevention of one or more symptoms of a disease (e.g. glaucoma).

For the methods provided herein, in embodiments, the vector (e.g. expression vector) is a viral vector, such as an AAV vector, administered in a pharmaceutically acceptable vehicle, excipient or diluent. A “pharmaceutically acceptable” is a carrier, diluent, excipient, and/or salt that is compatible with the other ingredients of the formulation, and not deleterious to the recipient thereof. Examples include phosphate buffered saline with poloxamer, phosphate buffered saline, or saline. The therapeutic agent may be provided in various forms such as a suspension or solution. The therapeutic agent may be formulated for administration along with formulatory agents such as suspending, stabilizing and/or dispersing agents, preservatives, and buffers. Alternatively, the therapeutic agent may be provided as a lyophilizate or aseptic isolation of sterile solid, for constitution with a suitable vehicle, e.g., sterile, pyrogen-free water, before use. When the therapeutic agents of the invention are prepared for administration, they may be combined with a pharmaceutically acceptable carrier, diluent or excipient to form a pharmaceutical formulation, or unit dosage form.

In embodiments, administration is administration to the eye, for example, by intravitreal injection. In embodiments, the composition (e.g. nucleic acid, expression vector, AAV viral vector, etc.) may be administered in a volume of 5 to 500 microliters, often 100 to 500 microliters, more often 100 to 400 microliters, and most often 100 to 250 microliters, e.g. 100 microliters, 150 microliters, 200 microliters, or 250 microliters. In embodiments, the composition is administered in a volume of 5 microliters, 100 microliters, 150 microliters, 200 microliters, 250 microliters, 400 microliters or 500 microliters.

In embodiments, administration of a viral vector (e.g. an expression vector, aAAV viral vector, etc.) to a human includes administration of a dosage of 10⁹ to 10¹³ viral vectors per injection. In embodiments, 10⁹ to 10¹⁰, 10¹⁰ to 10¹¹, 10¹¹ to 10¹², or 10¹² to 10¹³ viral vectors are administered per injection. In embodiments, 10⁹, 10¹⁰, 10¹⁰, 10¹¹, 10¹¹, 10¹², or 10¹³ viral vectors are administered per injection.

In embodiments, the vector (e.g. expression vector, viral vector) provided herein is administered in a virus (e.g. virion, virus particle). In embodiments, the vector (e.g. expression vector) is administered as a “naked” DNA or RNA, i.e. not packaged in a virus (e.g. virion, virus particle).

Dosage regimens may be adjusted to provide the optimum therapeutic response. For example, a single bolus may be administered, several divided doses may be administered over time or the dose may be proportionally reduced or increased as indicated by the exigencies of the therapeutic situation.

Multiple treatments, including in combination with other gene therapy vectors, may be administered to any subject, including over the lifetime of the subject.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Cell Compositions

The compositions provided herein are contemplated to be effective for delivering and expressing heterologous nucleic acids in a cell. Thus, in an aspect is provided a cell including a virus provided herein including embodiments thereof, a nucleic acid provided herein including embodiments thereof, or an expression vector provided herein including embodiments thereof. In embodiments, the cell includes a virus provided herein including embodiments thereof. In embodiments, the cell includes a nucleic acid provided herein including embodiments thereof. In embodiments, the cell includes an expression vector provided herein including embodiments thereof.

In embodiments, the cell is a mammalian cell. In some embodiments, the cell is a mammalian neuronal cell. In some embodiments, the cell is a sensory neuron. In some embodiments, the cell is a retinal ganglion cell (RGC), a photoreceptor cell, or a pigmented epithelial cell. In some embodiments, the cell is an ophthalmic neuron such as a bipolar cell, an amacrine cell, or a horizontal cell.

### P Embodiments

Embodiment P1: A bidirectional promoter to drive expression of two mRNAs in gene therapy.

Embodiment P2: The bidirectional promoter of embodiment P1, wherein the bidirectional promoter is Promoter 10.

Embodiment P3: The bidirectional promoter of embodiment P1, wherein the gene therapy is AAV-based gene therapy.

Embodiment P4: The bidirectional promoter of embodiment P1, wherein the promoter enables the delivery of CRISPR machinery to neurons.

Embodiment P5: A method of expression two mRNAs in gene therapy comprising constructing and delivering a construct comprising the bidirectional promoter of embodiment P1 in a gene therapy vector.

Embodiment P6: The method of embodiment P5, wherein the bidirectional promoter is Promoter 10.

Embodiment P7: The method of embodiment P5, wherein the gene therapy is AAV-based gene therapy.

Embodiment P8: The method of embodiment P5, wherein the promoter enables the delivery of CRISPR machinery to neurons.

### Embodiments

Embodiment 1: A virus having a genome comprising: i) a promoter having at least 80% sequence identity to the sequence of SEQ ID NO: 10 or SEQ ID NO:21; ii) a first heterologous nucleic acid sequence attached to the 3′ end of the promoter; and iii) a second heterologous nucleic acid sequence attached to the 5′ end of the promoter.

Embodiment 2: The virus of embodiment 1, wherein: the first heterologous nucleic acid sequence encodes a first therapeutic agent or a first detectable agent and the second heterologous nucleic acid sequence is the reverse complement of a sequence encoding a second therapeutic agent or a second detectable agent, or the first heterologous nucleic acid sequence encodes the second therapeutic agent or the second detectable agent and the second heterologous nucleic acid sequence is the reverse complement of a sequence encoding the first therapeutic agent or the first detectable agent.

Embodiment 3: The virus of embodiment 2, wherein the first therapeutic agent and the second therapeutic agent are independently an RNA-guided DNA endonuclease, an RNA-guided RNA nuclease, a nuclease-deficient RNA-guided DNA endonuclease, a guide RNA (gRNA), a therapeutic protein, or an antibody.

Embodiment 4: The virus of embodiment 3, wherein the first therapeutic agent is an RNA-guided DNA endonuclease and the second therapeutic agent is a gRNA, or the first therapeutic agent is the gRNA and the second therapeutic agent is the RNA-guided DNA endonuclease.

Embodiment 5: The virus of embodiment 3 or 4, wherein the RNA-guided DNA endonuclease is Cas9, Cas12a, a Class II CRISPR endonuclease or variants thereof.

Embodiment 6: The virus of any one of embodiments 3-5, wherein the gRNA comprises a plurality of gRNAs.

Embodiment 7: The virus of embodiment 2, wherein the first detectable agent and the second detectable agent are independently a detectable protein.

Embodiment 8: The virus of embodiment 7, wherein the detectable protein is EGFR, Scarlet, or luciferase.

Embodiment 9: The virus of any one of embodiments 1-8, wherein the promoter is from about 150 to about 300 nucleotides in length.

Embodiment 10: The virus of embodiment 9, wherein the promoter is from about 200 to about 250 nucleotides in length.

Embodiment 11: The virus of any one of embodiments 1-10, wherein the promoter comprises the sequence of SEQ ID NO: 10 or SEQ ID NO:21.

Embodiment 12: The virus of embodiment 11, wherein the promoter comprises the sequence of SEQ ID NO:10.

Embodiment 13: The virus of embodiment 11, wherein the promoter comprises the sequence of SEQ ID NO:21.

Embodiment 14: The virus of any one of embodiments 1-13, wherein the genome further comprises one or more introns.

Embodiment 15: The virus of embodiment 14, wherein the intron links the promoter to the first heterologous nucleic acid sequence, or links the promoter to the second heterologous nucleic acid sequence.

Embodiment 16: The virus of embodiment 14 or 15, wherein the intron comprises a sequence having at least 80% sequence identity to the SEQ ID NO:22 or SEQ ID NO:23.

Embodiment 17: The virus of embodiment 16, wherein the intron comprises the sequence of SEQ ID NO:22 or SEQ ID NO:23.

Embodiment 18: The virus of any one of embodiments 1-17, wherein the genome further comprises one or more insulator sequences.

Embodiment 19: The virus of embodiment 18, wherein the insulator sequence is attached to the 5′ end or 3′ end of the first heterologous nucleic acid sequence or the second heterologous nucleic acid sequence.

Embodiment 20: The virus of embodiment 18 or 19, wherein the insulator sequence comprises the sequence of SEQ ID NO:24.

Embodiment 21: The virus of any one of embodiments 1-21, wherein said virus is a herpes simplex virus, an adeno-associated virus (AAV), a lentivirus, an adenovirus, a vaccinia virus, a poxvirus, an alphavirus, an enterovirus, a papillomavirus, or a poliovirus.

Embodiment 22: The virus of embodiment 21, wherein said virus is an AAV.

Embodiment 23: The virus of any one of embodiments 1-22, wherein the genome comprises single-stranded DNA or double-stranded DNA.

Embodiment 24: The virus of any one of embodiments 1-21, wherein the genome comprises single-stranded RNA or double-stranded RNA.

Embodiment 25: The virus of any one of embodiments 1-24, wherein the virus is a recombinant virus.

Embodiment 26: A nucleic acid comprising: i) a promoter having at least 80% sequence identity to the sequence of SEQ ID NO: 10 or SEQ ID NO:21; ii) a first heterologous nucleic acid sequence attached to the 3′ end of the promoter; and iii) a second heterologous nucleic acid sequence attached to the 5′ end of the promoter.

Embodiment 27: The nucleic acid of embodiment 26, wherein: the first heterologous nucleic acid sequence encodes a first therapeutic agent or a first detectable agent and the second heterologous nucleic acid sequence is the reverse complement of a sequence encoding a second therapeutic agent or a second detectable agent, or the first heterologous nucleic acid sequence encodes the second therapeutic agent or the second detectable agent and the second heterologous nucleic acid sequence is the reverse complement of a sequence encoding the first therapeutic agent or the first detectable agent.

Embodiment 28: The nucleic acid of embodiment 27, wherein the first therapeutic agent and the second therapeutic agent are independently an RNA-guided DNA endonuclease, an RNA-guided RNA nuclease, a nuclease-deficient RNA-guided DNA endonuclease, a guide RNA (gRNA), a therapeutic protein, or an antibody.

Embodiment 29: The nucleic acid of embodiment 28, wherein the first therapeutic agent is an RNA-guided DNA endonuclease and the second therapeutic agent is a gRNA, or the first therapeutic agent is a gRNA and the second therapeutic agent is an RNA-guided DNA endonuclease.

Embodiment 30: The nucleic acid of embodiment 28 or 29, wherein the RNA-guided DNA endonuclease is Cas9, Cas12a, a Class II CRISPR endonuclease, or variants thereof.

Embodiment 31: The nucleic acid of any one of embodiments 28-30, wherein the gRNA comprises a plurality of gRNAs.

Embodiment 32: The nucleic acid of embodiment 27, wherein the first detectable agent and the second detectable agent are independently a detectable protein.

Embodiment 33: The nucleic acid of embodiment 32, wherein the detectable protein is EGFR, Scarlet, or luciferase.

Embodiment 34: The nucleic acid of any one of embodiments 26-33, wherein the promoter is from about 150 to about 300 nucleotides in length.

Embodiment 35: The nucleic acid of embodiment 34, wherein the promoter is from about 200 to about 250 nucleotides in length.

Embodiment 36: The nucleic acid of any one of embodiments 26-35, wherein the promoter comprises the sequence of SEQ ID NO: 10 or SEQ ID NO:21.

Embodiment 37: The nucleic acid of embodiment 36, wherein the promoter comprises the sequence of SEQ ID NO: 10.

Embodiment 38: The nucleic acid of embodiment 36, wherein the promoter comprises the sequence of SEQ ID NO:21.

Embodiment 39: The nucleic acid of any one of embodiments 26-38, wherein the nucleic acid further comprises one or more introns.

Embodiment 40: The nucleic acid embodiment 39, wherein the intron links the promoter to the first heterologous nucleic acid sequence, or links the promoter to the second heterologous nucleic acid sequence.

Embodiment 41: The nucleic acid embodiment 39 or 40, wherein the intron comprises a sequence having at least 80% sequence identity to the SEQ ID NO:22 or SEQ ID NO:23.

Embodiment 42: The nucleic acid of embodiment 41, wherein the intron comprises the sequence of SEQ ID NO:22 or SEQ ID NO:23.

Embodiment 43: The nucleic acid of any one of embodiments 26-42, wherein the nucleic acid further comprises one or more insulator sequences.

Embodiment 44: The nucleic acid of embodiment 43, wherein the insulator sequence is attached to the 5′ end or 3′ end of the first heterologous nucleic acid sequence or the second heterologous nucleic acid sequence.

Embodiment 45: The nucleic acid of embodiment 43 or 44, wherein the insulator sequence comprises the sequence of SEQ ID NO:24.

Embodiment 46: An expression vector comprising the nucleic acid of any one of embodiments 26-45.

Embodiment 47: A method of expressing a first heterologous nucleic acid sequence and a second heterologous nucleic acid sequence, comprising contacting a cell with the virus of any one of embodiments 1-25, and allowing the cell to express the first heterologous nucleic acid sequence and the second heterologous nucleic acid sequence.

Embodiment 48: The method of embodiment 47, wherein the cell is a mammalian cell.

Embodiment 49: The method of embodiment 47 or 48, wherein the cell is a mammalian neuronal cell.

Embodiment 50: The method of any one of embodiments 47-49, wherein the cell is a retinal ganglion cell (RGC), a photoreceptor cell, or a retinal pigmented epithelial cell.

Embodiment 51: A method of expressing a first heterologous nucleic acid sequence and a second heterologous nucleic acid sequence, comprising contacting a cell with the nucleic acid of any one of embodiments 26-45 or the expression vector of embodiment 46, and allowing the cell to express the first heterologous nucleic acid sequence and the second heterologous nucleic acid sequence.

Embodiment 52: The method of embodiment 51, wherein the cell is a mammalian cell.

Embodiment 53: The method of embodiment 51 or 52, wherein the cell is a mammalian neuronal cell.

Embodiment 54: The method of any one of embodiments 51-53, wherein the cell is a retinal ganglion cell (RGC), a photoreceptor cell, or a pigmented epithelial cell.

Embodiment 55: A cell comprising the virus of any one of embodiments 1-25, the nucleic acid of any one of embodiments 26-45, or the expression vector of embodiment 46.

Embodiment 56: The cell of embodiment 55, wherein the cell is a mammalian cell.

Embodiment 57: The cell of embodiment 55 or 56, wherein the cell is a mammalian neuronal cell.

Embodiment 58: The cell of any one of embodiments 55-57, wherein the cell is a retinal ganglion cell (RGC), a photoreceptor cell, or a pigmented epithelial cell.

Embodiment 59: A method of treating a disease in a subject in need thereof, the method comprising administering to said subject an effective amount virus of any one of embodiments 1-25, the nucleic acid of any one of embodiments 26-45, or the expression vector of embodiment 46.

Embodiment 60: The method of embodiment 59, wherein the disease is a neurodegenerative disease.

Embodiment 61: The method of embodiment 59 or 60, wherein the disease is glaucoma, age-related macular degeneration (AMD), choroidal neovascularization (CNV), myopia-associated CNV, diabetic retinopathy, macular oedema, and retinal vein occlusion, or retinal pigmentosa.

### EXAMPLES

### Introduction to Exemplary Studies

Gene therapy using adeno-associated virus (AAV) is limited by the virus packaging capacity, approximately 4.7 kilobases (kb), including the inverted terminal repeats (ITRs). The compact sizes of the promoters provided herein, combined with their transcriptional processivity and the ability to drive multiple transcripts, including embodiments thereof, enable delivery of vectors that encode more constructs and allow for expression of longer transcripts than conventional AAV constructs. Using the transgenes and vectors described herein, transcripts encoding therapeutic proteins, CRISPR-associated proteins, guide RNAs, and inhibitory RNAs can be expressed in target cells. In the case of CRISPR systems, this includes all-in-one designs in which both the transcript encoding the Cas protein as well as a transcript encoding an array of gRNAs can be delivered by a single virus.

As described below, 20 candidate bidirectional promoters were tested for expression of GFP one side and RFP on the other side. Of those, only a fraction were shown to be bidirectional and robust, and only one promoter (“Promoter 10”) was active in central nervous system (CNS) neurons (e.g. retinal ganglion cells).

### Example 1: Promoter 10 Drives Robust Bidirectional Transcription in Human Embryonic Kidney (HEK) 293 T Cells

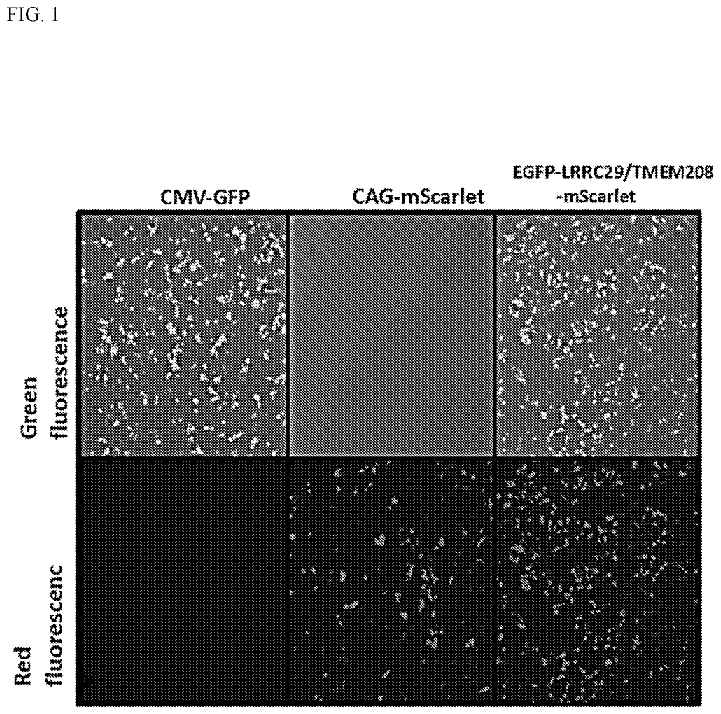

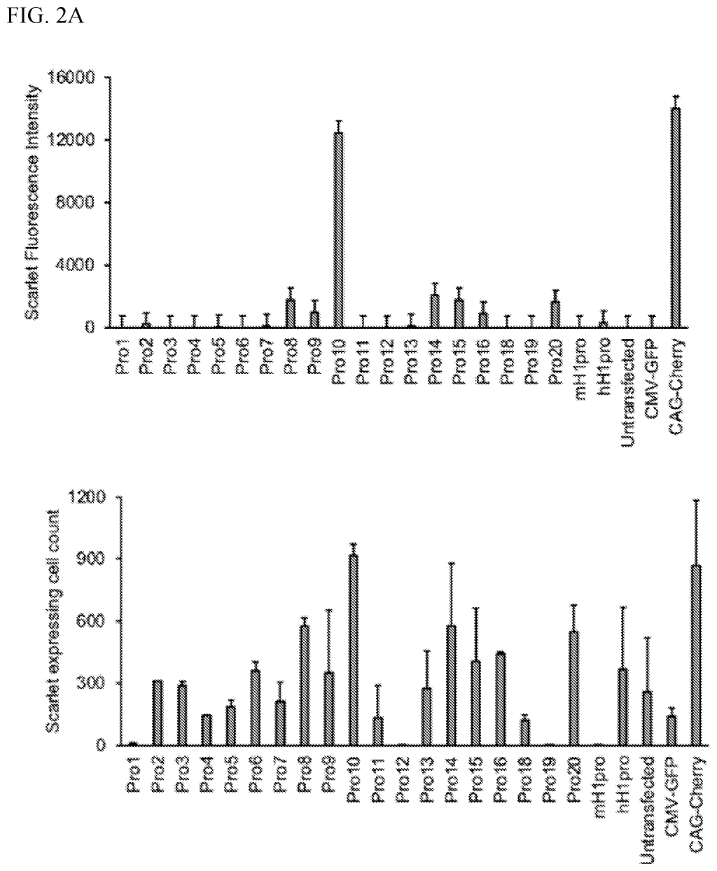

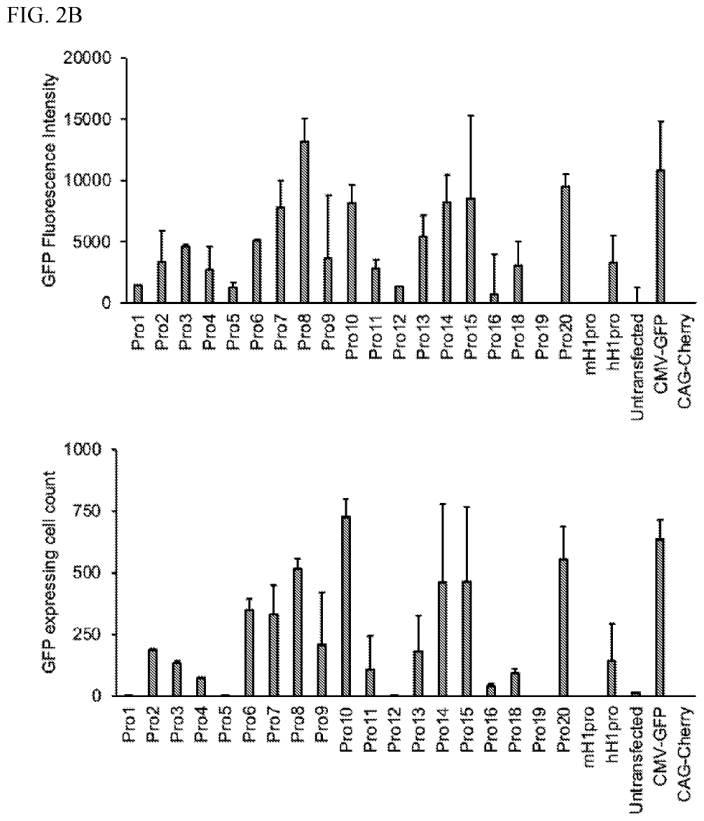

We selected putative bidirectional promoters bioinformatically identified by Trinklein et al., 2004, Genome Res. 14:62-66. We rank-ordered them based on previously reported expression levels (see Trinklein et al.) in four cell lines as unidirectional promoters and selected 20 for further testing. We also focused on candidate promoters less than ˜0.35 kb in length. Each candidate promoter was cloned into a plasmid that would simultaneously and outwardly drive expression of mScarlet and eGFP reporters. A CMV promoter and a CAG promoter were also used to express GFP and mScarlet, respectively for comparison. The plasmids were transfected into human embryonic kidney (HEK) 293T cells and quantified for efficiency of fluorescence expression 24 hours later using the Molecular Devices Image Xpress/MetaXpress system. An image showing expression of GFP and mScarlet using Promoter 10 (EGFP-LRRC29/TMEM208-mScarlet) in comparison to GFP expression obtained with a CMV promoter and mScalret expression obtained with the CAG promoter is provided in FIG. 1. FIG. 2 quantifies expression from the 20 test promoters and comparison CAG and CMV promoters. Unexpectedly, only five of the twenty promoters (Promoters 8, 9, 10, 14, 15) had significant bidirectional expression. Promoter LRRC29/TMEM208 (Promoter 10) (SEQ ID NO:10) had the highest levels of transcription (measured as fluorescent expression) on both sides of the promoter. FIG. 1 shows expression levels comparable to that obtained with CAG or CMV. Given the known problem with promoter interference and elongation from bidirectional promoters (e.g., Curtin et al., Gene Therapy 15:384-390, 2008; Core et al., Science 322:1845-1848, 2008), this suggests that Promoter 10 is uniquely able to avoid interference and simultaneously drive transcription of two coding RNAs.

### Example 2: Promoter 10 Drives Bidirectional Expression in Primary Mouse Retinal Ganglion Cells

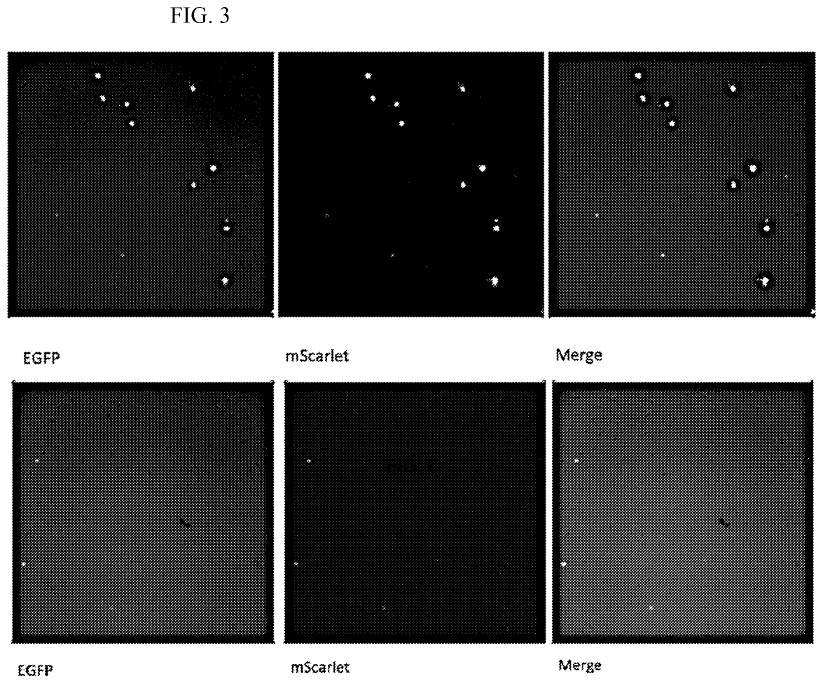

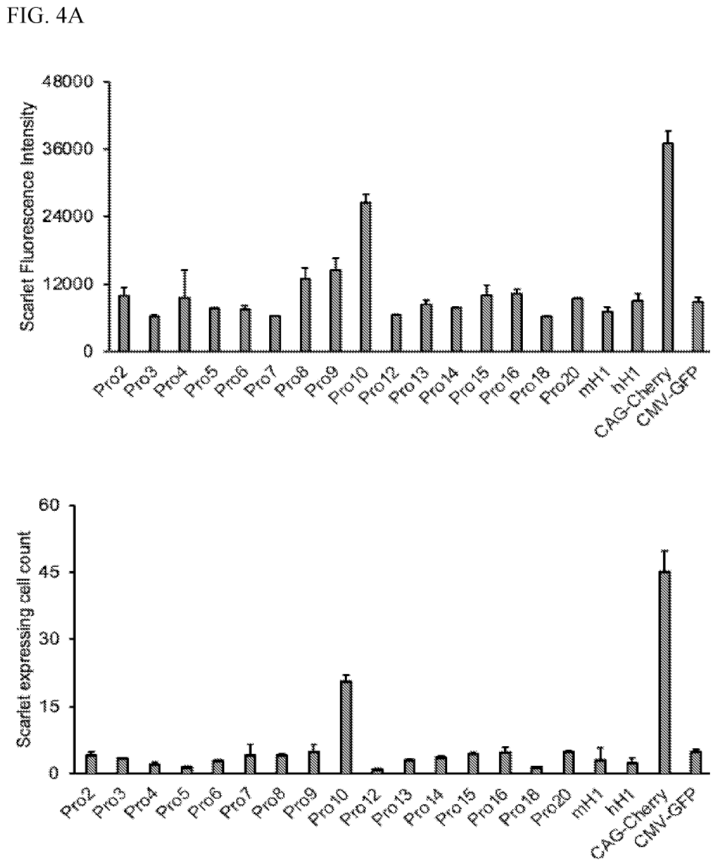

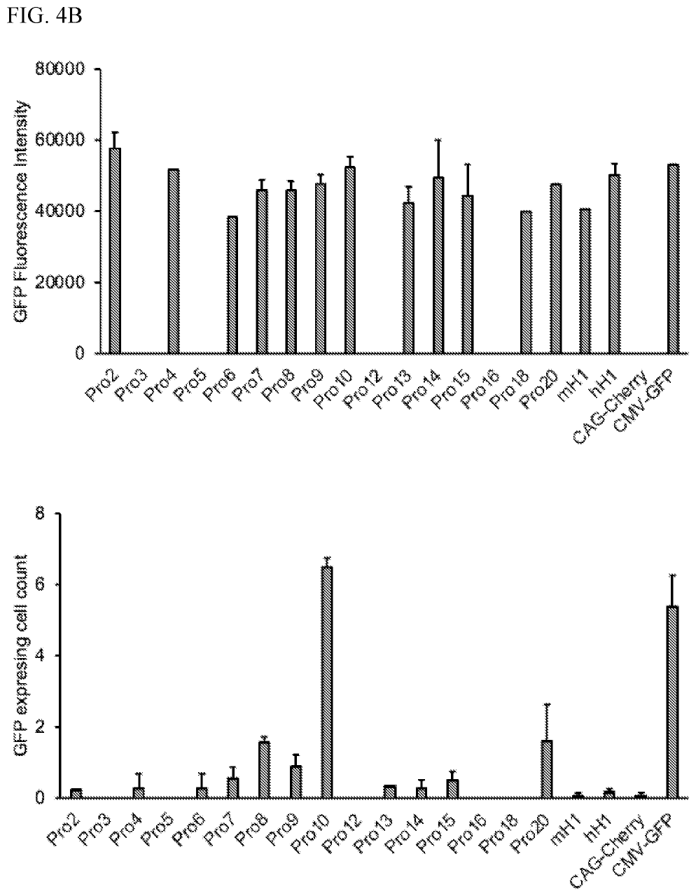

As the transcription factor mileu of a post-mitotic neuron is necessarily different than the dividing cell lines used above, we tested the constructs in CNS neurons. Using a magnetic nanoparticle system (NeuronMag, OzBiosciences), we transiently transfected the plasmids into primary mouse retinal ganglion cells (mRGCs) and imaged green and red fluorescence 24 hours later. Transfection efficacy is low in primary neuronal cells, especially when compared to dividing cell lines such as HEK 293T cells. Therefore the number of plasmid-containing cells is low. Nonetheless, in the sparse transfected cells, Promoter 10 (SEQ ID NO: 10) provided notable bidirectional expression (FIGS. 4A and 4B). Furthermore, green and red fluorescence was observed in the same single cells (FIG. 3). This visualization of green and red in the same cell suggests that the promoter is able to drive transcription off both ends of the promoter in the same cell. Accordingly, two transgenes can be expressed in a single cell.

### Example 3: Mouse Promoter 10 Ortholog

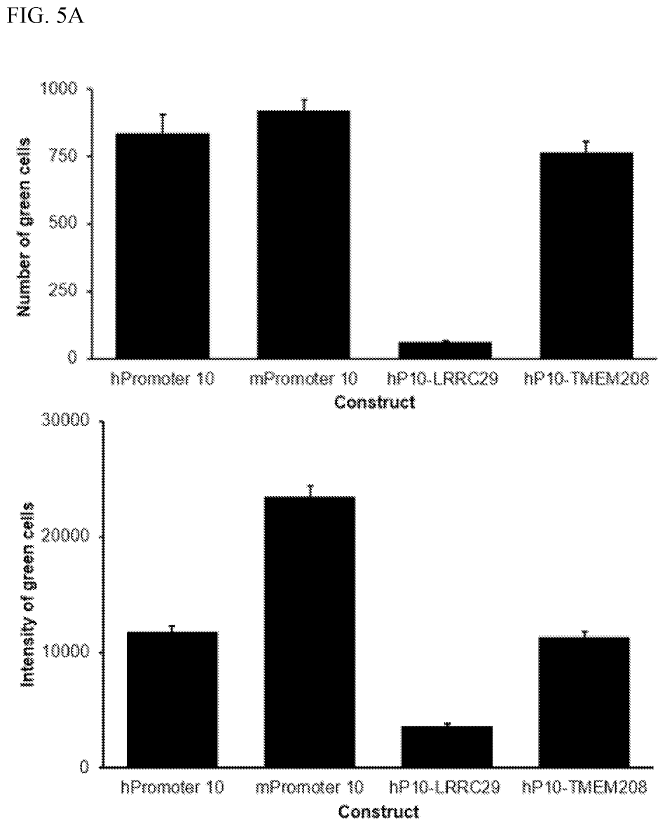

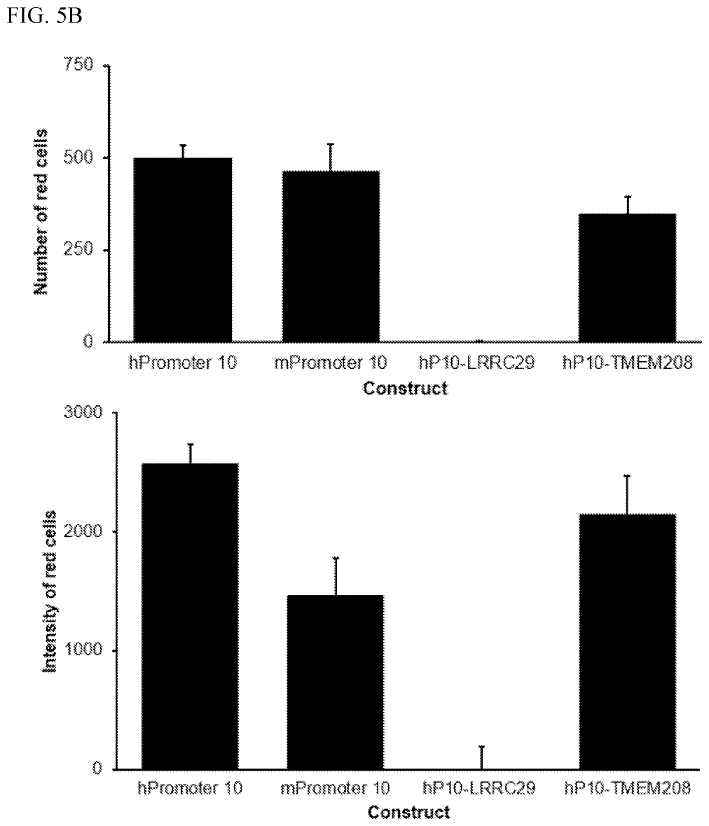

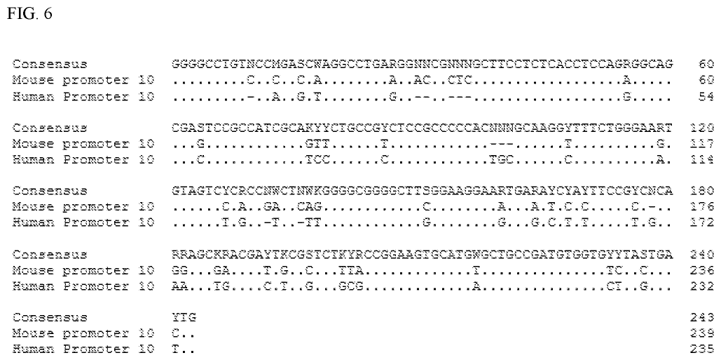

We next tested the mouse ortholog of Promoter 10 (SEQ ID NO:25) in comparison to promoter 10 [SEQ ID NO: 10] and found similar bidirectional transcription in the human cell line HEK 293T (FIGS. 5A and 5B, “mPromoter 10”=mouse promoter 10). An alignment of the human and mouse promoter sequences (FIG. 6) shows a high level of sequence conservation.

### Example 4: Effect of Truncation of Promoter 10

In order to optimize the size of our gene delivery system, we evaluated whether Promoter 10 could be truncated to make the promoter smaller while retaining high levels of bidirectional expression. Promoter activity was evaluated in HEK 293T cells (FIGS. 5A and 5B). We found that truncation of the TMEM208 side (intact LRRC29, plasmid designated as hP10 minus TMEM208 (hP10-TMEM208) of the promoter (SEQ ID NO:21) did not significantly alter gene expression on either side, allowing for viral construct designs with even larger payloads. In contrast, truncation of the LRRC29 side (intact TMEM208, plasmid designated as hP10 minus LRRC29 (hP10-LRRC29) by 67 bp disrupted gene expression in both directions, suggesting the presence of key transcription factor binding sites.

### Example 5: Promoter 10-Driven CRISPR Gene Editing System in Mouse N2A Cells

To select a Cas12a protein and gRNA sequences for use in a Promoter 10 system to perform CRISPR gene editing, we first compared four Cas12a variants. The Cas 12a proteins evaluated were AsCas12a-Ultra (Zhang et al, Nat. Commun. 12:3908, 2021), Lb2Cas12a (Tran et al, Mol Ther. Nucleic Acids 24:40-53, 2021), OpAsCas12a (Gier et al, Nat. Commun. 11:23455, 2020), and enAsCas12a (Kleinstiver et al, Nat Biotechnol. 37 (3): 276-282, 2019). Each Cas12a variant was expressed in a plasmid from one end of promoter 10 and combined with a separate plasmid expressing a single gRNA array targeting the neurodegenerative genes, Dlk and Lzk (also expressed from promoter 10). Conversely, we also generated a series of gRNA variants that targeted Dlk and Lzk to evaluate gRNA designs. Each set of gRNAs was expressed from one side of promoter 10 and combined with a separate plasmid expressing enAsCas12a protein from one end of promoter 10. The preceding experiments were meant to optimize Cas12a and gRNA designs, but did not address which side of the bidirectional promoter might be best for each task (i.e. Cas protein expression and gRNA expression). To address that, human promoter 10 (SEQ ID NO: 10) was cloned into constructs expressing gRNAs from the LRRC29 side or the TMEM208 side. Similarly, Cas12a was cloned with the promoter in both orientations. Plasmids were transfected into N2A cells, which are a mouse neural crest-derived cell line capable of differentiation into neurons.

A Restriction fragment Length Polymorphism (RFLP) assay was used to assess the targeted mutagenesis produced by Cas12a proteins complexed with guide RNAs. For this assay, guide RNAs were selected such that the predicted Cas12a cut site overlapped with restriction enzyme recognition sequences. Cas12a-induced double-stranded breaks (DSB), which are repaired by the endogenous nonhomologous end joining machinery (NHEJ), will generate insertion/deletion mutations (“indels”) that disrupt the restriction site. Accordingly, restriction enzyme cleavage of a locus is inversely proportional to the amount of Cas12a gene editing.

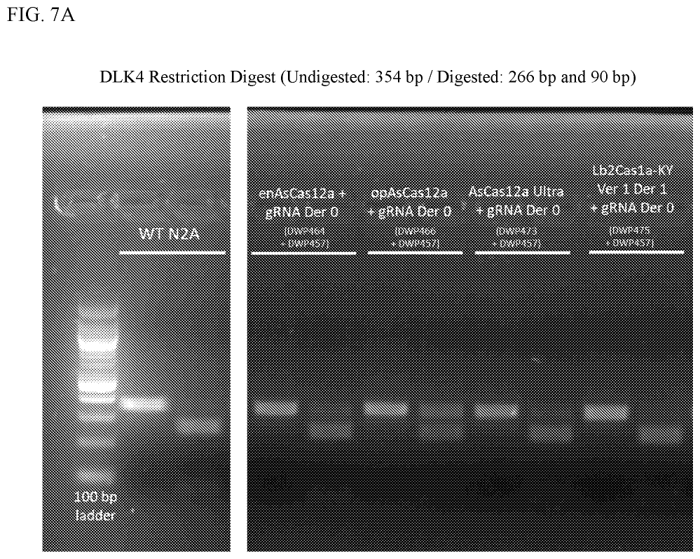

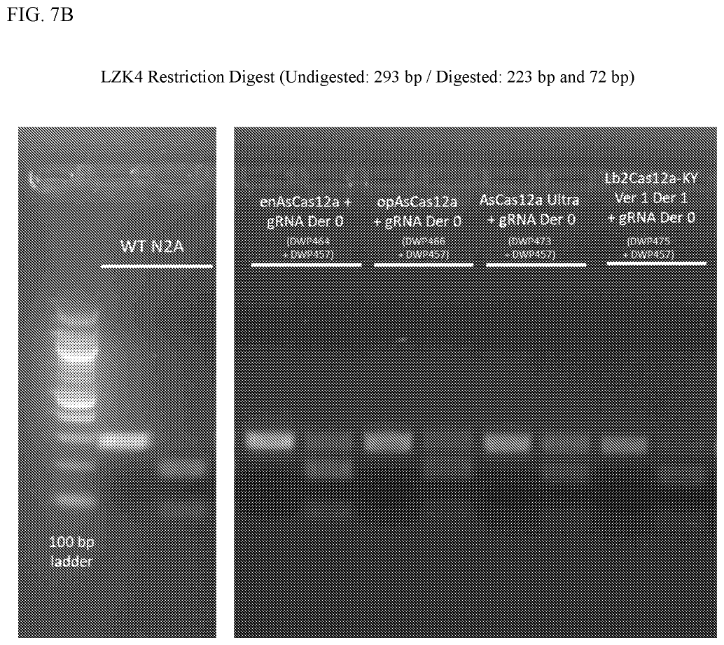

The RFLP assay was performed as follows: PCRs were performed using the N2A extracted genomic DNA (see Methods, below). 200 ng of PCR product was used for restriction endonuclease reactions. Endonuclease AcuI (NEB) was used to evaluate Dlk mutagenesis, and endonuclease AluI (NEB) was used to evaluate Lzk mutagenesis. The reactions were incubated at 37° C. for 1 hour and 80° C. for 20 minutes, following the manufacturer's protocol for the endonucleases. The restriction fragments were separated on 1.5% agarose gel. FIG. 7A shows the results to asses Dlk-targeted gene editing. FIG. 7B shows the results to assess Lzk-targeted gene editing. Each sample has two lanes. The left lane is undigested PCR product. The right lane is DNA incubated with restriction enzyme.

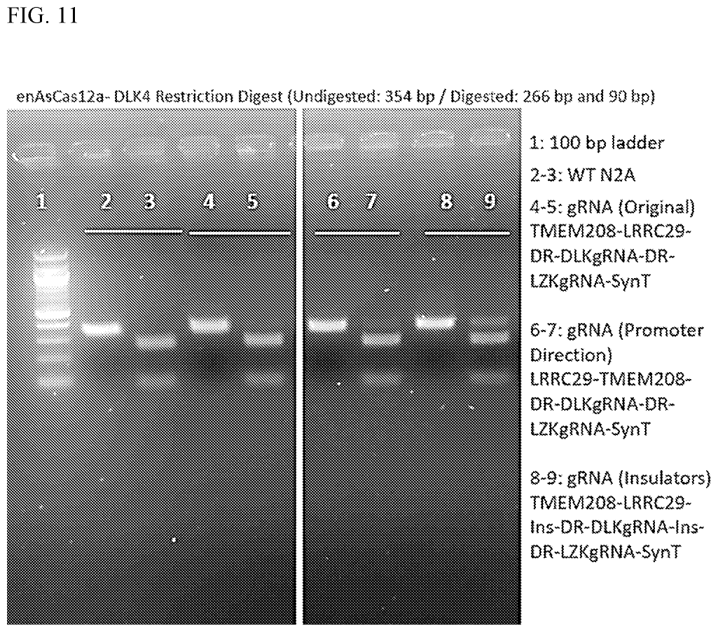

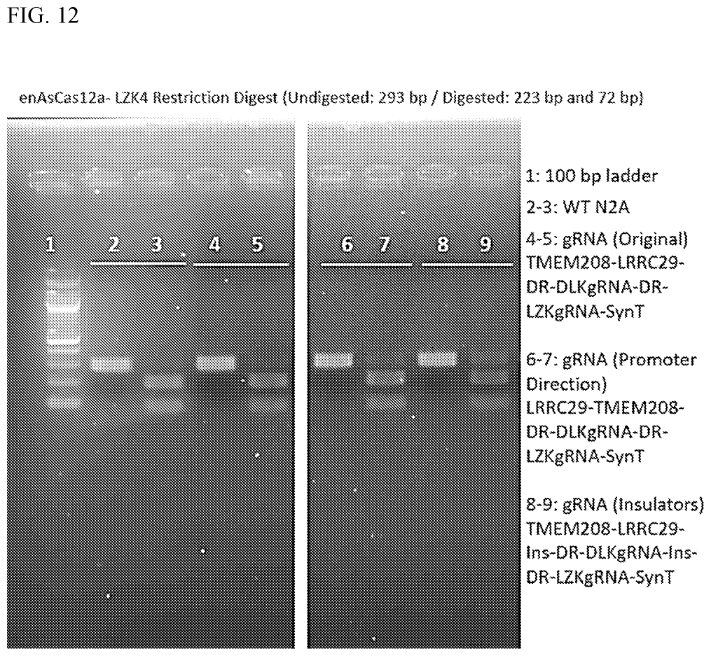

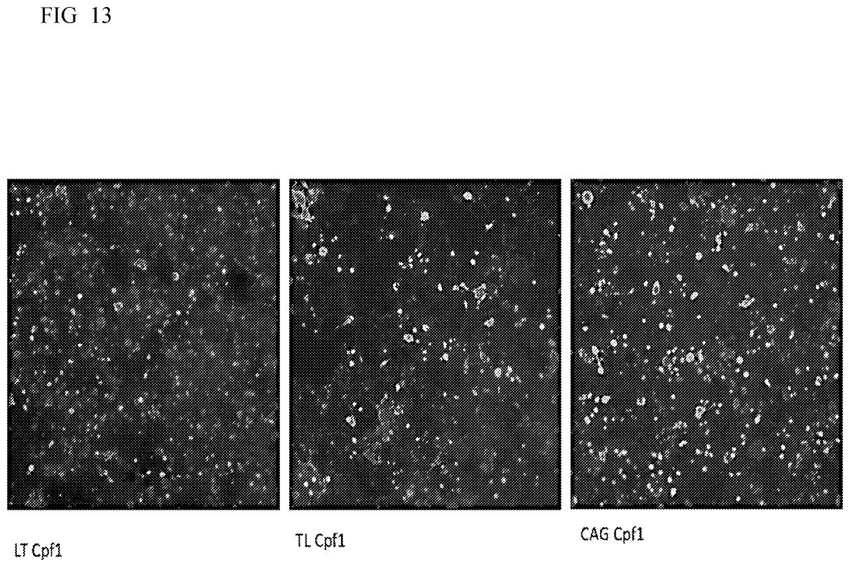

The most pronounced genome editing was seen with opAsCas12a, compared to the other Cas12a variants, at both the Dlk (FIG. 7A) and Lzk (FIG. 7B) loci. Similarly, gRNAs were most effective when expressed off the TMEM208 side (comparing lane 7 vs. 5) at both the Dlk (FIG. 11) and Lzk (FIG. 12) loci. A small spacer sequence, appended to the 3′ end of each gRNA protospacer, also served to improve gene editing (comparing lane 9 vs 5) at both the Dlk (FIG. 11) and Lzk (FIG. 12) loci. Finally, expression of hemagglutinin (HA)-tagged enAsCas12a was measured by anti-HA immunofluorescent staining of 293T cells transfected with promoter 10 driving HA-enAsCas12a off either the TMEM208 or LRRC29 side (FIG. 13). Comparable expression to the control CAG-HA-enAsCas12a construct was seen with expression from either side, with slight preference for the TMEM208 side.

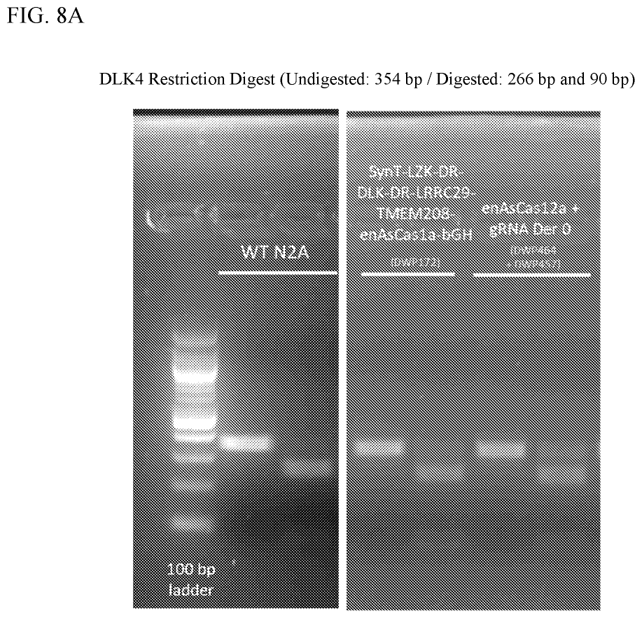

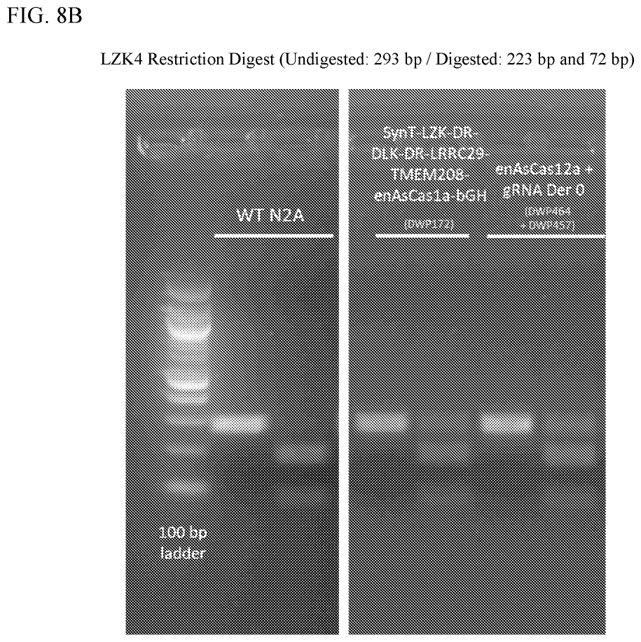

We selected enAsCas12a and created a single plasmid construct to simultaneously drive enAsCas12a expression on the TMEM208 side of Promoter 10 and an array of multiplex gRNAs targeting Dlk and Lzk genes on the LRRC29 side. Gene editing using the single plasmid construct was compared to editing obtained using two separate plasmids (as done above). Gene editing was evaluated with the RFLP assay. FIG. 8A shows the results for Dlk while FIG. 8B shows the results for Lzk. In each case gene editing was observed using the single all-in-one design.

### Example 6: Effect of Introns

In order to further enhance gene expression by promoter 10, we tested whether the inclusion of small introns could help to improve the power of the promoter.

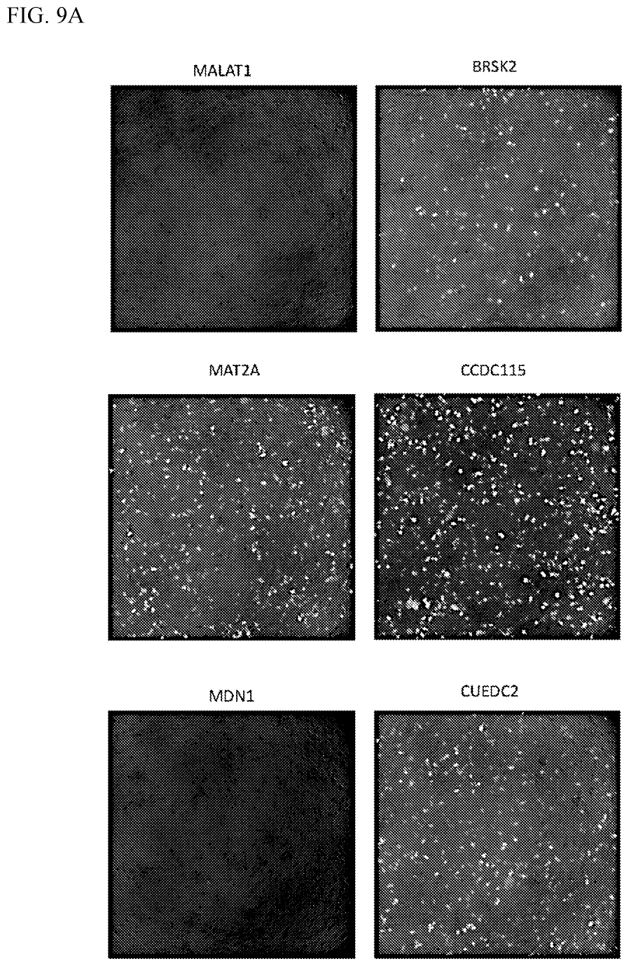

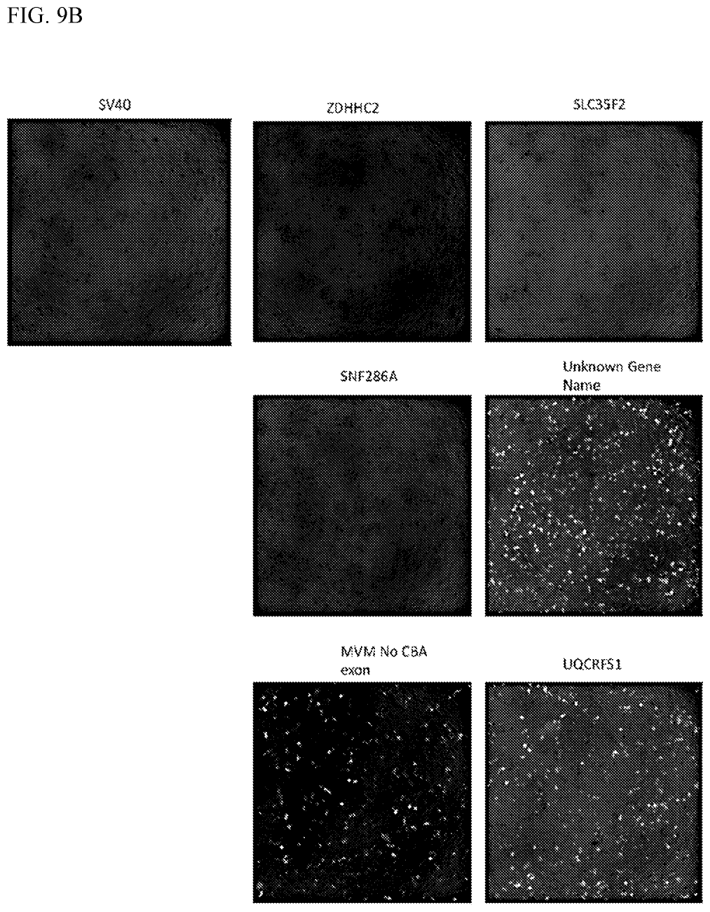

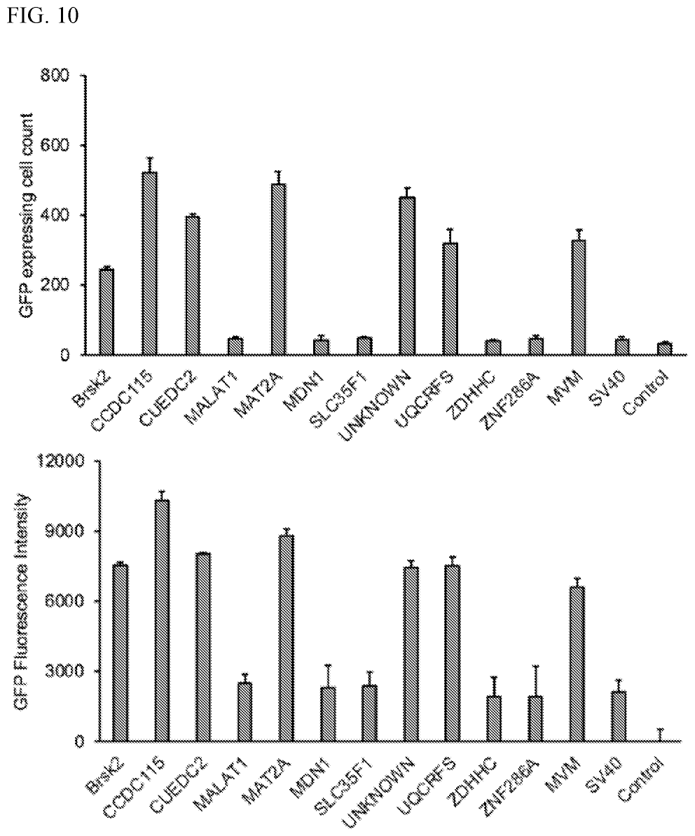

We identified candidate introns using a bioinformatics assessment of small introns across the genome (Abebrese et al., 2017, PLoS One. 2017; 12 (5): e0175393). Candidate introns were subcloned into the following plasmid construct: Promoter 10 (TMEM side)-INTRON-SpyCas9-P2A-EGFP-sNRP terminator. Since the sNRP terminator is not very efficient, there is minimal to no expression of the GFP reporter at baseline. We tested 13 candidate introns, which included, CCDC115, CUEDC2, MAT2A, UQCRFS1, MVM, BRSK2, SV40, ZDHHC2, SLC35F2, SNF286A, an intron from a gene with an unknown name, MVM No CBA, and UQCRFS1 introns, each of which is less than 100 bp. Constructs were transfected into HEK293T cells and assayed for green fluorescence 24 hours later (FIGS. 9-10). The results demonstrated activity from the CCDC115, CUEDC2, MAT2A, UNKNOWN, UQCRFS and MVM introns. The same constructs were then transfected into a CNS neuron and only CCDC115 and CUEDC2 were active.

### Example 7: In Vitro Administration and Expression

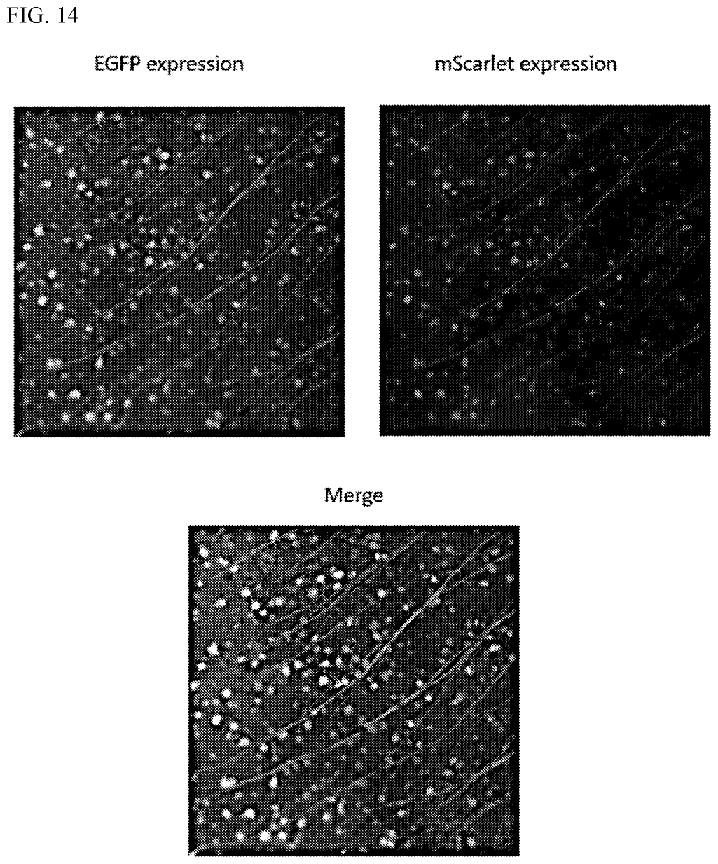

As a first test of whether the promoter worked in the context of an AAV episome, we made a viral construct with Promoter 10 driving bidirectional transcription of mScarlet and EGFP. AAV particles were produced and 10⁹ viral genomes were injected intravitreally into adult C57Bl/6J mice. After two weeks, retinas were flatmounted and examined for red/green fluorescence. The image (FIG. 14) shows robust bidirectional expression of both transgenes. The plane of focus is at the ganglion cell layer/retinal nerve fiber layer (evidenced by the intraretinal axons), indicating expression in retinal ganglion cells.

CRISPR gene editing systems using promoter 10 driving a Cas protein on one side and DLK gRNA on the other +/− an intron enhancement will be evaluated in an optic nerve crush, which is a model in which knockout of DLK is robustly neuroprotective, and RGC survival will be assessed.

### Example 8: Insulator Sequences

The inclusion of small insulator/spacer sequences, for example 5′-AAAT-3′ (SEQ ID NO: 24), was also tested. Inclusion of an insulator spacer sequence improved gene editing when interposed between the gRNAs. (See FIG. 12, lanes 8-9). See, Magnussun et al, eLIFE 2021:10:e66406, 1-20; doi 10.7554/eLife.66406.

### Example 9: Other Alterations

Additional experiments (data not shown) suggest that the terminator sequence following the gRNAs, the length of the gRNAs, the order of the gRNAs, and the nuclear localization signal on the Cas12a enzymes had relatively little effect on gene editing.

### Example 10: Methods

Cell culture: HEK 293 cells were cultured and maintained in DMEM with 10% FBS. Primary mouse RGCs were isolated from P0-P2 mouse pups by dissociating retinas in papain and immunopanning using plates coated with anti-Thy 1.2 antibodies. Cells were seeded into 24 or 96 well plates and transfected with various plasmid constructs using Lipofectamine 3000 or NeuroMag.

Imaging: Cells were imaged at 10× or 20× magnification using an Image Xpress imager and cell number and signal intensity were quantified by MetaXpress image analysis software (Molecular Devices).

Restriction enzyme digest based gene editing assay: CRISPR gene editing target sites that overlapped with a restriction enzyme digest site were selected. A small DNA fragment was generated around the target gene disruption site using PCR. DNA fragments were digested with the appropriate restriction enzyme and lack of digestion of the DNA fragment by the restriction enzyme indicates that the gene target was disrupted by the CRISPR gene editing. Preparation and Extraction of N2A Genomic DNA: Mouse neuroblastoma (N2A) cells were dissociated and plated onto a 6 well plate at 2.5×10⁵ cells per well. The following day, the cells were transfected with 2 μg of Cas12a expression plasmid, 2 μg of gRNA expression plasmid, and 1 μg of EGFP expression plasmid in experiments employing two plasmids for selection of gRNAs and Cas12a variant; or transfected with one expression plasmid containing both Cas12a and an array of gRNAs and eGFP expression plasmid. Thirty six hours after transfection, cells were collected using fluorescent-activated cell sorting (FACS). Collected cells were pelleted by centrifugation at 16,000×g. Genomic DNA was extracted from cells using QuickExtract DNA Extraction solution.

TABLE 2
Sequences of candidate promoters.

| | | | Accession | |
|---|---|---|---|---|
| | | SEQ | numbers of | |
| | | ID | bidirectional | Size |
| Promoter# | Sequence | NO: | genes | (bp) |
| Promoter | ACCACCTCAATGCAGTTGCCGCCGCCTC | 1 | NM_006768/ | 256 |
| 1 | AGCAGCAGCAGCTCCTCGAACAGCCCT | | BC015056 | |
| | CGGAGCCACAACAACCTCCACTTCCGCC | | | |
| | TCCCGGCCGCCGTCGCTGACCTGCGGT | | | |
| | TTACGCATGCGCGCAGGACCGCGCACAG | | | |
| | CGATTGGGCGGCGTGGCAGGGGTCA | | | |
| | CCCACTGCTCTTCCGGACGCAATTTTGA | | | |
| | GGAGGTCGGAGCGGAAGGACGTCGTG | | | |
| | GAGATTGCTTGCGCTGGGGTGCCACACT | | | |
| | TAGGCTGAGCTGCAGGTTTT | | | |
| Promoter | ACCAGCAGCGAGAGAAGGAGCCCGGCA | 2 | AK058000/ | 130 |
| 2 | TGCGGCGACTCAGAGTGACGACACAC | | NM_024093 | |
| | GCGAGTCTCCGCCCGAGTACGTCACTTC | | | |
| | CGCAACGCTTCCTTCGCGGGGCTTTGT | | | |
| | GGGTAGCCGACTGGGGTCTCCT | | | |
| Promoter | AAGAAAATAGGCTTTCTCCGCTCTACCG | 3 | AK025314/ | 239 |
| 3 | CCTCGGGCAGCCACACCTCCACACTTC | | BC005288 | |
| | CGGCGGTGTACCGGCCAAATGCCGCCTG | | | |
| | CCAGCAACTTCCGTCCTCCTAGCTAAA | | | |
| | AGCGGAAAACAGAGGCTCGGAACCGCT | | | |
| | GCGTGGTTCTTGCTCTTCACTCGGCCG | | | |
| | TTTTAAAGGGTGACTCTTTCCTGTCCCGG | | | |
| | CCTGCGTGGTGTGGGCTTGTGGGTCT | | | |
| | TTGAGACCCGAAAATTGAGA | | | |
| Promoter | ATACACATGTGCCAGCCCAGAGCCTCTT | 4 | NM_033662/ | 205 |
| 4 | CCTGTCCGCACCGGTCGGTGACGCCAG | | NM021075 | |
| | GCGCAGACGCCGAGAGATGACGTATACC | | | |
| | CCACGTACCGCGCATGCTCAAGGAC | | | |
| | GAGCCTGCCTTGAGCATGCGTGAAAGGT | | | |
| | GCTGTGACCGCCCTCCGGGTTGTGGC | | | |
| | TGGTGGCCTTCGGTGTTTCGGGTTTGGCA | | | |
| | GTTCACTCACCAAG | | | |
| Promoter | GAGTACTAAGGCGAAGACAGCCATCTTG | 5 | NM_016497/ | 235 |
| 5 | GGCCTTACCCAGGCAGCTGTGGGCGC | | NM_014865 | |
| | ACCGCCCACCTACGCCCCATCCGCAACA | | | |
| | GCCACGAGCCCCGCCCCCCGGGTTCCG | | | |
| | GCAGGAGGGGCCTCTACCACCCTTCCCG | | | |
| | CCTCAGTGCTTCCGGTCCGCCTGCCGG | | | |
| | AAGTTTTGGAGCGCCGGACAAGCTGAGG | | | |
| | TCCGCGACTCGTCGCTAAGATTCCCC | | | |
| | AAAACTGAGATTTCAAG | | | |
| Promoter | GGCTCCTGGAAAAGCGACTCGCGCCTCT | 6 | BC005886/ | 208 |
| 6 | GGGAAGCCGCAGCCCCAGACTCCAGT | | NM_001861 | |
| | CGCGCTTCTCGCCCGGCGCCGCCGGAAA | | | |
| | GCAGCCTCTCCAACGCCTGCCGGAAA | | | |
| | GCAGCCCGGCCCGGCATTTTACGACGTT | | | |
| | CGCAGCGCTACCCTTTTCCGCTCCACG | | | |
| | GTGACCTCCGTGCGGCCGGGTGCGGGCG | | | |
| | GAGTCTTCCTCGATCCC | | | |
| Promoter | GTAGCTATAAAGCGCCAACGAAAAGGA | 7 | NM_021058/ | 306 |
| 7 | AAAACAGCGTGAGCAGGGTATGACAA | | NM_021064 | |
| | GGCGCTTTTATATAGAATCGCTTATGCA | | | |
| | AATAAGGTGAAGAGTTGAAGTCTTGTG | | | |
| | TCTGATTGGTAGTTATTCAGGGTAACGT | | | |
| | CAGAGGTCAGGTCTGCCCAATCAGGA | | | |
| | TTCGCAAATCCAGAAGACGCACTACTAT | | | |
| | TGGTTGAAATTAAACTGCAGCCCTAAC | | | |
| | CAACAACACGTCTTCTTTTTCGCGCCCAA | | | |
| | TAGTGTTTATAAAAAGCGCCGCCTTTC | | | |
| | CCGTTCACTTTGTGGTTGCTCGTAGTGAG | | | |
| | TTGC | | | |
| Promoter | GCTCTACAGCCAGGAAAAGTGAAGCGA | 8 | BC000104/ | 255 |
| 8 | AAACGGCTTCCGTAGACTCCGCCACCA | | BC009228 | |
| | CCGAGTAACAGACCAACTCTGACAGCCC | | | |
| | GAAGATCGCACTTCCGCTTCCGGGGC | | | |
| | ATAGCACCGGAAAGGCTCGTATCGGAGA | | | |
| | ACGGTGTGGCCACGAGAGTCTAGAG | | | |
| | GAAACCAACCCGCAACTCACTGGAGCTG | | | |
| | AAGGGCTGGTAGTCAGGCCGAATCTC | | | |
| | GCGAGAATTGGAACTATTTCAGAGCCAG | | | |
| | AGGAGGATAGTG | | | |
| Promoter | CAGCCTCAGCAAGGATGACTTCCGGCTA | 9 | NM_002109/ | 133 |
| 9 | TCGAGTCGCTGGGTCGCACGCCTAGA | | NM012208 | |
| | GAGCCTCCGCCGAGCACAAAAATTATTT | | | |
| | CCGGCTCCTCCCGGAAGTGCCGAAGC | | | |
| | TGGCTACTAAGGGAACTTGGGAGGA | | | |
| Promoter | GGGGCCTGTCCAGAGCTAGGCCTGAGGG | 10 | NM_012163/ | 234 |
| 10 | CGGCTTCCTCTCACCTCCAGGGGCAG | | NM_014187 | |
| | CGACTCCGCCATCGCATCCCTGCCGCCT | | | |
| | CCGCCCCCACTGCGCAAGGCTTTCTGG | | | |
| | GAAATGTAGTCTCGCCTCTTTGGGGCGG | | | |
| | GGCTTGGGAAGGAAGTGAGACCTATT | | | |
| | TCCGTCGCAAAAGCTGACGACTTCGGTC | | | |
| | TGCGCCGGAAGTGCATGAGCTGCCGA | | | |
| | TGTGGTGCTTAGTGATTG | | | |
| Promoter | CAGACGGAAAGGAAAGAGCTGTACTCCC | 11 | BC030575/ | 295 |
| 11 | ACAATGCCTTTTGGGCTCCTCCCCCAC | | BC012145 | |
| | CTCAGCCACCCTCTTTCCGGTGGAAAGC | | | |
| | TGCGGCCTCCCAGAGCGTCATGGGAC | | | |
| | ATGTAGTTCCGGCATCGCGCCTGGTGGC | | | |
| | GGAGTTCTGCCGAGTGGGGCGCCGC | | | |
| | GGCCGCTATTGTCCCGCCCCCTGCTCCGC | | | |
| | AAGATTCGAGCCTGAGCGGCCTGGG | | | |
| | CGTCTCGAGAGGTGAGAGAGTTGGCGGC | | | |
| | GAGGTCTCGGCGGCTAAGCGAGCGT | | | |
| | CGGCGACTGTCTCTCCGCGAGAGGAG | | | |
| Promoter | GGAAACGCAATGTCAGTTTCCGCGGAAG | 12 | BC016915/ | 250 |
| 12 | AGACCCGCGCCCCGAACCTGTAGCCA | | NM_003611 | |
| | GAACGCCGAAGCAGTTCTCGCGATACCC | | | |
| | TGGGATGTGCTCTCGCGATACGTAGG | | | |
| | CGGAGCTTCCGGCCGGTGCTGGGCAACC | | | |
| | AAGCTCATGCGCCGTAGCTCTTCAGC | | | |
| | TCGGGAAGGCTATATTTAGCAGGTTTCC | | | |
| | GGAAGTTGCCGGACTGGCTGTGAGGCGG | | | |
| | TCCTGCCTCGCTGCCTTCAGTCCCTAG | | | |
| | TGTCT | | | |
| Promoter | GGCTCCATGGTGATGCAGTCGCTGTGCG | 13 | AK023060/ | 213 |
| 13 | ACGCTTCCCGGCGCAGGTTCTGGCGC | | AK055241 | |
| | GTAGTGACGTCACCCGCTCCGCGCCTGC | | | |
| | GCGATGGTAGGAGGGGCGGAGGCTG | | | |
| | AGCGGGCGAGGCCCAGGCGCGCATGCTC | | | |
| | GGGCGGGTAAAATCCGCGCCGGGG | | | |
| | ACAGTCAGGGATGCTCCCAGGCCCTTCG | | | |
| | CTCTTGCTTTCAGCTAGTCATTGTCG | | | |
| Promoter | GGTCCTCTTCTATCCGCAGTCCCGGACCT | 14 | NM_014846/ | 205 |
| 14 | GGAGCGGGTCAGACCCCGACTTCCGC | | AK057002 | |
| | CCCTGACTCCCCAGGCGGTCACATGACC | | | |
| | GAAGCGGCTGGGAGCAGGGAGGGGC | | | |
| | ATCATGTGACCACTCGCCGGCGACGGCT | | | |
| | GATGACGCAAGAGCCCGCTCTCACTTT | | | |
| | TCAGCGGCAGGCGAAGGGGGCTGAGGA | | | |
| | AAGGAGGTGGGTCTAG | | | |
| Promoter | GAAACTCAGATTCTCGGCACCTCCAGCA | 15 | BC005217/ | 235 |
| 15 | GCTGGCTTCGCCAACGGCGTTGAACA | | NM_002940 | |
| | AGGGTCGCAGCTCAATGACGTCATATCT | | | |
| | CCCTACCTACCTCCAGGGTTCCGCCTC | | | |
| | ACGCTCTATGTCGCGCGCGCGCACTACG | | | |
| | TCCTATGGCTTGCGCGTGCGGCGGCT | | | |
| | GGGCACCGCCATTTTGGCCGGTGGCCGT | | | |
| | GAGAACACGCTGTGTGGCTGAAAAGT | | | |
| | GAAGGCAAGAGCTGATTTG | | | |
| Promoter | GCTGTTCTCCGCCATCTTCGCCGCCTGCT | 16 | NM_014821/ | 195 |
| 16 | GGGTTTCCGGCCGGCGCAGCTCAGCC | | BC022361 | |
| | CCCGCCTTGCGTCACGTCCGGCCTGCGA | | | |
| | GTTACCGCCCACTCGCTGCGGCGCTTC | | | |
| | TGGCTCCAGACCGCCCTCCGGATCGGAC | | | |
| | CCTGCGAATGGTTTTGGCTATATC | | | |
| Promoter | GTTCAGGAAGCTCCGCACCTATCCGGCC | 17 | BC027591/ | 219 |
| 17 | GCCACCCTCAGCACAGCCACGGCAGC | | AK055707 | |
| | CGACTCGGCGTTCCTACTGCCCCGGAAG | | | |
| | TGCTCCTTCAGCGCAGAGGCGTGCCG | | | |
| | GTGTGTCCGAGAAAACTTCCGAGTTAAG | | | |
| | CCGCCGCTGAGGCCGGAAGGAGCTA | | | |
| | GACGGCGGTCGGGTAGGTGACGGCTTGG | | | |
| | TTGGGGTCGCTGCGAGGGGACTACT | | | |
| | AGGAG | | | |
| Promoter | GCTGTTCTCCGCCATCTTCGCCGCCTGCT | 18 | BC012464/ | 161 |
| 18 | GGGTTTCCGGCCGGCGCAGCTCAGCC | | NM_005600 | |
| | CCCGCCTTGCGTCACGTCCGGCCTGCGA | | | |
| | GTTACCGCCCACTCGCTGCGGCGCTTC | | | |
| | TGGCTCCAGACCGCCCTCCGGATCGGAC | | | |
| | CCTGCGAATGGTTTTGGCTATATC | | | |
| Promoter | GTTCAGGAAGCTCCGCACCTATCCGGCC | 19 | AK024664/ | 219 |
| 19 | GCCACCCTCAGCACAGCCACGGCAGC | | NM_005927 | |
| | CGACTCGGCGTTCCTACTGCCCCGGAAG | | | |
| | TGCTCCTTCAGCGCAGAGGCGTGCCG | | | |
| | GTGTGTCCGAGAAAACTTCCGAGTTAAG | | | |
| | CCGCCGCTGAGGCCGGAAGGAGCTA | | | |
| | GACGGCGGTCGGGTAGGTGACGGCTTGG | | | |
| | TTGGGGTCGCTGCGAGGGGACTACT | | | |
| | AGGAG | | | |
| Promoter | GAGCAAAGGCCTCACCTCAGCCGCGGAC | 20 | AK056696/ | 194 |
| 20 | AATGGCGGCTGCTTCCCCGGCGTCGAC | | AK000879 | |
| | CGGAGCGCGCCGCGGGGTCTGCTGGGA | | | |
| | GCTGTAGTTCCAGGCGGGCACTTCC | | | |
| | GGCGTCGGGCAGCGGCGCCGCCATGACA | | | |
| | GGTTCCTGGGCCGCGCCGCCTCGCC | | | |
| | CTGCCTGGGCGGGGTTGGGACCTTTCTG | | | |
| | GCACCT | | | |
| Promoter | GGGGCCTGTCCAGAGCTAGGCCTGAGGG | 21 | NM_012163/ | 205 |
| 10 | CGGCTTCCTCTCACCTCCAGGGGCAG | | NM_014187 | |
| (truncated | CGACTCCGCCATCGCATCCCTGCCGCCT | | | |
| TMEM20 | CCGCCCCCACTGCGCAAGGCTTTCTGG | | | |
| 8) | GAAATGTAGTCTCGCCTCTTTGGGGCGG | | | |
| | GGCTTGGGAAGGAAGTGAGACCTATT | | | |
| | TCCGTCGCAAAAGCTGACGACTTCGGTC | | | |
| | TGCGCCGGAAGTGC | | | |

CCDC115 (80 bp) (SEQ ID NO:22)

CAGCCCTGCAGGGAGAGGAAAGCGGTGAGACTCAGTTTAC
GCCCGTGTACCCTCCTGCCCGCCCCAGGCGCCTACCTCCT

CUEDC2 (42 bp) (SEQ ID NO:23):

GGCGGCCCGTCCGGGGCCAGCGGCCGCGACAATAGTCGCGGC

Insulator (SEQ ID NO:24):

5′-AAAT-3′

Mouse Promoter 10 (SEQ ID NO:25):

ggggcctgtc cccgaccaag gcctgaagga ccgctcgctt
cctctcacct ccagaggcag cgagtccgcc atcgcagttc
tgccgtctcc gcccccacgc aaggttttct gggaagtgta
gtcccaccga ctcagggggc ggggcttcgg aaggaaatga
aatccacttc cgcccaggag cgaacgattg cgctctttac
cggaagtgca tgtgctgccg atgtggtgtc tactgactg

Reversed directionality Promoter 10 (SEQ ID NO:26):

caatcactaa gcaccacatc ggcagctcat gcacttccgg
cgcagaccga agtcgtcagc ttttgcgacg gaaataggtc
tcacttcctt cccaagcccc gccccaaaga ggcgagacta
catttcccag aaagccttgc gcagtggggg cggaggcggc
agggatgcga tggcggagtc gctgcccctg gaggtgagag
gaagccgccc tcaggcctag ctctggacag gcccc

Consensus sequence from the alignment of human and mouse promoter 10 sequences (SEQ ID NO:27)

GGGGCCTGTNCCMGASCWAGGCCTGARGGNNCGNNNGCT
TCCTCTCACCTCCAGRGGCAGCGASTCCGCCATCGCAKY
YCTGCCGYCTCCGCCCCCACNNNGCAAGGYTTTCTGGGA
ARTGTAGTCYCRCCNWCTNWKGGGGCGGGGCTTSGGAAG
GAARTGARAYCYAYTTCCGYCNCARRAGCKRACGAYTKC
GSTCTKYRCCGGAAGTGCATGWGCTGCCGATGTGGT
GYYTASTGAYTG

1.-25. (canceled)

26. A polynucleotide comprising:
i) a promoter having bidirectional promoter activity comprising a nucleic acid sequence having at least 85% identity to the sequence of SEQ ID NO:21;
ii) a first heterologous nucleic acid sequence attached to the 3′ end of the promoter; and
iii) a second heterologous nucleic acid sequence attached to the 5′ end of the promoter.

27. The polynucleotide of claim 26, wherein:
the first heterologous nucleic acid sequence encodes a first therapeutic agent or a first detectable agent and the second heterologous nucleic acid sequence is the reverse complement of a sequence encoding a second therapeutic agent or a second detectable agent.

28. The polynucleotide of claim 27, wherein the first therapeutic agent and the second therapeutic agent are independently an RNA-guided DNA endonuclease, an RNA-guided RNA nuclease, a nuclease-deficient RNA-guided DNA endonuclease, a guide RNA (gRNA), a therapeutic protein, or an antibody.

29. The polynucleotide of claim 28, wherein the first therapeutic agent is an RNA-guided DNA endonuclease and the second therapeutic agent is a gRNA.

30.-36. (canceled)

37. The polynucleotide of claim 26, wherein the promoter comprises the sequence of SEQ ID NO:10.

38. The polynucleotide of claim 26, wherein the promoter comprises the sequence of SEQ ID NO:21.

39. The polynucleotide of claim 26, wherein the polynucleotide comprises one or more introns, wherein the intron links the promoter to the first heterologous nucleic acid sequence or the second heterologous nucleic acid sequence.

40. (canceled)

41. The polynucleotide of claim 39, wherein the intron comprises a sequence having at least 95% sequence identity to SEQ ID NO:22 or SEQ ID NO:23.

42.-45. (canceled)

46. An expression vector comprising the polynucleotide of claim 26.

47.-50. (canceled)

51. A method of expressing a first heterologous nucleic acid sequence and a second heterologous nucleic acid sequence in a cell, comprising introducing the polynucleotide of claim 26 into the cell, and allowing the cell to express the first heterologous nucleic acid sequence and the second heterologous nucleic acid sequence.

52.-54. (canceled)

55. A cell comprising the polynucleotide of claim 26.

56. (canceled)

57. The cell of claim 55, wherein the cell is a neuronal cell.

58. The cell of claim 55, wherein the cell is a retinal ganglion cell (RGC), a photoreceptor cell, or a pigmented epithelial cell.

59. A method of treating a neurodegenerative disease in a subject in need thereof, the method comprising administering to said subject an effective amount of the polynucleotide of claim 26.

60. (canceled)

61. The method of claim 59, wherein the disease is glaucoma, age-related macular degeneration (AMD), choroidal neovascularization (CNV), myopia-associated CNV, diabetic retinopathy, macular oedema, and retinal vein occlusion, or retinal pigmentosa.

62. The expression vector of claim 46, wherein the expression vector is a viral vector.

63. The expression vector of claim 62, wherein the viral vector is a herpes simplex virus, an adeno-associated virus (AAV), a lentivirus, an adenovirus, a vaccinia virus, a poxvirus, an alphavirus, an enterovirus, a papillomavirus, or a poliovirus.

64. The expression vector of claim 63, wherein the viral vector is an AAV vector.

65. A virus comprising the polynucleotide of claim 26.

66. The method of claim 51, wherein the polynucleotide is introduced into the cell in vivo.


SEQUENCE LISTING (27 sequences)

SEQ ID NO: 1 - DNA, 265 bp, Homo sapiens
accacctcaatgcagttgccgccgcctcagcagcagcagctcctcgaacagccctcggagccacaacaacctccacttccgcctcccggccgccgtcgctgacctgcggtttacgcatgcgcgcaggaccgcgcacagcgattgggcggcgtggcaggggtcacccactgctcttccggacgcaattttgaggaggtcggagcggaaggacgtcgtggagattgcttgcgctggggtgccacacttaggctgagctgcaggtttt

SEQ ID NO: 2 - DNA, 130 bp, Homo sapiens
accagcagcgagagaaggagcccggcatgcggcgactcagagtgacgacacacgcgagtctccgcccgagtacgtcacttccgcaacgcttccttcgcggggctttgtgggtagccgactggggtctcct

SEQ ID NO: 3 - DNA, 239 bp, Homo sapiens
aagaaaataggctttctccgctctaccgcctcgggcagccacacctccacacttccggcggtgtaccggccaaatgccgcctgccagcaacttccgtcctcctagctaaaagcggaaaacagaggctcggaaccgctgcgtggttcttgctcttcactcggccgttttaaagggtgactctttcctgtcccggcctgcgtggtgtgggcttgtgggtctttgagacccgaaaattgaga

SEQ ID NO: 4 - DNA, 205 bp, Homo sapiens
atacacatgtgccagcccagagcctcttcctgtccgcaccggtcggtgacgccaggcgcagacgccgagagatgacgtataccccacgtaccgcgcatgctcaaggacgagcctgccttgagcatgcgtgaaaggtgctgtgaccgccctccgggttgtggctggtggccttcggtgtttcgggtttggcagttcactcaccaag

SEQ ID NO: 5 - DNA, 235 bp, Homo sapiens
gagtactaaggcgaagacagccatcttgggccttacccaggcagctgtgggcgcaccgcccacctacgccccatccgcaacagccacgagccccgccccccgggttccggcaggaggggcctctaccacccttcccgcctcagtgcttccggtccgcctgccggaagttttggagcgccggacaagctgaggtccgcgactcgtcgctaagattccccaaaactgagatttcaag

SEQ ID NO: 6 - DNA, 208 bp, Homo sapiens
ggctcctggaaaagcgactcgcgcctctgggaagccgcagccccagactccagtcgcgcttctcgcccggcgccgccggaaagcagcctctccaacgcctgccggaaagcagcccggcccggcattttacgacgttcgcagcgctacccttttccgctccacggtgacctccgtgcggccgggtgcgggcggagtcttcctcgatccc

SEQ ID NO: 7 - DNA, 306 bp, Homo sapiens
gtagctataaagcgccaacgaaaaggaaaaacagcgtgagcagggtatgacaaggcgcttttatatagaatcgcttatgcaaataaggtgaagagttgaagtcttgtgtctgattggtagttattcagggtaacgtcagaggtcaggtctgcccaatcaggattcgcaaatccagaagacgcactactattggttgaaattaaactgcagccctaaccaacaacacgtcttctttttcgcgcccaatagtgtttataaaaagcgccgcctttcccgttcactttgtggttgctcgtagtgagttgc

SEQ ID NO: 8 - DNA, 255 bp, Homo sapiens
gctctacagccaggaaaagtgaagcgaaaacggcttccgtagactccgccaccaccgagtaacagaccaactctgacagcccgaagatcgcacttccgcttccggggcatagcaccggaaaggctcgtatcggagaacggtgtggccacgagagtctagaggaaaccaacccgcaactcactggagctgaagggctggtagtcaggccgaatctcgcgagaattggaactatttcagagccagaggaggatagtg

SEQ ID NO: 9 - DNA, 133 bp, Homo sapiens
cagcctcagcaaggatgacttccggctatcgagtcgctgggtcgcacgcctagagagcctccgccgagcacaaaaattatttccggctcctcccggaagtgccgaagctggctactaagggaacttgggagga

SEQ ID NO: 10 - DNA, 235 bp, Homo sapiens
ggggcctgtccagagctaggcctgagggcggcttcctctcacctccaggggcagcgactccgccatcgcatccctgccgcctccgcccccactgcgcaaggctttctgggaaatgtagtctcgcctctttggggcggggcttgggaaggaagtgagacctatttccgtcgcaaaagctgacgacttcggtctgcgccggaagtgcatgagctgccgatgtggtgcttagtgattg

SEQ ID NO: 11 - DNA, 295 bp, Homo sapiens
cagacggaaaggaaagagctgtactcccacaatgccttttgggctcctcccccacctcagccaccctctttccggtggaaagctgcggcctcccagagcgtcatgggacatgtagttccggcatcgcgcctggtggcggagttctgccgagtggggcgccgcggccgctattgtcccgccccctgctccgcaagattcgagcctgagcggcctgggcgtctcgagaggtgagagagttggcggcgaggtctcggcggctaagcgagcgtcggcgactgtctctccgcgagaggag

SEQ ID NO: 12 - DNA, 250 bp, Homo sapiens
ggaaacgcaatgtcagtttccgcggaagagacccgcgccccgaacctgtagccagaacgccgaagcagttctcgcgataccctgggatgtgctctcgcgatacgtaggcggagcttccggccggtgctgggcaaccaagctcatgcgccgtagctcttcagctcgggaaggctatatttagcaggtttccggaagttgccggactggctgtgaggcggtcctgcctcgctgccttcagtccctagtgtct

SEQ ID NO: 13 - DNA, 213 bp, Homo sapiens
ggctccatggtgatgcagtcgctgtgcgacgcttcccggcgcaggttctggcgcgtagtgacgtcacccgctccgcgcctgcgcgatggtaggaggggcggaggctgagcgggcgaggcccaggcgcgcatgctcgggcgggtaaaatccgcgccggggacagtcagggatgctcccaggcccttcgctcttgctttcagctagtcattgtcg

SEQ ID NO: 14 - DNA, 206 bp, Homo sapiens
ggtcctcttctatccgcagtcccggacctggagcgggtcagaccccgacttccgcccctgactccccaggcggtcacatgaccgaagcggctgggagcagggaggggcatcatgtgaccactcgccggcgacggctgatgacgcaagagcccgctctcacttttcagcggcaggcgaagggggctgaggaaaggaggtgggtctag

SEQ ID NO: 15 - DNA, 236 bp, Homo sapiens
gaaactcagattctcggcacctccagcagctggcttcgccaacggcgttgaacaagggtcgcagctcaatgacgtcatatctccctacctacctccagggttccgcctcacgctctatgtcgcgcgcgcgcactacgtcctatggcttgcgcgtgcggcggctgggcaccgccattttggccggtggccgtgagaacacgctgtgtggctgaaaagtgaaggcaagagctgatttg

SEQ ID NO: 16 - DNA, 162 bp, Homo sapiens
gctgttctccgccatcttcgccgcctgctgggtttccggccggcgcagctcagcccccgccttgcgtcacgtccggcctgcgagttaccgcccactcgctgcggcgcttctggctccagaccgccctccggatcggaccctgcgaatggttttggctatatc

SEQ ID NO: 17 - DNA, 219 bp, Homo sapiens
gttcaggaagctccgcacctatccggccgccaccctcagcacagccacggcagccgactcggcgttcctactgccccggaagtgctccttcagcgcagaggcgtgccggtgtgtccgagaaaacttccgagttaagccgccgctgaggccggaaggagctagacggcggtcgggtaggtgacggcttggttggggtcgctgcgaggggactactaggag

SEQ ID NO: 18 - DNA, 162 bp, Homo sapiens; identical to SEQ ID NO: 16

SEQ ID NO: 19 - DNA, 219 bp, Homo sapiens; identical to SEQ ID NO: 17

SEQ ID NO: 20 - DNA, 194 bp, Homo sapiens
gagcaaaggcctcacctcagccgcggacaatggcggctgcttccccggcgtcgaccggagcgcgccgcggggtctgctgggagctgtagttccaggcgggcacttccggcgtcgggcagcggcgccgccatgacaggttcctgggccgcgccgcctcgccctgcctgggcggggttgggacctttctggcacct

SEQ ID NO: 21 - DNA, 205 bp, Homo sapiens
ggggcctgtccagagctaggcctgagggcggcttcctctcacctccaggggcagcgactccgccatcgcatccctgccgcctccgcccccactgcgcaaggctttctgggaaatgtagtctcgcctctttggggcggggcttgggaaggaagtgagacctatttccgtcgcaaaagctgacgacttcggtctgcgccggaagtgc

SEQ ID NO: 22 - DNA, 80 bp, Homo sapiens
cagccctgcagggagaggaaagcggtgagactcagtttacgcccgtgtaccctcctgcccgccccaggcgcctacctcct

SEQ ID NO: 23 - DNA, 42 bp, Homo sapiens
ggcggcccgtccggggccagcggccgcgacaatagtcgcggc

SEQ ID NO: 24 - DNA, 4 bp, Artificial Sequence
  Synthetic construct
aaat

SEQ ID NO: 25 - DNA, 239 bp, Mus musculus
ggggcctgtccccgaccaaggcctgaaggaccgctcgcttcctctcacctccagaggcagcgagtccgccatcgcagttctgccgtctccgcccccacgcaaggttttctgggaagtgtagtcccaccgactcagggggcggggcttcggaaggaaatgaaatccacttccgcccaggagcgaacgattgcgctctttaccggaagtgcatgtgctgccgatgtggtgtctactgactg

SEQ ID NO: 26 - DNA, 235 bp, Homo sapiens
caatcactaagcaccacatcggcagctcatgcacttccggcgcagaccgaagtcgtcagcttttgcgacggaaataggtctcacttccttcccaagccccgccccaaagaggcgagactacatttcccagaaagccttgcgcagtgggggcggaggcggcagggatgcgatggcggagtcgctgcccctggaggtgagaggaagccgccctcaggcctagctctggacaggcccc

SEQ ID NO: 27 - DNA, 243 bp, Artificial Sequence
  Synthetic construct
  misc_feature (10)..(10): n is a, c, g, or t
  misc_feature (30)..(31): n is a, c, g, or t
  misc_feature (34)..(36): n is a, c, g, or t
  misc_feature (99)..(101): n is a, c, g, or t
  misc_feature (132)..(132): n is a, c, g, or t
  misc_feature (136)..(136): n is a, c, g, or t
  misc_feature (178)..(178): n is a, c, g, or t
ggggcctgtnccmgascwaggcctgarggnncgnnngcttcctctcacctccagrggcagcgastccgccatcgcakyyctgccgyctccgcccccacnnngcaaggytttctgggaartgtagtcycrccnwctnwkggggcggggcttsggaaggaartgaraycyayttccgycncarragckracgaytkcgstctkyrccggaagtgcatgwgctgccgatgtggtgyytastgaytg